(19) United States
(12) Patent Application Publication
(10) Pub. No.: US 2024/0294600 A1
(43) Pub. Date: Sep. 5, 2024

(54) ANTI-BCMA CHIMERIC ANTIGEN RECEPTORS

(71) Applicant: Cartesian Therapeutics, Inc., Gaithersburg, MD (US)

(72) Inventors: Yi Zhang, Potomac, MD (US); C. Andrew Stewart, Frederick, MD (US); Metin Kurtoglu, Bethesda, MD (US); Murat V. Kalayoglu, Silver Spring, MD (US); Michael S. Singer, Chestnut Hill, MA (US)

(73) Assignee: Cartesian Therapeutics, Inc., Gaithersburg, MD (US)

(21) Appl. No.: 18/654,279

(22) Filed: May 3, 2024

Related U.S. Application Data
(62) Division of application No. 17/540,947, filed on Dec. 2, 2021, now Pat. No. 11,999,773.
Application No. 17/540,947 is a continuation of application No. 17/156,407, filed on Jan. 22, 2021, now Pat. No. 11,220,535.
Application No. 17/156,407 is a continuation of application No. 16/825,996, filed on Mar. 20, 2020, now Pat. No. 10,934,337.
Application No. 16/825,996 is a continuation of application No. PCT/US2020/022671, filed on Mar. 13, 2020.
(60) Provisional application No. 62/819,068, filed on Mar. 15, 2019.

(51) Int. Cl. C07K 14/705; A61K 39/00; A61K 39/395; C07K 16/28; C07K 16/30

(52) U.S. Cl. CPC C07K 14/70517; A61K 39/3955; A61K 39/39558; C07K 16/2878; C07K 16/2896; C07K 16/30; A61K 2039/505; C07K 2317/24; C07K 2317/31; C07K 2317/53; C07K 2317/565; C07K 2317/622; C07K 2317/73; C07K 2317/76; C07K 2319/03

(57) ABSTRACT

Provided herein are chimeric antigen receptors (CARs), such as those specific for BCMA, that have improved properties, including increased CAR T cell binding to BCMA and improved CAR T cell killing of BCMA-expressing cancer cells. Use of the CARs in immune cells (e.g., T cells), compositions (e.g., CARs and nucleic acid constructs encoding the same), and methods are also contemplated.

### BACKGROUND

B cell maturation antigen (BCMA) is a tumor necrosis family receptor (TNFR) member expressed on cells of the B cell lineage. BCMA expression is the highest on terminally differentiated B cells. BCMA is involved in mediating the survival of plasma cells for maintaining long-term humoral immunity. The expression of BCMA has been recently linked to a number of cancers, autoimmune disorders, allergic disorders, and infectious diseases. Cancers with increased expression of BCMA include some hematological cancers, such as multiple myeloma, Hodgkin's and non-Hodgkin's lymphoma, various leukemias, and glioblastoma. Autoimmune diseases linked to BCMA include, without limitation, myasthenia gravis, systemic lupus erythematosus (SLE), rheumatoid arthritis, blistering skin diseases, e.g., pemphigus, psoriasis, inflammatory bowel disease, celiac sprue, pernicious anemia, idiopathic thrombocytopenia purpura, scleroderma, Graves' disease, Sjögren syndrome, Goodpasture syndrome, and type 1 diabetes. Many autoantibody-mediated autoimmune diseases require chronic treatment with systemic steroids, or immunosuppressants, which involve significant toxicity. Allergic disorders linked to BCMA include anaphylaxis, asthma, food allergy, stinging insect allergy, drug allergy, allergic rhinitis, urticaria, angioedema, eczema, atopic dermatitis, contact dermatitis, and eosinophilic esophagitis. Many allergic diseases require chronic treatment with systemic or local steroids, immunomodulatory therapy, or immunotherapy. Patients affected by environmental, food, drug, and insect allergies often must modify their lifestyles to avoid the offending allergens.

A promising new approach to treating BCMA-related conditions is adoptive transfer of T cells genetically modified to recognize malignancy-associated antigens. See, e.g., U.S. Pat. No. 9,765,342 to Kochenderfer. T cells can be genetically modified by introduction of a nucleic acid construct to express chimeric antigen receptors (CARs), which are fusion proteins comprising an extracellular antigen recognition moiety and an intracellular signaling domain. See, e.g., Eshhar et al., Proc. Natl. Acad. Sci. USA, 1993; 90:720-724, and Sadelain et al., Curr. Opin. Immunol, 2009; 21:215-223. CAR T cells, such as those modified to recognize BCMA, have shown benefit in treating conditions such as multiple myeloma. See, e.g., Ali et al., Blood, 2016; 128:1688-1700; Brudno et al., J. Clin. Oncol., 2018; 36:2267-2280.

CAR proteins and CAR T cells directed against BCMA have previously been described. See, e.g., U.S. Pat. No. 9,765,342 to Kochenderfer; U.S. Pat. No. 10,174,095 to Brogdon et al., and U.S. Pat. App. Pub. No. 2017/0226216 A1 of Morgan et al., the disclosures of which are incorporated herein by reference.

Previously disclosed forms of anti-BCMA CAR T cell therapy suffer from several drawbacks, including manufacturing difficulties, suboptimal expression, suboptimal target engagement and tumor killing, inflammatory side effects, and immunogenicity.

Therefore, there is a need for improved CAR compositions and methods for the treatment of BCMA-related conditions.

### SUMMARY OF THE INVENTION

Some aspects of the disclosure are based at least in part on the surprising discovery that novel constructs expressing modified chimeric antigen receptors demonstrate improved properties, including increased binding to BCMA, and killing of multiple myeloma cells in vitro and in vivo. Exemplary improvements include modification of CDR sequences that cause CAR T cells to bind more effectively to BCMA and are more efficient at killing cancer cells; 5′ and 3′ untranslated sequences (UTRs) that improve CAR expression; cytoplasmic domains that improve CAR expression; and other improvements, such as improved polyadenine (polyA) tails that also improve expression.

In some aspects, the disclosure provides chimeric antigen receptor (CAR) specific for BCMA. In some embodiments, the CAR comprises at least one novel CDR sequence from any one of SEQ ID NOs: 2, 3, 5, 6, 8, 9, 11, 13, 14, 15, 17, or 18. In a particular embodiment, the CAR comprises the CDR sequences (i.e. CDRH1, CDRH2, CDRH3, CDRL1, CDRL2, and CDRL3) of SEQ ID NOs: 2, 6, 9, 11, 14, and 17.

In some embodiments, the CAR further comprises a transmembrane domain. In some embodiments, the transmembrane domain is a CD8 transmembrane domain. However, other transmembrane domains may be used in accordance with the invention. In some embodiments, the CAR further comprises a costimulatory domain and/or a signaling domain. In some embodiments, the costimulatory domain is a 4-1BB costimulatory domain, a CD28 costimulatory domain and/or an OX40 costimulatory domain. In some embodiments, the CAR comprises a signal transduction domain. In some embodiments, the signal transduction domain is a CD3zeta signal transduction domain. In some embodiments, the CD3 zeta signal transduction domain is comprised in the CDR with one or more costimulatory domains (e.g., 41BB, CD28, and/or OX40 costimulatory domains).

Other aspects of the disclosure relate to a nucleic acid comprising a sequence that encodes a CAR as described in any of the above embodiments or as otherwise described herein. In some embodiments, any of the nucleic acid sequences provided herein include one or more features that are useful for improving expression of the CAR. For example, the nucleic acid may include, a 5′ untranslated region (5′ UTR), a 3′ untranslated region (3′ UTR), a polyadenine tail (polyA), a 7-methylguanosine cap (m⁷G), and/or an open reading frame. The nucleic acid may further include an internal ribosome entry site (IRES).

Yet other aspects relate to an mRNA construct suitable for introduction into a cell, e.g., by electroporation as described in any one of the above embodiments or as otherwise described herein. Yet other aspects of the disclosure relate to a vector comprising a nucleic acid as described in any one of the above embodiments or as otherwise described herein. In some embodiments, the vector is lentiviral vector.

Other aspects of the disclosure relate to a cell comprising a CAR and/or a CAR-encoding nucleic acid as described in any one of the above embodiments or as otherwise described herein. In some embodiments, the cell is a stem cell, NK cell, or T cell. In some embodiments, the cell is a T cell.

Other aspects of the disclosure relate to a composition comprising a plurality of a cell (e.g., a T cell) comprising a CAR as described in any one of the above embodiments or as otherwise described herein. In some embodiments, the composition further comprises a pharmaceutically acceptable carrier.

Yet other aspects of the disclosure relate to a method of generating a plurality of CAR modified cells, the method comprising introducing a CAR-encoding nucleic acid described in any one of the above embodiments or as otherwise described herein into a plurality of immune cells by means of electroporation, physical disruption of a cell, e.g., cell squeezing, or by use of a nanoparticle that comprises the nucleic acid. In some embodiments, the immune cells are T cells. In some embodiments, an mRNA construct comprises the CAR-encoding nucleic acid.

Yet other aspects of the disclosure relate to a method of generating a plurality of CAR modified cells, the method comprising introducing a lentiviral vector comprising a nucleic acid as described in any one of the above embodiments or as otherwise described herein into a plurality of immune cells. In some embodiments, one or more lentiviral vectors is provided comprising one or more nucleic acids in any one of the above embodiments or as otherwise described herein. In some embodiments, the immune cells are T cells.

Other aspects of the disclosure relate to a method of treating a subject having cancer or at risk of having cancer, the method comprising administering a T cell comprising a CAR as described in any one of the above embodiments or as otherwise described herein, a composition as described in any one of the above embodiments or as otherwise described herein, or a plurality of cells produced by a method as described in any one of the above embodiments or as otherwise described herein, into a subject having cancer or at risk of having cancer. In some embodiments, the cancer is multiple myeloma.

Also provided are methods of treating a subject having an autoimmune disease or an allergic disorder. Also provided are uses of one or more T cells comprising a CAR, as disclosed herein, for the treatment of cancer, an autoimmune disease, or an allergic disorder.

### BRIEF DESCRIPTION OF THE DRAWINGS

FIG. 1 shows an assessment of cell viability, CAR expression, and BCMA binding by CAR T cells generated with the CAR of SEQ ID: 31 or a control (no mRNA) at 24 hours post transfection.

FIG. 2 shows measurements of multiple myeloma MM.1S cell tumors in vivo following treatment with humanized anti-BCMA CAR T cells generated with a CAR of SEQ ID: 20, 21 or 25. The CAR T cells or control (CAR-negative) cells were administered as a single dose on day 6. Tumor burden is indicated as mean bioluminescence intensity (Flux, photons/second) from fluorescent tumor cells.

FIG. 3 shows measurements of CAR expression, anti-BCMA cytotoxicity, and interferon-γ secretion by T cells that were modified by the Sleeping Beauty transposon system to express a CAR of SEQ ID: 31.

### DEFINITIONS

As used herein and in the claims, the singular forms “a,” “an,” and “the” include the singular and the plural reference unless the context clearly indicates otherwise. Thus, for example, a reference to “an agent” includes a single agent and a plurality of such agents.

The term “activation”, as used herein, refers to the state of a T cell, NK cell, stem cell or other cell that has been sufficiently stimulated to induce detectable cellular proliferation. Activation can also be associated with induced cytokine production, and detectable effector functions. The term “activated T cells” refers to, among other things, T cells that are undergoing cell division.

The terms “allergy” and “allergic”, as used herein, refer to a medical condition involving an abnormal hypersensitivity reaction to an ordinarily harmless substance, i.e., an allergen. Exemplary allergic conditions include anaphylaxis, asthma, food allergy, stinging insect allergy, drug allergy, allergic rhinitis, urticaria, angioedema, eczema, atopic dermatitis, contact dermatitis, and eosinophilic esophagitis.

The term “antibody”, as used herein, broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Non-limiting embodiments thereof are discussed below.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass.

The term “antigen-binding portion” of an antibody (or simply “antibody portion”), as used herein, refers to one or more fragments of an antibody that retain the ability to specifically bind to an antigen (e.g., BCMA). It has been shown that the antigen-binding function of an antibody can be performed by fragments of a full-length antibody. Such antibody embodiments may also be bispecific, dual specific, or multi-specific formats; specifically binding to two or more different antigens. Multispecific, dual specific, and bispecific antibody constructs are well known in the art and described and characterized in Kontermann (ed.), Bispecific Antibodies, Springer, NY (2011), and Spiess et al., Mol. Immunol. 67(2):96-106 (2015).

Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag, New York, 790 pp. (ISBN 3-540-41354-5).

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations, kappa and lambda light chains refer to the two major antibody light chain isotypes.

The term “synthetic antibody” as used herein, refers an antibody which is generated using recombinant DNA technology, such as, for example, an antibody expressed by a viral vector. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

In some embodiments, the term “antigen” or “Ag” as used herein is defined as a molecule that provokes an immune response. This immune response may involve either antibody production, or the activation of specific immunologically competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “tumor antigen” as used herein refers to an antigen associated with a cancer cell, such as a multiple myeloma cell. Examples of tumor antigens include but are not limited to BCMA.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “autoimmune” refers to a disease or illness wherein an individual's immune system, or a component thereof, attacks that individual's normal body tissue(s). An autoimmune disease can be mediated by an autoantibody, i.e., an antibody produced by an individual that recognizes an antigen of that individual's own tissue(s). Exemplary autoimmune diseases include myasthenia gravis, systemic lupus erythematosus (SLE), rheumatoid arthritis, blistering skin diseases, e.g., pemphigus, psoriasis, inflammatory bowel disease, celiac sprue, pernicious anemia, idiopathic thrombocytopenia purpura, scleroderma, Graves' disease, Sjögren syndrome, Goodpasture syndrome, and type 1 diabetes.

The term “autologous” as used herein, is meant to refer to any material derived from the same individual to which it is later to be re-introduced into the individual.

The term “allogeneic” as used herein refers to a graft derived from a different animal of the same species. “Xenogeneic” refers to a graft derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some embodiments, the cancer is a cancer that expresses BCMA. Exemplary cancers that express BCMA include multiple myeloma, Hodgkin lymphoma, non-Hodgkin lymphoma, chronic lymphocytic leukemia (CLL), and glioblastoma. In some embodiments, cancer refers to multiple myeloma. Multiple myeloma is a cancer of plasma cells. Multiple myeloma can be diagnosed with blood tests (serum protein electrophoresis, serum free kappa/lambda light chain assay), bone marrow examination, urine protein electrophoresis, and/or X-rays of commonly involved bones. In some embodiments, cancer refers to Hodgkin's lymphoma (HL). HL is a cancer of B cells.

An “effective amount” refers to the amount of a therapy which is sufficient to reduce or ameliorate the severity and/or duration of a disorder or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent).

As used herein, the term “exogenous” refers to any material introduced from or produced outside an organism, cell, tissue or system.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide. An expression vector can provide for a self-amplifying RNA, e.g., by an RNA viral vector. See, e.g., U.S. Ser. No. 12/831,252.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function but may serve as an antigen receptor. IgE is the immunoglobulin that mediates immediate hypersensitivity by causing release of mediators from mast cells and basophils upon exposure to allergen.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, a “nucleotide sequence or nucleic acid encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

A “lentivirus” as used herein refers to a genus of the Retroviridae family. Lenti viruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lenti viruses. Vectors derived from lenti viruses offer the means to achieve significant levels of gene transfer in vivo, ex vivo or in vitro.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

“Codon-optimized” means that codons relating to a specific amino acid are optimized for translational efficiency of a gene of interest. Codon optimization typically involves evaluating the gene or sequence of interest and substituting the codon with a more prevalent or common codon used for the same amino acid in a specific cell or species. Programs used by those in the art to evaluate codon optimization include those provided by Integrated DNA Technologies, EnCor Biotechnology, Inc., JCat, OptimumGene™ (GenScript USA, Inc., Pisataway, NJ 08854), etc. The sequences encoding the CAR embodiments described herein may be codon-optimized, which can increase their translational efficiency.

The term “linker,” as used herein, refers to a bond (e.g., covalent bond), chemical group, or a molecule linking two molecules or moieties, e.g., two domains of a fusion protein, such as, for example, a nuclease-inactive Cas9 domain and a nucleic acid-editing domain (e.g., an adenosine deaminase). In some embodiments, a linker joins a gRNA binding domain of an RNA-programmable nuclease, including a Cas9 nuclease domain, and the catalytic domain of a nucleic-acid editing protein. In some embodiments, a linker joins a dCas9 and a nucleic-acid editing protein. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” or “overexpression” is intended to indicate an abnormal level of expression (e.g., of the tumor antigen) in a cell from a disease area (e.g., a solid tumor within a specific tissue or organ of the patient) relative to the level of expression in a normal cell from that tissue or organ. Patients having solid tumors or a hematological malignancy characterized by overexpression of the tumor antigen can be determined by standard assays known in the art.

“Parenteral” administration of an immunogenic composition includes, e.g., subcutaneous (s.c), intravenous (i.v.), intramuscular (i.m.), or intrasternal injection, or infusion techniques.

The terms “patient,” “subject,” “individual,” and the like are used interchangeably herein, and refer to any animal, or cells thereof whether in vitro or in situ, amenable to the methods described herein. In some embodiments, the patient, subject or individual is a human.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner. A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

By the term “specifically binds” or “specific for”, as used herein with respect to an antibody (such as a scFv), is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “subject” is intended to include living organisms in which an immune response can be elicited (e.g., mammals). Examples of subjects include humans, dogs, cats, mice, rats, and transgenic species thereof. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development. In some embodiments, the subject has cancer (e.g., multiple myeloma). In other embodiments, the subject is a healthy volunteer.

The term “therapeutic” as used herein means a treatment and/or prophylaxis. A therapeutic effect is obtained by suppression, remission, or eradication of a disease state.

The term “therapeutically effective amount” as used herein, refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

### DETAILED DESCRIPTION OF CERTAIN EMBODIMENTS

Chimeric antigen receptor (CAR) technology is an anti-cancer immune therapy approach aimed at generating effector T cells to target specific tumor antigens. CARs are recombinant receptors that provide both antigen-binding and T cell-activating functions. CARs are known in the art and have been described previously, for example in Sadelain M., et al . . . “The basic principles of chimeric antigen receptor (CAR) design” Cancer Discov. 2013 April; 3(4): 388-398; the entire contents of which are hereby incorporated by reference. In general, CAR T cells (also referred to herein as CART cells or CARTs) are engineered T cells based on a single chain Fv (scFv) antibody moiety. In some embodiments, the chimeric antigen receptor (CAR) portion comprises a receptor complex that combines an antigen binding domain (e.g., scFv) and a signal transduction domain of T cells (such as including the CD3ζ chain).

As provided herein, anti-B-Cell Maturation Antigen (BCMA) CARs, and their corresponding nucleic acid expression constructs, were engineered to confer improved properties including improved expression, improved CAR T cell binding to BCMA, improved half-life, improved killing of BCMA-expressing cells (e.g., myeloma cells), and lower immunogenicity in humans. To generate nucleic acid constructs that express improved anti-BCMA CARs, mutations were made in one or more of the complementarity determining regions (CDRs), mutations were introduced into the open reading frame (ORF), mutations were introduced into the framework region, alternative 5′ and 3′ untranslated regions (UTRs) were used, alternative costimulatory domains were used, and alternative poly-adenine tails were designed. Such improved anti-BCMA CARs and their expression constructs demonstrate improved properties as compared to previously described anti-BCMA CARs, such as the CAR used in the clinical trial of Ali et al. (2016), i.e., of SEQ ID: 19.

As described herein, CARs were engineered based on scFvs that bind to BCMA, a multiple myeloma (MM) antigen. An exemplary CAR can consist of a CD8 signal peptide, an anti-BCMA scFv (e.g. of SEQ ID NO: 60), a CD8 hinge and transmembrane domain, a 4-1BB costimulatory domain, and CD3ζ signal transduction domain. To generate CAR T cells, mRNA constructs expressing CARs were transfected into CD8+ lymphocytes obtained by apheresis from healthy human donors. CAR T cells expressing one of the CAR proteins tested (e.g., CAR T cells expressing the CAR of SEQ ID NO: 21), demonstrated increased binding to BCMA, increased killing of BCMA-expressing myeloma cells in vitro, and significant inhibition of growth of human myeloma tumors in an animal model. Accordingly, aspects of the invention relate to anti-BCMA CARs and methods of use thereof. In some embodiments, the CAR is expressed by a T cell and is useful for treatment of cancer, e.g., for treatment of multiple myeloma.

In some embodiments, the CAR is specific for BCMA. B cell maturation antigen (BCMA, also known as BCM, CD269, TNFRSF17, or TNFRSF13A) is a member of the TNFR superfamily expressed on B cells. An exemplary, non-limiting human BCMA sequence is provided below.
>gi|23238192|ref|NP_001183.2| tumor necrosis factor receptor superfamily member 17, BCMA [Homo sapiens] MLQMAGQCSQNEYFDSLLHACIPCQLRCSSNTPPLICQRYCNASVINSVKGTNAILWTCLGLSLIISLAV FVLMFLLRKINSEPLKDEFKNTGSGLLGMANIDLEKSRIGDEIILPRGLEYTVEECTCEDCIKSKPKVDS DHCFPLPAMEEGATILVTTKINDYCKSLPAALSATEIEKSISAR (SEQ ID NO: 121)

In some embodiments, any of the CARs provided herein comprise a single chain Fv (scFv) that specifically binds to BCMA, which may be on a single polypeptide (e.g., connected by a linker) or on two polypeptides (e.g., one on a first CAR polypeptide and one a second CAR polypeptide, which may form a dimer once introduced into a cell).

### Compositions

### CARs

In some aspects, the present invention provides a chimeric antigen receptor (CAR) specific for BCMA (e.g., that specifically binds to BCMA). In some embodiments the CAR comprises an antigen-binding domain specific for BCMA, a transmembrane domain and a cytoplasmic domain. In some embodiments, the anti-BCMA binding domain is a humanized anti-BCMA binding domain. In some embodiments, the cytoplasmic domain, or otherwise intracellular domain comprises a T-cell signaling domain, which is capable of transducing a signal, e.g., a T-cell activation signal, in the cell upon binding of the anti-BCMA CAR to a BCMA protein (e.g., a BCMA protein expressed on the surface of a cancer cell). In some embodiments, the T-cell signaling domain is capable of transducing a T-cell proliferation signal, a memory signal, a cytotoxic effector function signal, or a cytokine production signal. In some embodiments, the T-cell signaling domain comprises a costimulatory molecule. In some embodiments, the cytoplasmic domain further comprises a zeta chain portion. Exemplary T-cell signaling domains include, without limitation a CD8-alpha protein, a CD28 protein, a CD3 zeta protein, an FcR gamma protein, a CD27 protein, an OX40 protein, a 41BB protein, and any combination thereof. It should be appreciated, however, that the T-cell signaling domains provided herein are exemplary and the disclosure contemplates additional T-cell signaling domains, known or otherwise yet to be disclosed, that can be used in accordance with the invention.

In some embodiments, between the extracellular domain and the transmembrane domain of the CAR, or between the cytoplasmic domain and the transmembrane domain of the CAR, or between the BCMA-binding domain, there may be incorporated a spacer and/or hinge domain. As used herein, the term “spacer domain” generally refers to any oligo- or polypeptide that functions to link the transmembrane domain to, either the extracellular domain or, the cytoplasmic domain in the polypeptide chain. In some embodiments, a spacer domain may comprise up to 300 amino acids, e.g., 1 to 5 amino acids, 1 to 10 amino acids, 1 to 20 amino acids, 1 to 40 amino acids, 1 to 60 amino acids, 1 to 100 amino acids, 1 to 150 amino acids, 1 to 200 amino acids, 1 to 250 amino acids, 1 to 300 amino acids, 5 to 10 amino acids, 5 to 20 amino acids, 5 to 40 amino acids, 5 to 60 amino acids, 5 to 100 amino acids, 10 to 20 amino acids, 10 to 40 amino acids, 10 to 60 amino acids, 10 to 100 amino acids, 20 to 40 amino acids, 20 to 60 amino acids, 20 to 100 amino acids, 40 to 60 amino acids, 40 to 100 amino acids, or 60 to 100 amino acids. In some embodiments, a spacer domain may comprise 1, 2, 3, 4, 5, 6, 7, 8, 9, 10, 11, 12, 13, 14, 15, 16, 17, 18, 19, 20, 21, 22, 23, 24, or 25 amino acids. It also should be appreciated that one or more spacer domains may be included in other regions of a CAR, as aspects of the disclosure are not limited in this respect. Exemplary spacer sequences include, without limitation one or more of the following GGGGS (SEQ ID NO: 124); GGGGSGGGGS (SEQ ID NO: 125); (GGGGS)₃ (SEQ ID NO: 126); GSTSGGGSGGGSGGGGSS (SEQ ID NO: 127); GSTSGSGKPGSSEGSTKG (SEQ ID NO: 128); GGGGSGGG (SEQ ID NO: 129); and GGGS (SEQ ID NO: 130).

In some embodiments, any of the CARs provided herein comprise a hinge region, which typically connects the extracellular domain to the intracellular domain of a CAR. In some embodiments, the hinge region is between a BCMA-binding domain and a transmembrane domain of a CAR, or between a spacer domain and a transmembrane domain of the CAR. In some embodiments, the transmembrane domain can be attached to the extracellular domain of the CAR, e.g., the anti-BCMA binding domain of the CAR, via a hinge, e.g., a hinge from a human protein. For example, in one embodiment, the hinge can be from a human Ig (immunoglobulin) hinge, e.g., an IgG4 hinge, a CD8a hinge, or a CD28 hinge. In some embodiments, the hinge comprises an amino acid sequence of the hinge of a naturally-occurring Ig molecule, e.g., an IgG4 hinge, a CD8a hinge, or a CD28 hinge, or an amino acid sequence that is at least 75%, 80%, 85%, 90%, 95%, 96%, 97%, 98%, or 99% identical thereto. In some embodiments, the hinge comprises an amino acid sequence that is at least 75%, 80%, 85%, 90%, 95%, 96%, 97%, 98%, or 99% identical to FVPVFLPAKPTTTPAPRPPTPAPTIASQPLSLRPEACRPAAGGAVHTRGLDFACD (SEQ ID NO: 117). In some embodiments, the hinge comprises the amino acid sequence of SEQ ID NO: 117. However, it should be appreciated that these examples of hinges are not meant to be limiting and other hinges are contemplated. For Example, a hinge could also include one or more extracellular domains of a cell surface protein. See, e.g., Watanabe et al., “Fine-tuning the CAR spacer improves T-cell potency,” Oncoimmunology. 2016; 5(12): e1253656, the contents of which are hereby incorporated by reference.

In some embodiments, any of the CARs provided herein comprise the structure NH₂-[BCMA binding domain]-[transmembrane domain]-[cytoplasmic domain]-COOH. In some embodiments, the CAR comprises the structure NH₂-[BCMA binding domain]-[hinge region]-[transmembrane domain]-[cytoplasmic domain]-COOH. In some embodiments, the CAR comprises one or more spacer sequences. In some embodiments, each instance of “]-[” indicates the presence of an optional spacer sequence. In some embodiments, the cytoplasmic domain comprises a CD8-alpha protein, a CD28 protein, a CD3 zeta protein, an FcR gamma protein, a CD27 protein, an OX40 protein, a 41BB protein, and any combination thereof. In some embodiments, any of the CARs provided herein comprise the structure
[BCMA binding domain]-[transmembrane domain]-[cytoplasmic domain];
[BCMA binding domain]-[hinge region]-[transmembrane domain]-[cytoplasmic domain];
[Signal peptide]-[BCMA binding domain]-[transmembrane domain]-[cytoplasmic domain]; or
[Signal peptide]-[BCMA binding domain]-[hinge region]-[transmembrane domain]-[cytoplasmic domain];

In some embodiments, the CAR comprises a cytoplasmic domain having an arrangement selected from one of the following exemplary, non-limiting arrangements:
[CD3zeta];
[CD28]-[CD3zeta];
[41BB]-[CD3zeta];
[OX40]-[CD3zeta]; or
[41BB]-[OX40]-[CD3zeta]

In some embodiments, the above exemplary, non-limiting arrangements are from left to right, N-terminus to C-terminus of the CAR. In some embodiments, each instance of “]-[” indicates the presence of an optional space r sequence.

In one aspect, the anti-BCMA binding domain, e.g., human or humanized scFv, portion of a CAR of the invention is encoded by a transgene whose sequence has been codon optimized for expression in a mammalian cell. In one aspect, entire CAR construct of the invention is encoded by a transgene whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

The disclosure provides nucleic acid sequences coding for any of the molecules provided herein (e.g., anti-BCMA binding proteins, scFvs, CARS, and RNA constructs), which can be obtained using recombinant methods known in the art, such as, for example by screening libraries from cells expressing the gene, by deriving the gene from a vector known to include the same, or by isolating directly from cells and tissues containing the same, using standard techniques. Alternatively, the nucleic acid of interest can be produced synthetically, rather than cloned.

The present invention further includes retroviral and lentiviral vector constructs expressing a CAR that can be directly transduced into a cell.

The present invention also includes an RNA construct that can be directly transfected into a cell. One method for generating mRNA for use in transfection involves in vitro transcription (IVT) of a template with specially designed primers, optionally followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length but described in more detail hereunder. RNA so produced can efficiently transfect different kinds of cells. In one embodiment, the template includes sequences for the CAR. In an embodiment, an RNA CAR vector is transduced into a cell, e.g., T cell or NK cell, by electroporation.

### Anti-BCMA Binding Domains

In some embodiments, the CARs provided herein comprise an anti-B-Cell Maturation Antigen (BCMA) binding domain. In some embodiments, the CARs provided herein comprise a BCMA binding domain that is capable of binding to BCMA with greater affinity than other known anti-BCMA binding domains, for example an anti-BCMA binding domain from the CAR used in the clinical trial of Ali et al. (2016), i.e., of SEQ ID: 19. In some embodiments, any of the CARs of the invention can be engineered to target a tumor antigen of interest by way of engineering a desired BCMA binding moiety that specifically binds to a BCMA antigen on a tumor cell. In some embodiments, the anti-BCMA binding domains are N-terminus of to a transmembrane domain and/or intracellular domain of any of the CARs as described herein.

The BCMA binding domain can be any domain that binds to BCMA including but not limited to monoclonal antibodies, scFvs, polyclonal antibodies, synthetic antibodies, human antibodies, humanized antibodies, and antigen binding (e.g., BCMA-binding) fragments thereof. In some embodiments, it is beneficial for the BCMA binding domain to be humanized (e.g., partially humanized or fully humanized). In some instances, it is beneficial for the BCMA binding domain to be derived from the same species in which the CAR will ultimately be used in. For example, for use in humans, it may be beneficial for the BCMA binding domains of the CAR to comprise a human antibody or fragment thereof. Thus, in some embodiments, the BCMA binding domain portion comprises a human antibody or a fragment thereof. For in vivo use of antibodies in humans, it may be preferable to use human antibodies. Completely human antibodies are particularly desirable for therapeutic treatment of human subjects. Human antibodies can be made by a variety of methods known in the art including phage display methods using antibody libraries derived from human immunoglobulin sequences, including improvements to these techniques. See, also, U.S. Pat. Nos. 4,444,887 and 4,716,111; and PCT publications WO 98/46645, WO98/50433, WO 98/24893, WO98/16654, WO 96/34096, WO 96/33735, and WO91/10741; the contents of each of which is incorporated herein by reference in its entirety. A human antibody can also be an antibody wherein the heavy and light chains are encoded by a nucleotide sequence derived from one or more sources of human DNA. Human or humanized antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. For example, it has been described that the homozygous deletion of the antibody heavy chain joining region (JH) gene in chimeric and germ-line mutant mice results in complete inhibition of endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention.

In one aspect, the anti-BCMA binding domain is a fragment, e.g., a single chain variable fragment (scFv) or a fragment from an anti-BCMA antibody. In one aspect, the anti-BCMA binding domain is a Fv, a Fab, a (Fab′)2, or a bi-functional (e.g. bi-specific) hybrid antibody (e.g., Lanzavecchia et al., Eur. J. Immunol. 17, 105 (1987)). In one aspect, the antibodies and fragments thereof of the invention binds a BCMA protein with wild-type or enhanced affinity.

In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, the entire contents of each of which are incorporated by reference herein.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)n, where n is a positive integer equal to or greater than 1 (SEQ ID NO: 131). In one embodiment, the linker can be (Gly₄Ser)₄ (SEQ ID NO: 132) or (Gly₄Ser)₃ (SEQ ID NO: 133). In some embodiments, the scFv comprises a linker that is at least 70%, 75%, 80%, 85%, 90%, 95%, 96%, 97%, 98%, or 99% identical to the amino acid sequence of GSTSGSGKPGSGEGSTKG (SEQ ID NO: 108), or GSTSGSGKPGSGEGSTKGSGGGSGGG (SEQ ID NO:109). In some embodiments, the scFv comprises a linker that that comprises the amino acid sequence of SEQ ID NO: 108 or 109.

Antibodies directed against an antigen (e.g., BCMA) can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies, including, but not limited to, IgG1 (gamma 1) and IgG3. For a detailed discussion of this technology for producing human antibodies and human monoclonal antibodies and protocols for producing such antibodies, see, e.g., PCT Publication Nos. WO2014/055771, WO 98/24893, WO 96/34096, and WO 96/33735; and U.S. Pat. Nos. 5,413,923; 5,625,126; 5,633,425; 5,569,825; 5,661,016; 5,545,806; 5,814,318; and 5,939,598, each of which is incorporated by reference herein in their entirety. A “humanized” antibody retains a similar antigenic specificity as the original antibody, i.e., in the present invention, the ability to bind, for example, BCMA.

In some embodiments, antibodies (e.g., antibodies used to make BCMA binding domains), are tetrameric glycosylated proteins composed of two light (L) chains of approximately 25 kDa each and two heavy (H) chains of approximately 50 kDa each. Two types of light chain, termed lambda and kappa, may be found in antibodies. Depending on the amino acid sequence of the constant domain of heavy chains, immunoglobulins can be assigned to five major classes: A, D, E, G, and M, and several of these may be further divided into subclasses (isotypes), e.g., IgG₁, IgG₂, IgG₃, IgG₄, IgA₁, and IgA₂. Each light chain typically includes an N-terminal variable (V) domain (V_{L}) and a constant (C) domain (C_{L}). Each heavy chain typically includes an N-terminal V domain (V_{H}), three or four C domains (C_{H}1-3), and a hinge region. The Ca domain most proximal to V_{H} is designated as C_{H}1. The V_{H} and V_{L} domains consist of four regions of relatively conserved sequences called framework regions (FR1, FR2, FR3, and FR4), which form a scaffold for three regions of hypervariable sequences (complementarity determining regions, CDRs). The CDRs contain most of the residues responsible for specific interactions of the antibody with the antigen. CDRs are referred to as CDR1, CDR2, and CDR3. Accordingly, CDR constituents on the heavy chain are referred to as CDRH1, CDRH2, and CDRH3, while CDR constituents on the light chain are referred to as CDRL1, CDRL2, and CDRL3. The CDRs typically refer to the Kabat CDRs, as described in Sequences of Proteins of Immunological Interest, US Department of Health and Human Services (1991), eds. Kabat et al. Another standard for characterizing the antigen binding site is to refer to the hypervariable loops as described by Chothia. See, e.g., Chothia, D. et al. (1992) J. Mol. Biol. 227:799-817; and Tomlinson et al. (1995) EMBO J. 14:4628-4638. Still another standard is the AbM definition used by Oxford Molecular's AbM antibody modeling software. See, generally. e.g., Protein Sequence and Structure Analysis of Antibody Variable Domains. In: Antibody Engineering Lab Manual (Ed.: Duebel, S, and Kontermann, R., Springer-Verlag, Heidelberg). Embodiments described with respect to Kabat CDRs can alternatively be implemented using similar described relationships with respect to Chothia hypervariable loops or to the AbM-defined loops, or combinations of any of these methods.

CAR T cells provided herein are capable of binding to BCMA with greater affinity and/or more efficiently kill-BCMA expressing cells (e.g., myeloma cells) than other CAR T cells known in the art, e.g., the CAR T cells used in the clinical trial of Ali et al. (2016), i.e., of a CAR of SEQ ID: 19. In some embodiments, anti-BCMA binding domains provided herein can bind to BCMA with an affinity that is at least 5%, 10%, 15%, 20%, 30%, 40%, 50%, 60%, 70%, 80%, 90%, 100%, 120%, 140%, 160%, 180%, 200%, 250%, 300%, 350%, 400%, 450%, 500%, 600%, 700%, 800%, 900%, or 1,000% greater than the affinity of another anti-BCMA binding domain to BCMA, e.g., the BCMA binding domain from the CAR used in the clinical trial of Ali et al. (2016), i.e., of SEQ ID: 19. In some embodiments, anti-BCMA binding domains provided herein, when comprised in a CAR expressed on a T cell, can kill cancer cells (e.g., BCMA expressing myeloma cells) more efficiently (e.g., at least 5%, 10%, 15%, 20%, 30%, 40%, 50%, 60%, 70%, 80%, 90%, 100%, 120%, 140%, 160%, 180%, 200%, 250%, 300%, 350%, 400%, 450%, 500%, 600%, 700%, 800%, 900%, or 1,000% more efficiently) than another anti-BCMA CAR, e.g., the CAR used in the clinical trial of Ali et al. (2016), i.e., of SEQ ID: 19. Exemplary CDR sequences used in anti-BCMA binding domains are provided below. Those amino acid residues that are different with respect to the reference CDR sequences (i.e., SEQ ID NOs: 1, 4, 7, 10, 12, and 16) are indicated by bold and underlining.

CDRH1:
(SEQ ID NO: 1)
DYSIN;
(SEQ ID NO: 2)
SYSIN;
(SEQ ID NO: 3)
SYDIN
CDRH2:
(SEQ ID NO: 4)
WINTETREPAYAYDFRG;
(SEQ ID NO: 5)
WINTNTGNPTYAQGFTG;
(SEQ ID NO: 6)
WINTETREPAYAQGFTG
CDRH3:
(SEQ ID NO: 7)
DYSYAMDY;
(SEQ ID NO: 8)
DYTYGMDY;
(SEQ ID NO: 9)
DYLYSLDF
CDRL1:
(SEQ ID NO: 10)
RASESVTILGSHLIH;
(SEQ ID NO: 11)
RASESVSFLGINLIH
CDRL2:
(SEQ ID NO: 12)
QLASNVQT;
(SEQ ID NO: 13)
YLASNLET;
(SEQ ID NO: 14)
YSASNLQS;
(SEQ ID NO: 15)
NLASNVNT
CDRL3:
(SEQ ID NO: 16)
LQSRTIPRT;
(SEQ ID NO: 17)
LQSRTLPRT;
(SEQ ID NO: 18)
LQSKNFPRT

In some embodiments, the anti-BCMA binding domain comprises at least one (e.g., at least 2, 3, 4, 5, or 6 CDR sequences) CDR sequence selected from any one of SEQ ID NOs: 2, 3, 5, 6, 8, 9, 11, 13, 14, 15, 17, and 18. In some embodiments, the anti-BCMA binding domain comprises at least one of the CDRH1 amino acid sequences of any one of SEQ ID NOs: 1-3. In some embodiments, the anti-BCMA binding domain comprises at least one of the CDRH2 amino acid sequences of any one of SEQ ID NOs: 4-6. In some embodiments, the anti-BCMA binding domain comprises at least one of the CDRH3 amino acid sequences of any one of SEQ ID NOs: 7-9. In some embodiments, the anti-BCMA binding domain comprises at least one of the CDRL1 amino acid sequences of any one of SEQ ID NOs: 10-11. In some embodiments, the anti-BCMA binding domain comprises at least one of the CDRL2 amino acid sequences of any one of SEQ ID NOs: 12-15. In some embodiments, the anti-BCMA binding domain comprises at least one of the CDRL3 amino acid sequences of any one of SEQ ID NOs: 16-18. In some embodiments, the anti-BCMA binding domain comprises the CDRH3 of SEQ ID NO: 8 or 9. In some embodiments, the anti-BCMA binding domain comprises the CDRL3 of SEQ ID NO: 17 or 18.

In some embodiments, the anti-BCMA binding domain includes a CDRH1, CDRH2, CDRH3, CDRL1, CDRL2, or CDRL3, or combinations thereof, as provided for any one of the scFv clones shown in Table 1. In some embodiments, anti-BCMA binding domains include the CDRH1, CDRH2, CDRH3, CDRL1, CDRL2, and CDRL3 of any one of the scFv clones shown in Table 1. The disclosure also includes any nucleic acid sequence that encodes a molecule comprising a CDRH1, CDRH2, CDRH3, CDRL1, CDRL2, or CDRL3 as provided for any one of the scFv clones shown in Table 1. Antibody heavy and light chain CDR3 domains may play a particularly important role in the binding specificity/affinity of an antibody for an antigen. Accordingly, the anti-BCMA binding domains of the disclosure, may include at least the heavy and/or light chain CDR3s of antibodies as shown in Table 1. Aspects of the disclosure relate to anti-BCMA biding domains that bind to BCMA protein (e.g., a BCMA protein on a tumor cell) and that comprises six complementarity determining regions (CDRs): CDRH1, CDRH2, CDRH3, CDRL1, CDRL2, and CDRL3.

TABLE 1
Combination of CDR sequences (CDRH 1-3 and CDRL 1-3) for each scFv used in the CARs
provided herein. The parent framework region used for each scFv is also indicated by the
parent framework of the indicated SEQ ID NOs. Each CDR is indicated by their SEQ ID NO.
Those SEQ ID NOs marked with an “*” are different from the reference CDR sequences
of SEQ ID NOs: 1, 4, 7, 10, 12, and 16.

| | Parent | CDRH 1 | CDRH 2 | CDRH 3 | CDRL 1 | CDRL 2 | CDRL 3 |
|---|---|---|---|---|---|---|---|
| New | Framework | SEQ | SEQ | SEQ | SEQ | SEQ | SEQ |
| scFv | SEQ ID NO: | ID NO: | ID NO: | ID NO: | ID NO: | ID NO: | ID NO: |
| R1 | 48 | 1 | 4 | 7 | 10 | 12 | 16 |
| Y32 | 57 | 1 | 4 | 7 | 10 | 12 | 16 |
| Y37 | 60 | 1 | 4 | 7 | 10 | 12 | 16 |
| 1′ | 57 | 3* | 4 | 7 | 10 | 12 | 16 |
| 4′ | 57 | 1 | 4 | 8* | 10 | 12 | 16 |
| 8′ | 57 | 1 | 4 | 7 | 11* | 14* | 17* |
| 9′ | 57 | 1 | 4 | 7 | 11* | 14* | 18* |
| 10′ | 57 | 2* | 6* | 9* | 10 | 12 | 16 |
| 11′ | 57 | 3* | 5* | 8* | 10 | 12 | 16 |
| 12′ | 57 | 3* | 4 | 9* | 10 | 12 | 18* |
| 13′ | 57 | 2* | 6* | 9* | 11* | 13* | 17* |
| 21′ | 57 | 1 | 4 | 7 | 10 | 15* | 16 |
| K′ | 60 | 2* | 6* | 9* | 11* | 14* | 17* |
| R′ | 60 | 2* | 6* | 9* | 11* | 14* | 17* |
| J′ | 60 | 2* | 6* | 9* | 11* | 14* | 18* |
| L′ | 60 | 2* | 6* | 9* | 11* | 14* | 18* |
| O′ | 60 | 1 | 4 | 8* | 10 | 15* | 17* |
| S′ | 60 | 1 | 4 | 8* | 10 | 15* | 17* |

In some embodiments, the anti-BCMA binding domain comprises six complementarity determining regions (CDRs): CDRH1, CDRH2, CDRH3, CDRL1, CDRL2, and CDRL3. In some embodiments, the anti-BCMA binding domain comprises any one of the following groups of CDRs, which are listed in the order of CDRH1, CDRH2, CDRH3, CDRL1, CDRL2, and CDRL3:
SEQ ID NOs: 3, 4, 7, 10, 12, and 16;
SEQ ID NOs: 1, 4, 8, 10, 12, and 16;
SEQ ID NOs: 1, 4, 7, 11, 14, and 17;
SEQ ID NOs: 1, 4, 7, 11, 14, and 18;
SEQ ID NOs: 2, 6, 9, 10, 12, and 16;
SEQ ID NOs: 3, 5, 8, 10, 12, and 16;
SEQ ID NOs: 3, 4, 9, 10, 12, and 18;
SEQ ID NOs: 2, 6, 9, 11, 13, and 17;
SEQ ID NOs: 1, 4, 7, 10, 15, and 16;
SEQ ID NOs: 2, 6, 9, 11, 14, and 17;
SEQ ID NOs: 2, 6, 9, 11, 14, and 18;
SEQ ID NOs: 1, 4, 8, 10, 15, and 17; and
SEQ ID NOs: 1, 4, 7, 10, 12, and 16.

It should be appreciated that the disclosure contemplates any of the CDR sequences provided herein (e.g., SEQ ID NOs: 1-18) having 1, 2, or 3, but no more than three conservative mutations. Accordingly, in some embodiments, any of the CDR sequences provided herein, i.e., any of the CDH1, CDH2, CDH3, CDL1, CDL2, or CDL3, CDR sequences provided herein, may comprise 1, 2 or 3 conservative mutations. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-BCMA binding domain comprises a heavy chain variable domain and/or a light chain variable domain. In some embodiments, the anti-BCMA binding domain comprises a heavy chain variable domain framework sequence that is at least 75% (e.g., 80%, 85%, 90%, 95%, 98%, or 99%) identical to the heavy chain variable domain framework sequence from the scFv of any one of SEQ ID NOs: 48-72 or a light chain variable domain framework sequence from the scFv of any one of SEQ ID NOs: 48-72. In some embodiments, the homologous heavy chain variable domain framework sequence and/or a light chain variable domain framework sequence do not vary within any of the CDR sequences provided herein. For example, in some embodiments, the degree of sequence variation (e.g., 75%, 80%, 85%, 90%, 95%, 98%, or 99%) may occur within a heavy chain variable and/or a light chain variable sequence excluding any of the CDR sequences provided herein. In the scFv amino acid sequences provided below (SEQ ID NOs 48-72), the heavy chain variable sequence is indicated in bold, the light chain variable sequence is indicated in italics, and the linker sequence is indicated by underlining. In some embodiments, the anti-BCMA binding domain comprises a single chain variable fragment (scFv) made up of a variable heavy chain (VH) and a variable light chain (VL). In some embodiments, the anti-BCMA binding domain comprises the amino acid sequence of any one of SEQ ID NOs 48-72

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, word length=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

scFv-R1
(SEQ ID NO: 48)
DIVLTQSPPSLAMSLGKRATISCRASESVHLGSHLIHWYQQKPGQPPTLLIQLASNVQTGVP
ARFSGSGSRTDFTLTIDPVEEDDVAVYYCLQSRTIPRTFGGGTKLEIKGSTSGSGKPGSGE
GSTKGQIQLVQSGPELKKPGETVKISCKASGYTFTDYSINWVKRAPGKGLKWM
GWINTETREPAYAYDFRGRFAFSLETSASTAYLQINNLKYEDTATYFCALDYSYA
MDYWGQGTSVTVSS
scFv-J1
(SEQ ID NO: 49)
DIVMTQSPDSLSVSLGERATINCRASESVTILGSHLIHWYQQKPGQPPKLLIQLASNVQTGV
PDRFSGSGSGTDFTLTISSLQAEDVAVYYCLQSRTIPRTFGGGTKVEIKGSTSGSGKPGSGE
GSTKGQIQLVQSGAEVKKPGASVKISCKASGYTFTDYSINWVRQAPGQGLEWM
GWINTETREPAYAYDFRGRVTMTRDTSASTAYLQISSLKAEDTAVYFCALDYSYA
MDYWGQGSLVTVSS
scFv-J2
(SEQ ID NO: 50)
DIVLTQSPASLAVSLGERATISCRASESVSILGSHLLHWYQQKPGQPPKLLIYLASNLQTGVPA
RFSGSGSGTDFTLTISSLEAEDVAVYYCLQSRTIPRTFGQGTKLEIKGSTSGSGKPGSGEGS
TKGQIQLVQSGAEVKKPGASVKISCKASGYTFTDYSINWVRQAPGQGLEWMGW
INTETREPAYAYDFRGRVTMTRDTSASTAYLQISSLKAEDTAVYFCALDYSYAMD
YWGQGSLVTVSS
scFv-J3
(SEQ ID NO: 51)
DIVLTQSPASLAVSLGKRATISCRASESVHLGSHLIHWYQQKPGQPPKLLIYLASNVQTGVPA
RFSGSGSGTDFTLTISSLEAEDVAVYYCLQSRTIPRTFGQGTKLEIKGSTSGSGKPGSGEGS
TKGQVQLVQSGAEVKKPGASVKISCKASGYTFTDYSINWVRQAPGQGLEWMG
WINTETREPAYAYDFRGRVTMTRDTSASTAYLQISSLKAEDTAVYFCALDYSYAM
DYWGQGSLVTVSS
scFv-J4
(SEQ ID NO: 52)
DIVMTQSPDSLSVSLGERATINCRASESVTILGSHLIHWYQQKPGQPPKLLIQLASNVQTGV
PDRFSGSGSGTDFTLTISSLQAEDVAVYYCLQSRTIPRTFGGGTKVEIKGSTSGSGKPGSGE
GSTKGQIQLVQSGAEVKKPGESVKISCKASGYTFTDYSINWVRQAPGQGLKWM
GWINTETREPAYAYDFRGRFAFSLDTSASTAYLQISSLKAEDTAVYFCALDYSYAM
DYWGQGTLVTVSS
scFv-J5
(SEQ ID NO: 53)
DIVLTQSPASLAVSLGERATISCRASESVSILGSHYLAWYQQKPGQPPKLLIYLASNLQTGVPA
RFSGSGSGTDFTLTISSLEAEDVAVYYCQQSRTIPRTFGQGTKLEIKGSTSGSGKPGSGEGS
TKGQIQLVQSGAEVKKPGESVKISCKASGYTFTDYSINWVRQAPGQGLKWMGW
INTETREPAYAYDFRGRFAFSLDTSASTAYLQISSLKAEDTAVYFCALDYSYAMDY
WGQGTLVTVSS
scFv-Yi1
(SEQ ID NO: 54)
DIVLTQSPSSLSASVGDRATISCRASESVHLGSHLIHWYQQKPGQAPKLLIQLASNVQTGVP
ARFSGSGSGTDFTLTISSVEPDDVAVYYCLQSRTIPRTFGGGTKLEIKGSTSGSGKPGSGEG
STKGQIQLVQSGPELKKPGGSVKISCAASGYTFTDYSINWVKQAPGKGLEWVG
WINTETREPAYAYDFRGRFTFSLDTSKSTAYLQMNSLRAEDTAVYYCALDYSYAM
DYWGQGTLVTVSS
scFv-Yi2
(SEQ ID NO: 55)
DIQLTQSPSSLSASVGDRVTITCRASQSVTILGSHLIHWYQQKPGKAPKLLIQLASNVQTGVP
SRFSGSGSGTDFTLTISSLQPEDVATYYCLQSRTIPRTFGQGTKLEIKGSTSGSGKPGSGEG
STKGQVQLVQSGGGLVQPGRSVKLSCAASGYTFTDYSINWVRQAPGKGLEWVG
WINTETREPAYAYDFRGRFTISRDTSKNTAYLQMNSLRAEDTAVYYCARDYSYAM
DYWGQGTLVTVSS
scFv-Y31
(SEQ ID NO: 56)
DIVLTQSPSSLSASVGDRATISCRASESVHLGSHLIHWYQQKPGQAPKLLIQLASNVQTGVP
ARFSGSGSGTDFTLTISSVEPEDVAVYYCLQSRTIPRTFGQGTKVEIKGSTSGSGKPGSGEG
STKGQIQLVQSGPELKKPGGSVKISCAASGYTFTDYSINWVKQAPGKGLEWVG
WINTETREPAYAYDFRGRFTFSADTSKSTAYLQMNSLRAEDTAVYYCALDYSYAM
DYWGQGTLVTVSS
scFv-Y32
(SEQ ID NO: 57)
DIVLTQSPSSLSASVGDRATISCRASESVHLGSHLIHWYQQKPGQAPKLLIQLASNVQTGVP
ARFSGSGSGTDFTLTISSVEPEDVAVYYCLQSRTIPRTFGQGTKVEIKGSTSGSGKPGSGEG
STKGQIQLVQSGPELKKPGGSVKISCAASGYTFTDYSINWVRQAPGKGLEWVG
WINTETREPAYAYDFRGRFTFSADTSKSTAYLQMNSLRAEDTAVYYCALDYSYAM
DYWGQGTLVTVSS
scFv-Y33
(SEQ ID NO: 58)
DIVLTQSPSSLSASVGDRATISCRASESVHLGSHLIHWYQQKPGQAPKLLIQLASNVQTGVP
ARFSGSGSGTDFTLTISSVEPEDVAVYYCLQSRTIPRTFGQGTKVEIKGSTSGSGKPGSGEG
STKGQIQLVESGAEVKKPGGSVKISCAASGYTFTDYSINWVKQAPGKGLEWVG
WINTETREPAYAYDFRGRFTFSLDTSKSTAYLQMNSLRAEDTAVYYCALDYSYAM
DYWGQGTLVTVSS
scFv-Y34
(SEQ ID NO: 59)
DIVLTQSPSSLSASVGDRATISCRASESVHLGSHLIHWYQQKPGQAPKLLIQLASNVQTGVP
ARFSGSGSGTDFTLTISSVEPEDVAVYYCLQSRTIPRTFGQGTKVEIKGSTSGSGKPGSGEG
STKGQIQLVESGAEVKKPGGSVKVSCKASGYTFTDYSINWVRQAPGKGLEWVG
WINTETREPAYAYDFRGRFAISAETSKSTAYLQMNSLRAEDTAVYYCALDYSYAM
DYWGQGTLVTVSS
scFv-Y37
(SEQ ID NO: 60)
DIVLTQSPASLAVSPGQRATITCRASESVTILGSHLIHWYQQKPGQPPKLLIQLASNVQTGVP
ARFSGSGSGTDFTLTISSVEPEDTANYYCLQSRTIPRTFGQGTKVEIKGSTSGSGKPGSGEG
STKGQIQLVQSGPELKKPGGSVKISCKASGYTFTDYSINWVRQAPGKGLEWVG
WINTETREPAYAYDFTGRFTFSADTSKSMAYLQINSLRAEDTAVYYCALDYSYAM
DYWGQGTLVTVSS
scFv-CAS2 (C2)
(SEQ ID NO: 61)
DIVLTQSPASLAVSPGQRATISCRASESVTILGSHLIHWYQQKPGQPPKLLIQLASNVQTGVP
ARFSGSGSRTDFTLTISSLEPEDVAVYYCLQSRTIPRTFGQGTKVEIKGSTSGSGKPGSGEG
STKGQITLKESGPTLVKPTQTLTLSCKASGYTFTDYSINWVRRAPGKGLEWMG
WINTETREPAYAYDFRGRFVFSLDTSVSMAYLQISSLKAEDTAVYYCALDYSYAM
DYWGQGTLVTVSS
scFv-1′
(SEQ ID NO: 62)
DIVLTQSPSSLSASVGDRATISCRASESVHLGSHLIHWYQQKPGQAPKLLIQLASNVQTGVP
ARFSGSGSGTDFTLTISSVEPEDVAVYYCLQSRTIPRTFGQGTKVEIKGSTSGSGKPGSGEG
STKGQIQLVQSGPELKKPGGSVKISCAASGYTFTSYDINWVRQAPGKGLEWVG
WINTETREPAYAYDFRGRFTFSADTSKSTAYLQMNSLRAEDTAVYYCALDYSYAM
DYWGQGTLVTVSS
scFv-4′
(SEQ ID NO: 63)
DIVLTQSPSSLSASVGDRATISCRASESVHLGSHLIHWYQQKPGQAPKLLIQLASNVQTGVP
ARFSGSGSGTDFTLTISSVEPEDVAVYYCLQSRTIPRTFGQGTKVEIKGSTSGSGKPGSGEG
STKGQIQLVQSGPELKKPGGSVKISCAASGYTFTDYSINWVRQAPGKGLEWVG
WINTETREPAYAYDFRGRFTFSADTSKSTAYLQMNSLRAEDTAVYYCALDYTYG
MDYWGQGTLVTVSS
scFv-8′
(SEQ ID NO: 64)
DIVLTQSPSSLSASVGDRATISCRASESVSFLGINLIHWYQQKPGQAPKLLIYSASNLQSGVPA
RFSGSGSGTDFTLTISSVEPEDVAVYYCLQSRTLPRTFGQGTKVEIKGSTSGSGKPGSGEGS
TKGQIQLVQSGPELKKPGGSVKISCAASGYTFTDYSINWVRQAPGKGLEWVGWI
NTETREPAYAYDFRGRFTFSADTSKSTAYLQMNSLRAEDTAVYYCALDYSYAMDY
WGQGTLVTVSS
scFv-9′
(SEQ ID NO: 65)
DIVLTQSPSSLSASVGDRATISCRASESVSFLGINLIHWYQQKPGQAPKLLIYSASNLQSGVPA
RFSGSGSGTDFTLTISSVEPEDVAVYYCLQSKNFPRTFGQGTKVEIKGSTSGSGKPGSGEG
STKGQIQLVQSGPELKKPGGSVKISCAASGYTFTDYSINWVRQAPGKGLEWVG
WINTETREPAYAYDFRGRFTFSADTSKSTAYLQMNSLRAEDTAVYYCALDYSYAM
DYWGQGTLVTVSS
scFv-10′
(SEQ ID NO: 66)
DIVLTQSPSSLSASVGDRATISCRASESVHLGSHLIHWYQQKPGQAPKLLIQLASNVQTGVP
ARFSGSGSGTDFTLTISSVEPEDVAVYYCLQSRTIPRTFGQGTKVEIKGSTSGSGKPGSGEG
STKGQIQLVQSGPELKKPGGSVKISCAASGYTFTSYSINWVRQAPGKGLEWVGW
INTETREPAYAQGFTGRFTFSADTSKSTAYLQMNSLRAEDTAVYYCALDYLYSLD
FWGQGTLVTVSS
scFv-11′
(SEQ ID NO: 67)
DIVLTQSPSSLSASVGDRATISCRASESVHLGSHLIHWYQQKPGQAPKLLIQLASNVQTGVP
ARFSGSGSGTDFTLTISSVEPEDVAVYYCLQSRTIPRTFGQGTKVEIKGSTSGSGKPGSGEG
STKGQIQLVQSGPELKKPGGSVKISCAASGYTFTSYDINWVRQAPGKGLEWVG
WINTNTGNPTYAQGFTGRFTFSADTSKSTAYLQMNSLRAEDTAVYYCALDYTYG
MDYWGQGTLVTVSS
scFv-12′
(SEQ ID NO: 68)
DIVLTQSPSSLSASVGDRATISCRASESVHLGSHLIHWYQQKPGQAPKLLIQLASNVQTGVP
ARFSGSGSGTDFTLTISSVEPEDVAVYYCLQSKNFPRTFGQGTKVEIKGSTSGSGKPGSGE
GSTKGQIQLVQSGPELKKPGGSVKISCAASGYTFTSYDINWVRQAPGKGLEWVG
WINTETREPAYAYDFRGRFTFSADTSKSTAYLQMNSLRAEDTAVYYCALDYLYSL
DFWGQGTLVTVSS
scFv-13′
(SEQ ID NO: 69)
DIVLTQSPSSLSASVGDRATISCRASESVSFLGINLIHWYQQKPGQAPKLLIYLASNLETGVPA
RFSGSGSGTDFTLTISSVEPEDVAVYYCLQSRTLPRTFGQGTKVEIKGSTSGSGKPGSGEGS
TKGQIQLVQSGPELKKPGGSVKISCAASGYTFTSYSINWVRQAPGKGLEWVGWI
NTETREPAYAQGFTGRFTFSADTSKSTAYLQMNSLRAEDTAVYYCALDYLYSLDF
WGQGTLVTVSS
scFv-21′
(SEQ ID NO: 70)
DIVLTQSPSSLSASVGDRATISCRASESVHLGSHLIHWYQQKPGQAPKLLINLASNVNTGVP
ARFSGSGSGTDFTLTISSVEPEDVAVYYCLQSRTIPRTFGQGTKVEIKGSTSGSGKPGSGEG
STKGQIQLVQSGPELKKPGGSVKISCAASGYTFTDYSINWVRQAPGKGLEWVG
WINTETREPAYAYDFRGRFTFSADTSKSTAYLQMNSLRAEDTAVYYCALDYSYAM
DYWGQGTLVTVSS
scFv-2′
(SEQ ID NO: 71)
DIVLTQSPSSLSASVGDRATISCRASESVITLGSHLIHWYQQKPGQAPKLLIQLASNVQTGVP
ARFSGSGSGTDFTLTISSVEPEDVAVYYCLQSRTLPRTFGQGTKVEIKGSTSGSGKPGSGEG
STKGQIQLVQSGPELKKPGGSVKISCAASGYTFTDYSINWVRQAPGKGLEWVG
WINTETREPAYAYDFRGRFTFSADTSKSTAYLQMNSLRAEDTAVYYCALDYSYAM
DYWGQGTLVTVSS
scFv-3′
(SEQ ID NO: 72)
DIVLTQSPSSLSASVGDRATISCRASESVITLGSHLIHWYQQKPGQAPKLLIQLASNVQTGVP
ARFSGSGSGTDFTLTISSVEPEDVAVYYCLQSRTLPRTFGQGTKVEIKGSTSGSGKPGSGEG
STKGQIQLVQSGPELKKPGGSVKISCAASGYTFTDYSINWVRQAPGKGLEWVG
WINTETREPAYAYDFRGRFTFSADTSKSTAYLQMNSLRAEDTAVYYCALDYSYAM
DYWGQGTLVTVSS

### Leader Domains

In some embodiments, a CAR is designed with a leader domain (also referred to as a “signal peptide”) for directing the translated chimeric protein to the membrane. In some embodiments the CAR comprises a leader sequence at the amino-terminus (N-ter) of the CAR protein. In one aspect, the CAR further comprises a leader sequence at the N-terminus of the extracellular BCMA binding domain, wherein the leader sequence is optionally cleaved from the BCMA binding domain (e.g., scFv) during cellular processing and localization of the CAR to the cellular membrane. The leader domain is generally in the range of 15 to 30 amino acids. Examples of leader domains include a CD8a leader (21 amino acids), a CD33 leader (17 amino acids), a CD4 leader (25 amino acids), a IL-2R (CD25) leader (21 amino acids), a trypsinogen-2 leader (15 amino acids), a VEGFR1 leader (26 amino acids), a EGFR leader (24 amino acids) a GMCSFR leader (22 amino acids), a IgVL leader, a IgVK leader, or a Ig VH leader. In some embodiments, the leader sequence is at least 80%, 90%, 95%, 96%, 97%, 98%, or 99% identical to any of the leader domains provided herein. In some embodiments, the leader sequence is at least 80%, 85%, 90%, 95%, 96%, 97%, 98%, or 99% identical to the amino acid sequence of MALPVTALLLPLALLLHAARP (SEQ ID NO: 110). In some embodiments, the leader sequence comprises the amino acid sequence of SEQ ID NO: 110.

### Transmembrane and Hinge Domains

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one aspect, the transmembrane domain is one that is associated with one of the other domains of the CAR is used. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another CAR on the CAR-expressing cell, e.g., CAR T cell, surface. In a different aspect the amino acid sequence of the transmembrane domain may be modified or substituted so as to minimize interactions with the binding domains of the native binding partner present in the same CAR-expressing cell, e.g., CAR T cell.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the CAR has bound to a target. A transmembrane domain of particular use in this invention may include at least the transmembrane region(s) of e.g., the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37. CD64, CD80, CD86, CD134, CD137, CD154. In some embodiments, a transmembrane domain may include at least the transmembrane region(s) of a costimulatory molecule, e.g., MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGLI, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. Transmembrane domains can be identified using any method known in the art or described herein, e.g., by using the UniProt Database.

In some embodiments, the transmembrane domain may be synthetic, in which case it will comprise predominantly hydrophobic residues such as leucine and valine. In some embodiments, a triplet of phenylalanine, tryptophan and valine will be found at each end of a synthetic transmembrane domain. Optionally, a short oligo- or polypeptide linker, e.g., between 2 and 10 amino acids in length may form the linkage between the transmembrane domain and the cytoplasmic signaling domain of the CAR. A glycine-serine doublet provides an exemplary suitable linker.

In some embodiments, the transmembrane domain in the CAR of the invention is a CD8 transmembrane domain. Sequences of CD8 for this purpose are taught in PCT Pub No. WO2014/055771, which is incorporated by reference herein. In some embodiments, the transmembrane domain in the CAR is a CD8a transmembrane. In some embodiments, the transmembrane domain comprises the amino acid sequence of IYIWAPLAGTCGVLLLSLVIT (SEQ ID NO 118), IYIWAPLAGTCGVLLLSLVITLYCNHRN (SEQ ID NO: 120), or an amino acid sequence that is at least 70%, 75%, 80%, 85%, 90%, 95%, 96%, 97%, 98%, or 99% identical thereto. It should be appreciated that a portion of the transmembrane domain may be extracellular or cytosolic, for example, in some embodiments, the transmembrane domain comprises a cytosolic portion comprising the amino acid sequence of LYCNHRN (SEQ ID NO: 119).

In some embodiments, the transmembrane domain in the CAR of the invention is the CD28 transmembrane domain. One skilled in the art would appreciate that the full transmembrane domain, or portion thereof, is implemented with the cytoplasmic domain, or a portion thereof. Typically, the transmembrane and cytoplasmic domains used would be contiguous portions of the CD28 sequence. In some embodiments, the CD28 transmembrane domain comprises a fragment or variant thereof that is capable of anchoring a CAR comprising the sequence to a cell membrane.

In some instances, the transmembrane domain is attached to the extracellular region of the CAR, e.g., the antigen binding domain of the CAR, via a hinge, e.g., a hinge from a human protein. For example, in one embodiment, the hinge can be a human Ig (immunoglobulin) hinge, e.g., an IgG4 hinge, or a CD8a hinge. In one embodiment, the hinge or spacer comprises (e.g., consists of) the amino acid sequence of FVPVFLPAKPTTTPAPRPPTPAPTIASQPLSLRPEACRPAAGGAVHTRGLDFACD (SEQ ID NO: 117), or an amino acid sequence that is at least 70%, 75%, 80%, 85%, 90%, 95%, 96%, 97%, 98%, or 99% identical thereto. In one aspect, the transmembrane and hinge domains comprise (e.g., consists of) the amino acid sequence of FVPVFLPAKPTTTPAPRPPTPAPTIASQPLSLRPEACRPAAGGAVHTRGLDFACDIYIWA PLAGTCGVLLLSLVITLYCNHRN (SEQ ID NO: 111).

### Cytoplasmic Domain

Some aspects of the disclosure provide CARs having a cytoplasmic domain (also referred to as an intracellular domain or intracellular signaling domain). The cytoplasmic domain or region of a CAR of the present invention includes an intracellular signaling domain. An intracellular signaling domain is generally responsible for activation of at least one of the normal effector functions of the immune cell in which the CAR has been introduced. In some embodiments, the cytoplasmic domain of the CAR is responsible for activation of at least one of the normal effector functions of the immune cell in which the CAR has been introduced in. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire domain. To the extent that a truncated portion of the intracellular signaling domain is used, such truncated portion may be used in place of the intact domain as long as it transduces the effector function signal. The term intracellular signaling domain is thus meant to include any truncated portion of the intracellular signaling domain sufficient to transduce the effector function signal.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs known as immunoreceptor tyrosine-based activation motifs or ITAMs.

Examples of ITAM containing primary intracellular signaling domains that are of particular use in the invention include those of TCR zeta, FcR gamma, FcR beta, CD3 gamma, CD3 delta, CD3 epsilon, CD5, CD22, CD79a, CD79b, CD278 (also known as “ICOS”), Fc.epsilon.RI, DAP10, DAP12, and CD66d. In one embodiment, a CAR of the invention comprises an intracellular signaling domain, e.g., a primary signaling domain of CD3zeta. An exemplary CD3zeta signaling domain is provided below.

In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs. Further examples of molecules containing a primary intracellular signaling domain include those of DAP10, DAP12, and CD32.

The intracellular signaling domain of the CAR can comprise a primary signaling domain, e.g., CD3-zeta signaling domain, by itself or it can be combined with any other desired intracellular signaling domain(s) useful in the context of a CAR of the invention. For example, the intracellular signaling domain of the CAR can comprise a primary signaling domain, e.g., CD3 zeta chain portion, and one or more costimulatory signaling domains. The costimulatory signaling domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGLI, CD100 (SEMA4D), CD69, SLAMF6 (NTB-A, Ly 108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, and the like. For example, CD27 costimulation has been demonstrated to enhance expansion, effector function, and survival of human CAR T cells in vitro and augments human T cell persistence and antitumor activity in vivo (Song et al. Blood. 2012; 119(3):696-706). The intracellular signaling sequences within the cytoplasmic portion of the CAR of the invention may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequence. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. Exemplary costimulatory domains are provided below. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue.

### Exemplary CD3zeta Signaling Domain:

(SEQ ID NO: 107)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

### Exemplary Costimulatory Domains:

CD28
(SEQ ID NO: 100)
RSKRSRLLHSDYMNMTPRRPGPTRKHYQPYAPPRDFAAYRS
41BB
(SEQ ID NO: 101)
KRGRKKLLYIFKQPFMRPVQTTQEEDGCSCRFPEEEEGGCEL
OX40
(SEQ ID NO: 102)
RRDQRLPPDAHKPPGGGSFRTPIQEEQADAHSTLAKI

### Exemplary Combinations of Signaling and Costimulatory Domains

CD28-CD3zeta
(SEQ ID NO: 103)
RSKRSRLLHSDYMNMTPRRPGPTRKHYQPYAPPRDFAAYRSRVKFSRSA
DAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHM
QALPPR
41BB-CD3zeta
(SEQ ID NO: 104)
KRGRKKLLYIFKQPFMRPVQTTQEEDGCSCRFPEEEEGGCELRVKFSRS
ADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALH
MQALPPR
OX40-CD3zeta
(SEQ ID NO: 105)
RRDQRLPPDAHKPPGGGSFRTPIQEEQADAHSTLAKIRVKFSRSADAPA
YQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALP
PR
41BB-OX40-CD3zeta
(SEQ ID NO: 106)
KRGRKKLLYIFKQPFMRPVQTTQEEDGCSCRFPEEEEGGCELRRDQRLP
PDAHKPPGGGSFRTPIQEEQADAHSTLAKIRVKFSRSADAPAYQQGQNQ
LYNELNLGRREEYDVLDKRRGRDPEMGGKPRRKNPQEGLYNELQKDKMA
EAYSEIGMKGERRRGKGHDGLYQGLSTATKDTYDALHMQALPPR

The cytoplasmic domain of the CAR can be designed to comprise the CD3-zeta signal transduction domain by itself or combined with any other desired cytoplasmic domain(s) useful in the context of the CAR of the invention, such as a 4-1BB domain, a CD28 domain, and/or an OX40 domain. For example, the cytoplasmic domain of the CAR can comprise a CD3 zeta chain portion and a costimulatory signaling region. The costimulatory signaling region refers to a portion of the CAR comprising the intracellular domain of a costimulatory molecule. Thus, while the invention is exemplified primarily with 4-1BB, CD28, and OX40 as the costimulatory or signaling element(s), other additional costimulatory or signaling elements are within the scope of the invention. Exemplary sequences of costimulatory and intracellular domains are provided herein. Other exemplary 4-1BB costimulatory domains are described in US Patent Publication 20050113564, which is incorporated by reference herein.

In some embodiments, any of the CARs provided herein comprise a CD3zeta signaling domain. In some embodiments, the CD3zeta domain is derived from a human. In some embodiments, the CD3zeta domain comprises an amino acid sequence that is at least 70%, 75%, 80%, 85%, 90%, 95%, 96%, 97%, 98%, or 99% identical to the amino acid sequence of SEQ ID NO: 107. In some embodiments, the CD3zeta domain comprises the amino acid sequence of SEQ ID NO: 107.

In some embodiments, any of the CARs provided herein comprise one or more (e.g., 2, 3, 4, or 5) costimulatory domains. In some embodiments, any of the CARs provided herein comprise a CD28, a 41BB, and/or an OX40 costimulatory domain. In some embodiments, the costimulatory domain is derived from a human. In some embodiments, the CAR comprises one or more of the amino acid sequences of any of SEQ ID NOs: 100-102, or any variants thereof that are at least 70%, 75%, 80%, 85%, 90%, 95%, 96%, 97%, 98%, or 99% identical thereto. It should be appreciated that the cytoplasmic domain of the car may comprise any combination of signaling and/or cytoplasmic domains. For example, the cytoplasmic domain may comprise any of the following exemplary combination of domains: CD28-CD3zeta; 41BB-CD3zeta; OX40-CD3zeta; or 41BB-OX40-CD3zeta. In some embodiments, the cytoplasmic domain of the CAR comprises the amino acid sequence of any of SEQ ID NOs: 103-106, or any variants thereof that are at least 70%, 75%, 80%, 85%, 90%, 95%, 96%, 97%, 98%, or 99% identical thereto.

### Exemplary Full-Length CARs

As provided herein, the disclosure contemplates CARx comprising an anti-BCMA binding domain (e.g., human or humanized BCMA binding domain as described herein), a transmembrane domain, and a cytoplasmic domain. It should be appreciated that any of the CARs provided herein can be produced using any combination of domains or peptides described herein, for example the CAR can comprise any anti-BCMA binding domain, transmembrane, cytoplasmic domain, signal peptide, or hinge region provided herein. Exemplary CARs and variants thereof are provided below.

CAR-R1
(SEQ ID NO: 19)
MALPVTALLLPLALLLHAARPDIVLTQSPPSLAMSLGKRATISCRASESVTILGSHLIH
WYQQKPGQPPTLLIQLASNVQTGVPARFSGSGSRTDFTLTIDPVEEDDVAVYYCLQSR
TIPRTFGGGTKLEIKGSTSGSGKPGSGEGSTKGQIQLVQSGPELKKPGETVKISCKASG
YTFTDYSINWVKRAPGKGLKWMGWINTTETREPAYAYDFRGRFAFSLETSASTAYLQI
NNLKYEDTATYFCALDYSYAMDYWGQGTSVTVSSFVPVFLPAKPTTTPAPRPPTPAPT
IASQPLSLRPEACRPAAGGAVHTRGLDFACDIYIWAPLAGTCGVLLLSLVITLYCNHRN
RSKRSRLLHSDYMNMTPRRPGPTRKHYQPYAPPRDFAAYRSRVKFSRSADAPAYQQG
QNQLYNELNLGRREEYDVLDKRRGRDPEMGGKPRRKNPQEGLYNELQKDKMAEAY
SEIGMKGERRRGKGHDGLYQGLSTATKDTYDALHMQALPPR
CAR-K′
(SEQ ID NO: 20)
MALPVTALLLPLALLLHAARPDIVLTQSPASLAVSPGQRATITCRASESVSFLGINLIHW
YQQKPGQPPKLLIYSASNLQSGVPARFSGSGSGTDFTLTISSVEPEDTANYYCLQSRTL
PRTFGQGTKVEIKGSTSGSGKPGSGEGSTKGQIQLVQSGPELKKPGGSVKISCKASGY
TFTSYSINWVRQAPGKGLEWVGWINTETREPAYAQGFTGRFTFSADTSKSMAYLQIN
SLRAEDTAVYYCALDYLYSLDFWGQGTLVTVSSFVPVFLPAKPTTTPAPRPPTPAPTIA
SQPLSLRPEACRPAAGGAVHTRGLDFACDIYIWAPLAGTCGVLLLSLVITLYCNHRNR
VKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
CAR-R′
(SEQ ID NO: 21)
MALPVTALLLPLALLLHAARPDIVLTQSPASLAVSPGQRATITCRASESVSFLGINLIHW
YQQKPGQPPKLLIYSASNLQSGVPARFSGSGSGTDFTLTISSVEPEDTANYYCLQSRTL
PRTFGQGTKVEIKGSTSGSGKPGSGEGSTKGQIQLVQSGPELKKPGGSVKISCKASGY
TFTSYSINWVRQAPGKGLEWVGWINTETREPAYAQGFTGRFTFSADTSKSMAYLQIN
SLRAEDTAVYYCALDYLYSLDFWGQGTLVTVSSFVPVFLPAKPTTTPAPRPPTPAPTIA
SQPLSLRPEACRPAAGGAVHTRGLDFACDIYIWAPLAGTCGVLLLSLVITLYCNHRNK
RGRKKLLYIFKQPFMRPVQTTQEEDGCSCRFPEEEEGGCELRVKFSRSADAPAYQQG
QNQLYNELNLGRREEYDVLDKRRGRDPEMGGKPRRKNPQEGLYNELQKDKMAEAY
SEIGMKGERRRGKGHDGLYQGLSTATKDTYDALHMQALPPR
CAR-J′
(SEQ ID NO: 22)
MALPVTALLLPLALLLHAARPDIVLTQSPASLAVSPGQRATITCRASESVSFLGINLIHW
YQQKPGQPPKLLIYSASNLQSGVPARFSGSGSGTDFTLTISSVEPEDTANYYCLQSKNF
PRTFGQGTKVEIKGSTSGSGKPGSGEGSTKGQIQLVQSGPELKKPGGSVKISCKASGY
TFTSYSINWVRQAPGKGLEWVGWINTETREPAYAQGFTGRFTFSADTSKSMAYLQIN
SLRAEDTAVYYCALDYLYSLDFWGQGTLVTVSSFVPVFLPAKPTTTPAPRPPTPAPTIA
SQPLSLRPEACRPAAGGAVHTRGLDFACDIYIWAPLAGTCGVLLLSLVITLYCNHRNR
VKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
CAR-L′
(SEQ ID NO: 23)
MALPVTALLLPLALLLHAARPDIVLTQSPASLAVSPGQRATITCRASESVSFLGINLIHW
YQQKPGQPPKLLIYSASNLQSGVPARFSGSGSGTDFTLTISSVEPEDTANYYCLQSKNF
PRTFGQGTKVEIKGSTSGSGKPGSGEGSTKGQIQLVQSGPELKKPGGSVKISCKASGY
TFTSYSINWVRQAPGKGLEWVGWINTETREPAYAQGFTGRFTFSADTSKSMAYLQIN
SLRAEDTAVYYCALDYLYSLDFWGQGTLVTVSSFVPVFLPAKPTTTPAPRPPTPAPTIA
SQPLSLRPEACRPAAGGAVHTRGLDFACDIYIWAPLAGTCGVLLLSLVITLYCNHRNK
RGRKKLLYIFKQPFMRPVQTTQEEDGCSCRFPEEEEGGCELRVKFSRSADAPAYQQG
QNQLYNELNLGRREEYDVLDKRRGRDPEMGGKPRRKNPQEGLYNELQKDKMAEAY
SEIGMKGERRRGKGHDGLYQGLSTATKDTYDALHMQALPPR
CAR-O′
(SEQ ID NO: 24)
MALPVTALLLPLALLLHAARPDIVLTQSPASLAVSPGQRATITCRASESVTILGSHLIHW
YQQKPGQPPKLLINLASNVNTGVPARFSGSGSGTDFTLTISSVEPEDTANYYCLQSRTL
PRTFGQGTKVEIKGSTSGSGKPGSGEGSTKGQIQLVQSGPELKKPGGSVKISCKASGY
TFTDYSINWVRQAPGKGLEWVGWINTTETREPAYAYDFTGRFTFSADTSKSMAYLQIN
SLRAEDTAVYYCALDYTYGMDYWGQGTLVTVSSFVPVFLPAKPTTTPAPRPPTPAPTI
ASQPLSLRPEACRPAAGGAVHTRGLDFACDIYIWAPLAGTCGVLLLSLVITLYCNHRN
RVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
CAR-S′
(SEQ ID NO: 25)
MALPVTALLLPLALLLHAARPDIVLTQSPASLAVSPGQRATITCRASESVTILGSHLIHW
YQQKPGQPPKLLINLASNVNTGVPARFSGSGSGTDFTLTISSVEPEDTANYYCLQSRTL
PRTFGQGTKVEIKGSTSGSGKPGSGEGSTKGQIQLVQSGPELKKPGGSVKISCKASGY
TFTDYSINWVRQAPGKGLEWVGWINTTETREPAYAYDFTGRFTFSADTSKSMAYLQIN
SLRAEDTAVYYCALDYTYGMDYWGQGTLVTVSSFVPVFLPAKPTTTPAPRPPTPAPTI
ASQPLSLRPEACRPAAGGAVHTRGLDFACDIYIWAPLAGTCGVLLLSLVITLYCNHRN
KRGRKKLLYIFKQPFMRPVQTTQEEDGCSCRFPEEEEGGCELRVKFSRSADAPAYQQ
GQNQLYNELNLGRREEYDVLDKRRGRDPEMGGKPRRKNPQEGLYNELQKDKMAEA
YSEIGMKGERRRGKGHDGLYQGLSTATKDTYDALHMQALPPR
CAR-II
(SEQ ID NO: 26)
MALPVTALLLPLALLLHAARPDIVLTQSPSSLSASVGDRATISCRASESVTILGSHLIHW
YQQKPGQAPKLLIQLASNVQTGVPARFSGSGSGTDFTLTISSVEPEDVAVYYCLQSRTI
PRTFGQGTKVEIKGSTSGSGKPGSGEGSTKGQIQLVQSGPELKKPGGSVKISCAASGY
TFTDYSINWVRQAPGKGLEWVGWINTTETREPAYAYDFRGRFTFSADTSKSTAYLQMN
SLRAEDTAVYYCALDYSYAMDYWGQGTLVTVSSFVPVFLPAKPTTTPAPRPPTPAPTI
ASQPLSLRPEACRPAAGGAVHTRGLDFACDIYIWAPLAGTCGVLLLSLVITLYCNHRN
RVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
CAR-10dU
(SEQ ID NO: 27)
MALPVTALLLPLALLLHAARPDIVLTQSPSSLSASVGDRATISCRASESVTILGSHLIHW
YQQKPGQAPKLLIQLASNVQTGVPARFSGSGSGTDFTLTISSVEPEDVAVYYCLQSRTI
PRTFGQGTKVEIKGSTSGSGKPGSGEGSTKGQIQLVQSGPELKKPGGSVKISCAASGY
TFTDYSINWVRQAPGKGLEWVGWINTTETREPAYAYDFRGRFTFSADTSKSTAYLQMN
SLRAEDTAVYYCALDYSYAMDYWGQGTLVTVSSFVPVFLPAKPTTTPAPRPPTPAPTI
ASQPLSLRPEACRPAAGGAVHTRGLDFACDIYIWAPLAGTCGVLLLSLVITLYCNHRN
RVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
CAR-17′
(SEQ ID NO: 28)
MALPVTALLLPLALLLHAARPDIVLTQSPASLAVSPGQRATITCRASESVTILGSHLIHW
YQQKPGQPPKLLIQLASNVQTGVPARFSGSGSGTDFTLTISSVEPEDTANYYCLQSRTI
PRTFGQGTKVEIKGSTSGSGKPGSGEGSTKGQIQLVQSGPELKKPGGSVKISCKASGY
TFTDYSINWVRQAPGKGLEWVGWINTETREPAYAYDFTGRFTFSADTSKSMAYLQIN
SLRAEDTAVYYCALDYSYAMDYWGQGTLVTVSSFVPVFLPAKPTTTPAPRPPTPAPTI
ASQPLSLRPEACRPAAGGAVHTRGLDFACDIYIWAPLAGTCGVLLLSLVITLYCNHRN
RVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR

In some embodiments, the full-length amino acid sequence of the CAR is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any one of SEQ ID NOs: 19-28, or to any of the CARs provided herein. In some embodiments, the CAR comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to any one of the amino acid sequences set forth in SEQ ID NOS: 19-28, or any of the fusion proteins provided herein. In some embodiments, the CAR comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 200, at least 300, at least 400, or at least 500 identical contiguous amino acid residues as compared to any one of the amino acid sequences set forth in SEQ ID NOs: 19-28, or any of the fusion proteins provided herein.

### Nucleic Acids and Vectors

In some embodiments, the present invention encompasses a nucleic acid molecule (e.g., DNA or RNA) that encodes a CAR (e.g., any of the CARs provided herein). In some embodiments, the nucleic acid molecule is a DNA. In some embodiments, the nucleic acid molecule is an RNA. In some embodiments, the nucleic acid molecule comprises the sequence of a CAR, wherein the sequence comprises a nucleic acid sequence that encodes an antigen binding domain (e.g., an anti-BCMA binding domain) operably linked to the nucleic acid sequence encoding one or more of an extracellular domain, a transmembrane domain, and an intracellular domain. In some embodiments, the nucleic acid molecule encodes a CAR having an arrangement selected from one of the following exemplary, non-limiting arrangements:
[antigen binding domain]-[transmembrane domain]-[cytoplasmic domain];
[antigen binding domain]-[hinge region]-[transmembrane domain]-[cytoplasmic domain];
[Signal peptide]-[antigen binding domain]-[transmembrane domain]-[cytoplasmic domain]; or
[Signal peptide]-[antigen binding domain]-[hinge region]-[transmembrane domain]-[cytoplasmic domain];

In some embodiments, the nucleic acid sequence of the CAR comprises a cytoplasmic domain having an arrangement selected from one of the following exemplary, non-limiting arrangements:
[CD3zeta];
[CD28]-[CD3zeta];
[41BB]-[CD3zeta];
[OX40]-[CD3zeta]; or
[41BB]-[OX40]-[CD3zeta]

In some embodiments, the above exemplary, non-limiting arrangements are from left to right, N-terminus to C-terminus of the CAR. In some embodiments, each instance of “]-[” indicates the presence of an optional spacer sequence.

It should be appreciated that any of the nucleic acid molecules provided herein may encode, or include other features, for example a 5′ untranslated region (5′ UTR), a 3′ untranslated region (3′ UTR), a poly-adenine tail (polyA), a 7-methylguanosine cap (m7G), an internal ribosome entry site (IRES) and/or an open reading frame. In some embodiments, the disclosure provides an RNA having the following arrangement of features, or a DNA that encodes the same:
5′-[5′ UTR]-[CAR]-3′
5′-[m⁷G cap]-[5′ UTR]-[CAR]-3′
5′-[m⁷G cap]-[5′ UTR]-[CAR]-[polyA]-3′
5′-[CAR]-[3′ UTR]-[polyA]-3′
5′-[5′ UTR]-[CAR]-[3′ UTR]-3′
5′-[5′ UTR]-[CAR]-[3′ UTR]-[polyA]-3′
5′-[m⁷G cap]-[5′ UTR]-[CAR]-[3′ UTR]-[PolyA]-3′

In some embodiments, the nucleic acids provided include chemical structures with the ability to promote stability and/or translation efficiency. In some embodiments, the RNA preferably has 5′ and 3′ UTRs. In one embodiment, the 5′ UTR is between one and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

In some embodiments, the 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be 5′ UTR of an RNA virus whose RNA genome is stable in cells. In other embodiments various nucleotide analogues can be used in the 3′ or 5′ UTR to impede exonuclease degradation of the mRNA.

In some embodiments, the disclosure provides 5′ and/or 3′ UTRs that are particularly useful for expressing CARs in cells (e.g., a T cell). In some embodiments, the 5′ UTR is a 5′ UTR from Yi1, IgG, CD8, Myo, CD3, AFP, Actin, IFNG, ACTBL2, a synthetic sequence, or a variant thereof. In some embodiments, the 5′ UTR is from a human or mouse. Exemplary 5′ UTR sequences are provided below. In some embodiments, the 5′ UTR comprises a nucleic acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the nucleic acid sequences set forth in any one of SEQ ID NOs: 73-82. In some embodiments, the 5′ UTR comprises a nucleic acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, or more mutations compared to any one of the nucleic acid sequences set forth in SEQ ID NOs: 71-82. In some embodiments, the 5′ UTR comprises a nucleic acid sequence that has at least 5, at least 10, at least 15, at least 20, at least 25, at least 30, at least 35, at least 40, at least 45, or at least 50 identical contiguous nucleotides as compared to any one of the nucleic acid sequences set forth in SEQ ID NOs: 71-82.

5′ UTR - Yi1
(SEQ ID NO: 73)
GGGAGACCCAAGCTGGCTAGCCTCGCAGTTCGGCGGTCCCGCGGGTCTGT
CTGTTGCTTCAACAGTGTTTGGACGGAACAGATCCGGGGACTCTCTTCCA
GCC
5′ UTR - IgG
(SEQ ID NO: 74)
AGACCCAAGCTGGCTAGCTCTAAAGAAGCCCCTGGGAGCACAGCTCATCA
CC
5′ UTR - CD8
(SEQ ID NO: 75)
AGACCCAAGCTGGCTAGCAGCTCCTCACCCACCCCAGCCGCGACTGTCTC
CGCCGAGCCCCCGGGGCCAGGTGTCCCGGGCGCGCCCCG
5′ UTR - Myo
(SEQ ID NO: 76)
GTGGAACACTTCTGAACCTGCATTTTTATCTGGAACTCCAGAAGCAGAAT
CCTTTGCTAAATAAATCGCAGCC
5′ UTR - CD3
(SEQ ID NO: 77)
AGAGAAGCAGACATCTTCTAGTTCCTCCCCCACTCTCCTCTTTCCGGTAC
CTGTGAGTCAGCTAGGGGAGGGCAGCTCTCACCCAGGCTGATAGTTCGGT
GACCTGGCTTTATCTACTGGATGAGTTCCGCTGGGAG
5′ UTR - AFP
(SEQ ID NO: 78)
AGACCCAAGCTGGCTAGCATATTGTGCTTCCACCACTGCCAATAACAAAA
TAACTAGCAACC
5′ UTR - R1 synthetic sequence
(SEQ ID NO: 79)
AAATAAGAGAGAAAAGAAGAGTAAGAAGAAATATAAGAGCCACC
5′ UTR - Actin
(SEQ ID NO: 80)
CGCGTCCGCCCCGCGAGCACAGAGCCTCGCCTTTGCCGATCCGCCGCCCG
TCCACACCCGCCGCCAGCTCACC
5′ UTR - IFNG
(SEQ ID NO: 81)
CACATTGTTCTGATCATCTGAAGATCAGCTATTAGAAGAGAAAGATCAGT
TAAGTCCTTTGGACCTGATCAGCTTGATACAAGAACTACTGATTTCAACT
TCTTTGGCTTAATTCTCTCGGAAACG
5′ UTR - ACTBL2
(SEQ ID NO: 82)
GTGTCTGAAAGCATTTCTGGAGTGTTTTAGGCCTGTTCACTTTCTCTTAC
TCACTGTCTATTCACTTGTCCTGTTCACTCGTCTGGAAGATCTCAGCCAG
CACC

In some embodiments, the disclosure provides 3′ UTRs that are particularly useful for expressing CARs in cells (e.g., a T cell). In some embodiments. the 3′ UTR is a 3′ UTR from human beta globin, IgG, Actin, AFP, CD3, IFNG, Myo, FoxP3, GAPDH, GATA3, H2AFV, RORC, SOC2, mouse alpha globin, GATA3, or a variant thereof. In some embodiments, the 3′ UTR is from a human or mouse. Exemplary 3′ UTR sequences are provided below. In some embodiments, the 3′ UTR comprises a nucleic acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the nucleic acid sequences set forth in any one of SEQ ID NOs: 83-99. In some embodiments, the 3′ UTR comprises a nucleic acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, or more mutations compared to any one of the nucleic acid sequences set forth in SEQ ID NOs: 83-99. In some embodiments, the 3′ UTR comprises a nucleic acid sequence that has at least 5, at least 10, at least 15, at least 20, at least 25, at least 30, at least 35, at least 40, at least 45, or at least 50 identical contiguous nucleotides as compared to any one of the nucleic acid sequences set forth in SEQ ID NOs: 83-99.

3′ UTR - Human beta globin
(SEQ ID NO: 83)
GGGCCCGTTTAAACCCGCTGGCTCGCTTTCTTGCTGTCCAATTTCTATTAAAGGTTC
CTTTGTTCCGTAAGTCCAACTACTAAACTGGGGGATATTATGAAGGGCCTTGAGCA
TCTGGATTCTGCCTAATAAAAAACATTTATTTTCATTGC
3′ UTR - IgG
(SEQ ID NO: 84)
TGAGTGCGACGGCCGGCAAGCCCCCGCTCCCCGGGCTCTCGCGGTCGCACGAGG
ATGCTTGGCACGTACCCCGTGTACATACTTCCCGGGCGCCCAGCATGGAAATAAAG
CACCCAGCGCTGCCCTGGGCCCCTGCG
3′ UTR - Actin
(SEQ ID NO: 85)
GCGGACTATGACTTAGTTGCGTTACACCCTTTCTTGACAAAACCTAACTTGCGCAG
AAAACAAGATGAGATTGGCATGGCTTTATTTGTTTTTTTTGTTTTGTTTTGGTTTTT
TTTTTTTTTTGGCTTGACTCAGGATTTAAAAACTGGAACGGTGAAGGTGACAGCA
GTCGGTTGGAGCGAGCATCCCCCAAAGTTCTCAATGTGGCCGAGGACTTTGATTG
CACATTGTTGTTTTTTTAATAGTCATTCCAAATATGAGATGCGTTGTTACAGGAAGT
CCCTTGCCATCCTAAAAGCCACCCCACTTCTCTCTAAGGAGAATGGCCCAGTCCTC
TCCCAAGTCCACACAGGGGAGGTGATAGCATTGCTTTCGTGTAAATTATGTAATGC
AAAATTTTTTTAATCTTCGCCTTAATACTTTTTTATTTTGTTTTATTTTGAATGATGAG
CCTTCGTGCCCCCCCTTCCCCCTTTTTTGTCCCCCAACTTGAGATGTATGAAGGCTT
TTGGTCTCCCTGGGAGTGGGTGGAGGCAGCCAGGGCTTACCTGTACACTGACTTG
AGACCAGTTGAATAAAAGTGCACACCTT
3′ UTR - AFP
(SEQ ID NO: 86)
ATTACTTCAGGGGAAGAGATGACAAAACGAGTCTTTCATTCGGTGTGAACTTTTCT
CTTTAATTTTAACTGATTTAACACTTTTTGTGAATTAATGAAATGATAAAGACTTTTA
TGTGAGATTTCCTTATCACAGAAATAAAATATCTCCAAATGTTTCCTTTTC
3′ UTR - CD3
(SEQ ID NO: 87)
ACCTGAGACTGGTGGCTTCTAGAAGCAGCCATTACCAACTGTACCTTCCCTTCTTG
CTCAGCCAATAAATATATCCTCTTTCACTCAG
3′ UTR - IFNG
(SEQ ID NO: 88)
TGGTTGTCCTGCCTGCAATATTTGAATTTTAAATCTAAATCTATTTATTAATATTTAAC
ATTATTTATATGGGGAATATATTTTTAGACTCATCAATCAAATAAGTATTTATAATAGC
AACTTTTGTGTAATGAAAATGAATATCTATTAATATATGTATTATTTATAATTCCTATAT
CCTGTGACTGTCTCACTTAATCCTTTGTTTTCTGACTAATTAGGCAAGGCTATGTGA
TTACAAGGCTTTATCTCAGGGGCCAACTAGGCAGCCAACCTAAGCAAGATCCCAT
GGGTTGTGTGTTTATTTCACTTGATGATACAATGAACACTTATAAGTGAAGTGATAC
TATCCAGTTACTGCCGGTTTGAAAATATGCCTGCAATCTGAGCCAGTGCTTTAATG
GCATGTCAGACAGAACTTGAATGTGTCAGGTGACCCTGATGAAAACATAGCATCT
CAGGAGATTTCATGCCTGGTGCTTCCAAATATTGTTGACAACTGTGACTGTACCCA
AATGGAAAGTAACTCATTTGTTAAAATTATCAATATCTAATATATATGAATAAAGTGT
AAGTTCACAAC
3′ UTR - Myo
(SEQ ID NO: 89)
ACACACCTGCCTGATGCTATCAAGAGGCTGAAGAAAGCGCCAAATGTGCTATTTTT
TGGTCACTTGCTTTATGACGTTTATTTTCCTGTTAAAGCTGAATAAATAAAAACTAC
AGTAAATGTA
3′ UTR - FoxP3
(SEQ ID NO: 90)
CCTCAAGATCAAGGAAAGGAGGATGGACGAACAGGGGCCAAACTGGTGGGAGG
CAGAGGTGGTGGGGGCAGGGATGATAGGCCCTGGATGTGCCCACAGGGACCAAG
AAGTGAGGTTTCCACTGTCTTGCCTGCCAGGGCCCCTGTTCCCCCGCTGGCAGCC
ACCCCCTCCCCCATCATATCCTTTGCCCCAAGGCTGCTCAGAGGGGCCCCGGTCCT
GGCCCCAGCCCCCACCTCCGCCCCAGACACACCCCCCAGTCGAGCCCTGCAGCC
AAACAGAGCCTTCACAACCAGCCACACAGAGCCTGCCTCAGCTGCTCGCACAGA
TTACTTCAGGGCTGGAAAAGTCACACAGACACACAAAATGTCACAATCCTGTCCC
TCACTCAACACAAACCCCAAAACACAGAGAGCCTGCCTCAGTACACTCAAACA
ACCTCAAAGCTGCATCATCACACAATCACACACAAGCACAGCCCTGACAACCCACA
CACCCCAAGGCACGCACCCACAGCCAGCCTCAGGGCCCACAGGGGCACTGTCAA
CACAGGGGTGTGCCCAGAGGCCTACACAGAAGCAGCGTCAGTACCCTCAGGATC
TGAGGTCCCAACACGTGCTCGCTCACACACACGGCCTGTTAGAATTCACCTGTGT
ATCTCACGCATATGCACACGCACAGCCCCCCAGTGGGTCTCTTGAGTCCCGTGCA
GACACACACAGCCACACACACTGCCTTGCCAAAAATACCCCGTGTCTCCCCTGCC
ACTCACCTCACTCCCATTCCCTGAGCCCTGATCCATGCCTCAGCTTAGACTGCAGA
GGAACTACTCATTTATTTGGGATCCAAGGCCCCCAACCCACAGTACCGTCCCCAAT
AAACTGCAGCCGAGCTCCCCAC
3′ UTR - GAPDH
(SEQ ID NO: 91)
GAAGTCTGTTCCTGTCCTCCCTGTGCAGGGTATCCTGTAGGGTGACCTGGAATTCG
AATTCTGTTTCCCTTGTAAAATATTTGTCTGTCTCTTTTTT
3′ UTR - GATA3 (ORF sequence)
(SEQ ID NO: 92)
CTATGAAGAAGGAAGGCATCCAGACCAGAAACCGAAAAATGTCTAGCAAATCCA
AAAAGTGCAAAAAAGTGCATGACTCACTGGAGGACTTCCCCAAGAACAGCTCGT
TTAACCCGGCCGCCCTCTCCAGACACATGTCCTCCCTGAGCCACATCTCGCCCTTC
AGCCACTCCAGCCACATGCTGACCACGCCCACGCCGATGCACCCGCCATCCAGC
3′ UTR - H2AFV
(SEQ ID NO: 93)
AGGGATGCTTTAACCAACCCTCTTCCTCCCCGTCATTGTACTGTAACTGGGACAGA
AGAAATAATGGGGATATGTGGAATTTTTAACAACAGTTAAATGGAAAAGCATAGAC
AATTACTGTAGACATGATAAAAGAAACATTTGTATGTTCTTAGACTCGAAGTTTGAT
AAAAGTACCTTTTCATGTGGTGACAGTTGTGTGTTGATTGGCTAGGTTTCTCCCGT
GTGTTTTATACAAAAATGGAATTGATAAACCATTTTTTACAAAATTAATTTGTCTCA
AAACTGTTCTGTTCATGATGTATTAGAAATATTTTACTCAGACTTTAAATATTTTAAA
TCTCAGATTGGTTATTCAGAGTAACCTTAGAACAGAAATTGGGAATATATCTTTACA
ATGATTGATACCATGGTATATTGACTCTTAGATGCTATTGATCTGTAGCACCATTTTT
TACAAACGACTAAGGAAAAAACCTGCCAATTAAATCATGATATGCCATCAATTATG
AGACATCCCAATTTGAGAGATGTTAGATTATAGAAAAGTATGCATTTATGACTGAA
ATGGTAGTGGAATTATTTGAATTCTACACCAAGCACTTACCATGTGCCAGGCCCTTT
GCAGAGTGCTCTACTGACCAAGAAAGTTGTTGCTGCCACATTATAGATGTGGAGC
CTAAGGGTCACAGAAATTGTGTGCTATGCCAAAAAACATTGAACTGGTAGATAGA
AAATGACAGAGCTAGGATTCAAACCTAGATCTGGCTGACTCCAGAGCCTAGTTTTA
CCTGGAATTGATGTTCAGTTTATCAAAGGTTTCTCCTTTTGGTTTAAAATCCCAATT
TTTGGCCTGGCATTGTGGTTTACGCCTGTAATCCCAACAC
3′ UTR - RORC
(SEQ ID NO: 94)
CCTGGAAGAGGGACTCCTTGCCTCTCCCTATGGCCTGCTGGCCCACCTCCCTGGA
CCCCGTTCCACCCTCACCCTTTTCCTTTCCCATGAACCCTGGAGGGTGGTCCCCAC
CAGCTCTTTGGAAGTGAGCAGATGCTGCGGCTGGCTTTCTGTCAGCAGGCCGGCC
TGGCAGTGGGACAATCGCCAGAGGGTGGGGCTGGCAGAACACCATCTCCAGCCT
CAGCTTTGACCTGTCTCATTTCCCATATTCCTTCACACCCAGCTTCTGGAAGGCATG
GGGTGGCTGGGATTTAAGGACTTCTGGGGGACCAAGACATCCTCAAGAAAACAG
GGGCATCCAGGGCTCCCTGGATGAATAGAATGCAATTCATTCAGAAGCTCAGAAG
CTAAGAATAAGCCTTTGAAATACCTCATTGCATTTCCCTTTGGGCTTCGGCTTGGG
GAGATGGATCAAGCTCAGAGACTGGCAGTGAGAGCCCAGAAGGACCTGTATAAA
ATGAATCTGGAGCTTTACATTTTCTGCCTCTGCCTTCCTCCCAGCTCAGCAAGGAA
GTATTTGGGCACCCTACCCTTTACCT
3′ UTR - SOD2
(SEQ ID NO: 95)
ACCACGATCGTTATGCTGATCATACCCTAATGATCCCAGCAAGATAATGTCCTGTCC
TCTAAGATGTGCATCAAGCCTGGTACATACTGAAAACCCTATAAGGTCCTGGATAA
TTTTTGTTTGATTATTCATTGAAGAAACATTTATTTTCCAATTGTGTGAAGTTTTTGA
CTGTTAATAAAAGAATCTGTCAACCATCAAAGAGGTCTGCATTATGCTTGCATGTC
AAAAACTTTAAAAATCCTATAATCTTC
3′ UTR - mouse alpha globulin
(SEQ ID NO: 96)
GCGGCCGCTTAATTAAGCTGCCTTCTGCGGGGCTTGCCTTCTGGCCATGCCCTTCT
TCTCTCCCTTGCACCTGTACCTCTTGGTCTTTGAATAAAGCCTGAGTAGGAAGTCT
AG
3′ UTR - GATA3 (full)
(SEQ ID NO: 97)
GCCCTGCTCGATGCTCACAGGGCCCCCAGCGAGAGTCCCTGCAGTCCCTTTCGAC
TTGCATTTTTGCAGGAGCAGTATCATGAAGCCTAAACGCGATGGATATATGTTTTTG
AAGGCAGAAAGCAAAATTATGTTTGCCACTTTGCAAAGGAGCTCACTGTGGTGTC
TGTGTTCCAACCACTGAATCTGGACCCCATCTGTGAATAAGCCATTCTGACTCATAT
CCCCTATTTAACAGGGTCTCTAGTGCTGTGAAAAAAAAAATGCTGAACATTGCATA
TAACTTATATTGTAAGAAATACTGTACAATGACTTTATTGCATCTGGGTAGCTGTAA
GGCATGAAGGATGCCAAGAAGTTTAAGGAATATGGGAGAAATAGTGTGGAAATTA
AGAAGAAACTAGGTCTGATATTCAAATGGACAAACTGCCAGTTTTGTTTCCTTTCA
CTGGCCACAGTTGTTTGATGCATTAAAAGAAAATAAAAAAAAGAAAAAAGAGAA
AAGAAAAAAAAAGAAAAAAGTTGTAGGCGAATCATTTGTTCAAAGCTGTTGGCC
TCTGCAAAGGAAATACCAGTTCTGGGCAATCAGTGTTACCGTTCACCAGTTGCCG
TTGAGGGTTTCAGAGAGCCTTTTTCTAGGCCTACATGCTTTGTGAACAAGTCCCTG
TAATTGTTGTTTGTATGTATAATTCAAAGCACCAAAATAAGAAAAGATGTAGATTTA
TTTCATCATATTATACAGACCGAACTGTTGTATAAATTTATTTACTGCTAGTCTTAAG
AACTGCTTTCTTTCGTTTGTTTGTTTCAATATTTTCCTTCTCTCTCAATTTTTGGTTG
AATAAACTAGATTACATTCAGTTGGCCTAAGGTGGTTGTGCTCGGAGGGTTTCTTG
TTTCTTTTCCATTTTGTTTTTGGATGATATTTATTAAATAGCTTCTAAGAGTCCGGCG
GCATCTGTCTTGTCCCTATTCCTGCAGCCTGTGCTGAGGGTAGCAGTGTATGAGCT
ACCAGCGTGCATGTCAGCGACCCTGGCCCGACAGGCCACGTCCTGCAATCGGCCC
GGCTGCCTCTTCGCCCTGTCGTGTTCTGTGTTAGTGATCACTGCCTTTAATACAGTC
TGTTGGAATAATATTATAAGCATAATAATAAAGTGAAAATATTTTAAAACTACAA
3′ UTR - GATA3 (first half)
(SEQ ID NO: 98)
GCCCTGCTCGATGCTCACAGGGCCCCCAGCGAGAGTCCCTGCAGTCCCTTTCGAC
TTGCATTTTTGCAGGAGCAGTATCATGAAGCCTAAACGCGATGGATATATGTTTTTG
AAGGCAGAAAGCAAAATTATGTTTGCCACTTTGCAAAGGAGCTCACTGTGGTGTC
TGTGTTCCAACCACTGAATCTGGACCCCATCTGTGAATAAGCCATTCTGACTCATAT
CCCCTATTTAACAGGGTCTCTAGTGCTGTGAAAAAAAAAATGCTGAACATTGCATA
TAACTTATATTGTAAGAAATACTGTACAATGACTTTATTGCATCTGGGTAGCTGTAA
GGCATGAAGGATGCCAAGAAGTTTAAGGAATATGGGAGAAATAGTGTGGAAATTA
AGAAGAAACTAGGTCTGATATTCAAATGGACAAACTGCCAGTTTTGTTTCCTTTCA
CTGGCCACAGTTGTTTGATGCATTAAAAGAAAATAAAAAAAAGAAAAAAGAGAA
AAGAAAAAAAAAGAAAAAA
3′ UTR - GATA3 (second half)
(SEQ ID NO: 99)
GTTGTAGGCGAATCATTTGTTCAAAGCTGTTGGCCTCTGCAAAGGAAATACCAGTT
CTGGGCAATCAGTGTTACCGTTCACCAGTTGCCGTTGAGGGTTTCAGAGAGCCTT
TTTCTAGGCCTACATGCTTTGTGAACAAGTCCCTGTAATTGTTGTTTGTATGTATAAT
TCAAAGCACCAAAATAAGAAAAGATGTAGATTTATTTCATCATATTATACAGACCG
AACTGTTGTATAAATTTATTTACTGCTAGTCTTAAGAACTGCTTTCTTTCGTTTGTTT
GTTTCAATATTTTCCTTCTCTCTCAATTTTTGGTTGAATAAACTAGATTACATTCAGT
TGGCCTAAGGTGGTTGTGCTCGGAGGGTTTCTTGTTTCTTTTCCATTTTGTTTTTGG
ATGATATTTATTAAATAGCTTCTAAGAGTCCGGCGGCATCTGTCTTGTCCCTATTCCT
GCAGCCTGTGCTGAGGGTAGCAGTGTATGAGCTACCAGCGTGCATGTCAGCGACC
CTGGCCCGACAGGCCACGTCCTGCAATCGGCCCGGCTGCCTCTTCGCCCTGTCGT
GTTCTGTGTTAGTGATCACTGCCTTTAATACAGTCTGTTGGAATAATATTATAAGCAT
AATAATAAAGTGAAAATATTTTAAAACTACAA

In some embodiments, the disclosure provides nucleic acid sequences, each of which encodes one or more of the CARs provided herein. Exemplary nucleic sequences that encode such CARs are provided in SEQ ID NOs: 29-47. However, it should be appreciated that variants of the sequences provided herein are also contemplated. In some embodiments, the CAR is encoded by a nucleic acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the nucleic acid sequences set forth in any one of SEQ ID NOs: 29-47. In some embodiments, the CAR is encoded by a nucleic acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, or more mutations (e.g., at least 50, 100, 200, 300, 400, 500, or 600) compared to any one of the nucleic acid sequences set forth in SEQ ID NOs: 29-47. In some embodiments, the nucleic acid sequence encoding the CAR comprises a nucleic acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 200, at least 300, at least 400, at least 500, at least 600, at least 700, at least 800, at least 900, at least 1000, at least, 1100, at least 1200, at least 1300, at least 1400, at least 1500, at least 1600, or at least 1700 identical contiguous nucleotides as compared to any one of the nucleic acid sequences set forth in SEQ ID NOs: 29-47.

In some embodiments, the disclosure provides nucleic acid sequences, each of which encodes one or more of the open reading frames (ORFs) provided herein. Exemplary nucleic sequences that encode such ORFs are provided in SEQ ID NOs: 112-116, 122, and 123. However, it should be appreciated that variants of the ORF sequences provided herein are also contemplated. In some embodiments, the ORF is encoded by a nucleic acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the nucleic acid sequences set forth in any one of SEQ ID NOs: 112-116, 122, and 123. In some embodiments, the ORF is encoded by a nucleic acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, or more mutations (e.g., at least 50, 100, 200, 300, 400, 500, or 600) compared to any one of the nucleic acid sequences set forth in SEQ ID NOs: 112-116, 122, and 123. In some embodiments, the nucleic acid sequence encoding the ORF comprises a nucleic acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 200, at least 300, at least 400, at least 500, at least 600, at least 700, at least 800, at least 900, at least 1000, at least 1100, at least 1200, at least 1300, at least 1400, at least 1500, at least 1600, or at least 1700 identical contiguous nucleotides as compared to any one of the nucleic acid sequences set forth in SEQ ID NOs: 112-116, 122, and 123.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one preferred embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In a preferred embodiment, the mRNA has both a cap on the 5′ end and a 3′ polyA tail which determine ribosome binding, initiation of translation and stability mRNA in the cell.

A conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (size can be 50-5000 T, or after PCR by any other method, including, but not limited to, enzymatic addition, DNA ligation, or in vitro recombination. PolyA tails also provide stability to RNAs and reduce their degradation. Generally, the length of a polyA tail positively correlates with the stability of the transcribed RNA. In one embodiment, the polyA tail is between 100 and 5000 adenosines. In some embodiments, the polyA tail is at least 150 (e.g., at least 150, 180, or 200 adenosines) adenosines. In some embodiments, the polyA tail is from 100 to 200, 100 to 300, 100 to 400, 100 to 500, 100 to 600, 100 to 700, 100 to 800, 100 to 900, 100 to 1000, 200 to 300, 200 to 400, 200 to 500, 200 to 600, 200 to 700, 200 to 800, 200 to 900, 200 to 1000, 300 to 400, 300 to 500, 300 to 600, 300 to 700, 300 to 800, 300 to 900, 300 to 1000, 400 to 500, 400 to 600, 400 to 700, 400 to 800, 400 to 900 400 to 1000, 500 to 600 500 to 700, 500 to 800, 500 to 900, 500 to 1000, 600 to 700, 600 to 800, 600 to 900, 600 to 1000, 700 to 800, 700 to 900, 700 to 1000, 800 to 900, 800 to 1000, or 900 to 1000 adenosines

PolyA tails of RNAs can be further extended following in vitro transcription with the use of a polyA polymerase, such as E. coli polyA polymerase (E-PAP). Additionally, the attachment of different chemical groups to the 3′ end can increase mRNA stability. Such attachment can contain modified/artificial nucleotides, aptamers and other compounds. For example, ATP analogs can be incorporated into the polyA tail using polyA polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs provided herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

The nucleic acid sequences coding for the desired molecules (e.g., CARs) can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned. Exemplary vectors include, without limitation cosmids, plasmids (e.g., naked or contained in liposomes), retrotransposons (e.g. piggyback, sleeping beauty), and viruses (e.g., lentiviruses, retroviruses, adenoviruses, and adeno-associated viruses) that incorporate any of the nucleic acids provided herein.

The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lenti viral vectors have the added advantage over vectors derived from onco-retroviruses such as murine leukemia viruses in that they can transduce non-proliferating cells, such as hepatocytes. They also have the added advantage of low immunogenicity. In another embodiment, the desired CAR can be expressed in the cells by way of transposons. In another embodiment, the desired CAR can be express in the cells by way of homology-directed recombination.

The vectors can be suitable for replication and integration into eukaryotes. Typical cloning vectors contain transcription and translation terminators, initiation sequences, and promoters useful for regulation of the expression of the desired nucleic acid sequence. The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector. The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, retrovirus vectors are used. A number of retrovirus vectors are known in the art. In some embodiments, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Factor-1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence with which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. In some embodiments, the promoter is an EF-1a promoter.

In order to assess the expression of a CAR polypeptide or portions thereof, the expression vector(s) to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic resistance genes, such as neo and the like. Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity, antibiotic resistance or fluorescence. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector(s), the vector(s) can be readily introduced into a host cell, e.g., mammalian, bacterial, yeast, or insect cell by any method in the art. For example, the expression vector(s) can be transferred into a host cell by physical, chemical, or biological means. In some embodiments, the host cell is a T cell. Physical methods for introducing a polynucleotide into a host cell include electroporation, mechanical membrane disruption (e.g., cell squeezing or nanoparticle-based delivery), calcium phosphate precipitation, lipofection, particle bombardment, microinjection, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is electroporation.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about-20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### RNA Transfection

In some embodiments, the modified T cells of the invention are modified through the introduction of RNA (e.g., an mRNA comprises a sequence encoding a CAR as described herein). In some embodiments, an in vitro transcribed RNA CAR can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, eDNA, synthetic DNA sequence or any other appropriate source of DNA. The desired template for in vitro transcription is the CAR of the present invention.

RNA can be introduced into target cells using any of a number of different methods, for instance, commercially available methods which include, but are not limited to, electroporation (Amaxa® Nucleofector-II®) (Amaxa Biosystems, Cologne, Germany)), (ECM 830 (BTX) (Harvard Instruments, Boston, Mass.) or the Gene Pulser II® (BioRad, Denver, Colo.), Multiporator® (Eppendort, Hamburg Germany), mechanical membrane disruption (e.g., cell squeezing, see U.S. Pat. Pub. No. 2014/287509A1), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

Disclosed herein are methods for producing an in vitro transcribed RNA CAR. The present invention also includes a CAR encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-400, 50-2000 bases, 150-400 bases, or 150-2000 bases in length. RNA so produced can efficiently transfect different kinds of cells. In one aspect, the template includes sequences for the CAR.

In one aspect the anti-BCMA CAR is encoded by a messenger RNA (mRNA). In one aspect the mRNA encoding the anti-BCMA CAR is introduced into an immune effector cell, e.g., a T cell or a NK cell, for production of a CAR-expressing cell (e.g., CAR T cell or CAR-expressing NK cell).

In one embodiment, the in vitro transcribed RNA CAR can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA. cDNA, synthetic DNA sequence or any other appropriate source of DNA. The desired temple for in vitro transcription is a CAR of the present invention. For example, the template for the RNA CAR comprises an extracellular region comprising a single chain variable domain of an anti-tumor antibody; a hinge region, a transmembrane domain (e.g., a transmembrane domain of CD8a); and a cytoplasmic region that includes an intracellular signaling domain, e.g., comprising the signaling domain of CD3zeta and/or the signaling domain of CD28, 41BB and/or OX40.

In one embodiment, the DNA to be used for PCR contains an open reading frame. The DNA can be from a naturally occurring DNA sequence from the genome of an organism. In one embodiment, the nucleic acid can include some or all of the 5′ and/or 3′ untranslated regions (UTRs), e.g., any of the untranslated regions provided herein. The nucleic acid can include exons and introns. In one embodiment, the DNA to be used for PCR is a human nucleic acid sequence. In another embodiment, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In one embodiment, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any high-fidelity DNA polymerase useful for PCR can be used in the methods disclosed herein. The reagents and polymerase are commercially available from a number of sources.

### Genetically Modified Immune Cells

In some embodiments, the CAR sequence(s) (e.g., nucleic acid sequence(s) encoding a CAR as described herein) are delivered into cells (e.g., T cells, stem cells, or NK cells) using a retroviral or lentiviral vector. CAR-expressing retroviral and lentiviral vectors can be delivered into different types of eukaryotic cells as well as into tissues and whole organisms using transduced cells as carriers or cell-free local or systemic delivery of encapsulated, bound or naked vectors. The method used can be for any purpose where stable expression is required or sufficient.

In another embodiment, the desired CAR can be expressed in the cells (e.g., T cells or NK cells) by way of transposons.

The disclosed methods can be applied to the modulation of immune cell (e.g., T cell or NK cell) activity in basic research and therapy, in the fields of cancer, stem cells, acute and chronic infections, and autoimmune diseases, including the assessment of the ability of the genetically modified T cell or NK cell to kill a target cell, e.g., a target cancer cell, vector, making it possible to individually regulate the expression level. For example, varying of different intracellular effector/costimulator domains on multiple chimeric receptors in the same cell allows determination of the structure of the receptor combinations which assess the highest level of cytotoxicity against multi-antigenic targets, and at the same time lowest cytotoxicity toward normal cells.

Cloning of cells is not necessary because of the efficiency of transduction of the CAR with lentiviral vectors or onco-retroviral vectors, which can stably and uniformly modify the entire lymphocyte population.

### Sources of Immune Cells

Prior to expansion and genetic modification of the immune cells (e.g., T cells) of the invention, a source of immune cells (e.g., T cells) is obtained from a subject. Immune cells (e.g., T cells) can be obtained from a number of sources, including peripheral blood mononuclear cells, bone marrow, lymph node tissue, cord blood, thymus tissue, tissue from a site of infection, ascites, pleural effusion, spleen tissue, and tumors. The immune cells (e.g., T cells) may also be generated from induced pluripotent stem cells or hematopoietic stem cells or progenitor cells. In some embodiments of the present invention, any number of immune cell lines, including but not limited to T cell and NK cell lines, available in the art, may be used. In some embodiments of the present invention, immune cells (e.g., T cells) can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, NK cells, other nucleated white blood cells, red blood cells, and platelets. In some embodiments, the cells collected by apheresis may be washed to remove the plasma fraction and to place the cells in an appropriate buffer or media for subsequent processing steps. In some embodiments of the invention, the cells are washed with phosphate buffered saline (PBS). In an alternative embodiment, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe® 2991 cell processor, the Baxter® CytoMate®, or the Haemonetics® Cell Saver 5®) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In another embodiment, immune cells (e.g., T cells) are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4⁺, CD25⁺, CD62L^{hi}, GITR⁺, and FoxP3⁺.

Alternatively, in certain embodiments. T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in some embodiments, a concentration of 2 billion cells/ml is used. In some embodiments, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8⁺T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4⁺ T cells express higher levels of CD28 and are more efficiently captured than CD8⁺ T cells in dilute concentrations. In some embodiments, the concentration of cells used is 5×10⁶/ml. In other embodiments, the concentration used can be from about 1×10⁵/ml to 1×10⁶/ml, and any integer value in between.

In other embodiments, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen. In certain embodiments, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In some embodiments a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate. and FK506, antibodies, or other immunoablative agents such as CAMPATH®, anti-CD3 antibodies, Cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991: Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH®. In another embodiment, the cells are isolated prior to and can be frozen for later use for treatment following B-cell ablative therapy such as agents that react with CD20, e.g., RITUXAN®.

In a further embodiment of the present invention, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

Whether prior to or after genetic modification of the T cells to express a desirable CAR, the T cells can be activated and expanded generally using methods as described, for example, in U.S. Pat. No. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention are expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8): 3975-3977, 1998; Haanen et al., J. Exp. Med. 190(9): 13191328, 1999; Garland et al., J. Immunol Meth. 227(1-2):53-63, 1999).

In certain embodiments, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In some embodiments, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In some embodiments, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts. In some embodiments, a 1:1 ratio of each antibody bound to the beads for CD4+ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular embodiment an increase of from about 1 to about 3 fold is observed as compared to the expansion observed using a ratio of 1:1. In some embodiments, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1. In one particular embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In one preferred embodiment, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In some embodiments, a ratio of particles to cells of 1:1 or less is used. In one particular embodiment, a preferred particle: cell ratio is 1:5. In further embodiments, the ratio of particles to cells can be varied depending on the day of stimulation. For example, in some embodiments, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular embodiment, the ratio of particles to cells is 1:1 on the first day of stimulation and adjusted to 1:5 on the third and fifth days of stimulation. In another embodiment, particles are added on a daily or every other day basis to a final ratio of 1:1 on the first day, and 1:5 on the third and fifth days of stimulation. In another embodiment, the ratio of particles to cells is 2:1 on the first day of stimulation and adjusted to 1:10 on the third and fifth days of stimulation. In another embodiment, particles are added on a daily or every other day basis to a final ratio of 1:1 on the first day, and 1:10 on the third and fifth days of stimulation. One of skill in the art. will appreciate that a variety of other ratios may be suitable for use in the present invention. In particular, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In some embodiments the cells (for example, 104 to 109 T cells) and beads (for example, DYNABEADS® M-450 CD3/CD28 T paramagnetic beads at a ratio of 1:1) are combined in a buffer, preferably PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in some embodiments, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28− negative T cells. Such populations of cells may have therapeutic value and would be desirable to obtain in certain embodiments. For example, using high concentration of cells allows more efficient selection of CD8+ T cells that normally have weaker CD28 expression.

In some embodiments of the present invention, the mixture may be cultured for several hours (about 3 hours) to about 14 days or any hourly integer value in between. In another embodiment, the mixture may be cultured for 21 days. In some embodiments of the invention the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15. (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGF-β, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V. DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

T cells that have been exposed to varied stimulation times may exhibit different characteristics. For example, typical blood or apheresed peripheral blood mononuclear cell products have a helper T cell population (¾, CD4+) that is greater than the cytotoxic or suppressor T cell population (Tc, CD8+). Ex vivo expansion of T cells by stimulating CD3 and CD28 receptors produces a population of T cells that prior to about days 8-9 consists predominately of 34 cells, while after about days 8-9, the population of T cells comprises an increasingly greater population of Tc cells. Accordingly, depending on the purpose of treatment, infusing a subject with a T cell population comprising predominately of TH cells may be advantageous. Similarly, if an antigen-specific subset of Tc cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

### Therapeutic Application

In some embodiments, the present invention encompasses a cell (e.g., T cell) modified to express a CAR that combines an antigen recognition domains (e.g., an scFv specific for BCMA), a transmembrane domain (e.g., a CD8 transmembrane domain) and a cytoplasmic domain (e.g., an intracellular domain of CD3-zeta, CD28, OX40, 4-1BB, or any combinations thereof). Therefore, in some instances, the transduced immune cell (e.g., T cell) can elicit a CAR-mediated immune (e.g., T-cell) response. In some embodiments, the invention provides the use of a CAR to redirect the specificity of a primary T cell to a tumor antigen. Thus, in some embodiments, the present invention also provides a method for stimulating a T cell-mediated immune response to a target cell population or tissue in a mammal comprising the step of administering to the mammal a T cell that expresses a CAR, wherein the CAR comprises a binding moiety that specifically interacts with a predetermined target (e.g., BCMA), a zeta chain portion comprising for example the intracellular domain of human CD3zeta, and a costimulatory signaling region. In some embodiments, the present invention includes a type of cellular therapy where T cells are genetically modified to express a CAR and the CAR T cell is infused to a recipient in need thereof. The infused cell is able to kill tumor cells in the recipient. Unlike antibody therapies, CAR T cells are able to replicate in vivo resulting in long-term persistence that can lead to sustained tumor control.

Without wishing to be bound by any particular theory, the anti-tumor immunity response elicited by the CAR-modified T cells may be an active or a passive immune response. In addition, the CAR mediated immune response may be part of an adoptive immunotherapy approach in which CAR-modified T cells induce an immune response specific to the antigen binding moiety in the CAR. For example, BCMA-specific CAR T cells elicit an immune response specific against cells expressing BCMA. While the data disclosed herein specifically disclose lentiviral vectors comprising anti-BCMA scFv, a CD8 transmembrane domain, and 41BB, CD28 and CD3zeta signaling domains, the invention should be construed to include any number of variations for each of the components of the construct as described elsewhere herein. That is, the invention includes the use of any antigen binding moiety in the CAR to generate a CAR-mediated T-cell response specific to the antigen binding moiety. For example, the antigen binding moiety in the CAR of the invention can target a tumor antigen for the purposes of treating cancer. In some embodiments, the antigen binding moiety portion of the CAR of the invention is designed to treat a particular cancer, such as multiple myeloma.

The CAR-modified T cells of the invention may also serve as a type of vaccine for ex vivo immunization and/or in vivo therapy in a mammal. Preferably, the mammal is a human.

With respect to ex vivo immunization, at least one of the following occurs in vitro prior to administering the cell into a mammal: i) expansion of the cells, ii) introducing a nucleic acid encoding a CAR to the cells, and/or iii) cryopreservation of the cells. Ex vivo procedures are well known in the art and are discussed more fully below. Briefly, cells are isolated from a mammal (preferably a human) and genetically modified (e.g., transduced or transfected in vitro) with a vector expressing a CAR disclosed herein. The CAR-modified cell can be administered to a mammalian recipient to provide a therapeutic benefit. The mammalian recipient may be a human and the CAR-modified cell can be autologous with respect to the recipient. Alternatively, the cells can be allogeneic, syngeneic or xenogeneic with respect to the recipient.

A procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells. In addition to using a cell-based vaccine in terms of ex vivo immunization, the present invention also provides compositions and methods for in vivo immunization to elicit an immune response directed against an antigen in a patient.

Generally, the cells activated and expanded as described herein may be utilized in the treatment and prevention of diseases that arise in individuals who are immunocompromised, such as individuals having cancer. In particular, the CAR-modified T cells of the invention are used in the treatment of multiple myeloma. In certain embodiments, the cells of the invention are used in the treatment of patients at risk for developing multiple myeloma.

The CAR-modified immune cells (e.g., CAR T cells) of the present invention, or a composition comprising such cells, may be used, or may be administered to a subject in need thereof, to provide anti-tumor immunity; to treat or prevent cancer; to treat or prevent autoimmune condition; or to treat or prevent an allergic condition. In some embodiments, the cancer is multiple myeloma, Hodgkin lymphoma, non-Hodgkin lymphoma, a leukemia, or glioblastoma. In some embodiments, the autoimmune condition is myasthenia gravis, systemic lupus erythematosus, rheumatoid arthritis, pemphigus, psoriasis, inflammatory bowel disease, celiac sprue, pernicious anemia, idiopathic thrombocytopenia purpura, scleroderma, Graves' disease, Sjögren syndrome, Goodpasture syndrome, or type 1 diabetes. In some embodiments, the allergic condition is anaphylaxis, asthma, food allergy, stinging insect allergy, drug allergy, allergic rhinitis, urticaria, angioedema, eczema, atopic dermatitis, contact dermatitis, and eosinophilic esophagitis.

The CAR-modified immune cells (e.g., CAR T cells) of the present invention may be administered either alone, or as a composition (e.g., a pharmaceutical composition) in combination with diluents and/or with other components such as IL-2 or other cytokines or cell populations. Briefly, pharmaceutical compositions of the present invention may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”. “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the CAR-modified immune cells (e.g., CAR T cells) described herein may be administered at a dosage of 10⁴ to 10⁹ cells/kg body weight, preferably 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319: 1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated immune (e.g., T cells) to a subject and then subsequently redraw blood (or have an apheresis performed), activate T cells therefrom according to the present invention, and reinfuse the patient with these activated and expanded T cells. This process can be carried out multiple times every few weeks. In certain embodiments. T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocol may serve to select out certain populations of T cells.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In some embodiments, the immune cell (e.g., T cell) compositions of the present invention are administered to a patient by intradermal or subcutaneous injection. In another embodiment, the immune cell (e.g., T cell) compositions of the present invention are preferably administered by i.v. injection. The compositions of immune cells (e.g., T cells) may be injected directly into a tumor, lymph node, or site of disease.

In certain embodiments of the present invention, cells activated and expanded using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAMPATH (alemtuzumab), anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766). Strategies for CAR T cell dosing and scheduling have been discussed (Ertl et al., 2011, Cancer Res, 71:3175-81; Junghans, 2010, Journal of Translational Medicine, 8:55).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described herein may be more fully understood, the following examples are set forth. The synthetic examples described in this application are offered to illustrate the compounds and methods provided herein and are not to be construed in any way as limiting their scope.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

### Example 1: Production of Functional CAR T Cells from an Inventive mRNA Construct

Human CAR T cells were produced by use of an inventive mRNA construct that encoded an inventive CAR protein. The CAR T cells were observed to be viable, to bind BCMA, and to kill BCMA+ tumor cells.

An inventive mRNA construct comprising the nucleotide sequence of SEQ ID: 31 was generated by in vitro transcription from a DNA plasmid. In vitro transcription was performed by T7 RNA polymerase from a linearized plasmid template and a polyadenine tail of about 150 adenine nucleotides was added enzymatically. A 7-methylguanosine cap was incorporated at the 5′ end of the mRNA during the co-transcriptional mRNA synthesis.

The inventive mRNA construct comprised, from 5′ to 3′: a 5′ cap, a 5′ UTR described as SEQ ID: 74, an open reading frame (ORF) described as SEQ ID: 112, a 3′ UTR described as SEQ ID: 92, and a 3′ polyadenine tail of 150 adenine units or more. The ORF encoded an inventive CAR protein with the amino acid sequences of SEQ ID: 21.

To prepare CAR T cells from mRNA constructs, lymphocytes were obtained by apheresis from a healthy human donor. From these lymphocytes, CD8+ T cells were positively selected with paramagnetic microbeads conjugated to an anti-CD8 antibody. This yielded cells that were 95% CD8+ T cells and 95% viable. These enriched CD8+ T cells were expanded by incubation at 37° C. with 5% CO₂ in the presence of anti-CD3 antibody (clone OKT3) for about 14 days. The cells were resuspended in P3 transfection buffer (Lonza) and transfected with the mRNA construct by electroporation (4D) Nucleofector®, Lonza) according to manufacturer's instructions. Cells were then returned to culture in a standard medium containing IL-15 for about 8 hours.

CAR T cells obtained from the above-described process were tested for viability, CAR protein expression, BCMA binding, and cytotoxicity, i.e., the ability to kill BCMA+ myeloma (tumor) cells. Viability, CAR expression, and BCMA binding were determined by flow cytometry on a GUAVA® EASYCYTE® 12HT Flow cytometer (EMD Millipore). To test viability, a sample of the CAR T cells was mixed with propidium iodide and run on the flow cytometer with electronic gating on fluorescence in the near infrared channel. To test CAR protein expression and BCMA binding, a sample of the CAR T cells was incubated with 0.4 ug/mL of phycoerythrin (PE)-conjugated BCMA (Recombinant Human TNFRSF17 protein, Fc/His-tagged, R-PE labeled; Creative Biomart, Shirley, NY). CAR expression was assessed on the flow cytometer with electronic gating on fluorescence in the yellow channel to detect presence or absence of emission from BCMA-PE on CAR-positive and CAR-negative cells. BCMA binding was determined by measuring the intensity of fluorescence in the yellow channel to determine the relative quantity of BCMA-PE bound to labelled CAR-positive cells. For the viability, expression, and BCMA binding assays, CD8+ T cells electroporated without mRNA (CAR-negative T Cells) were tested as a parallel control.

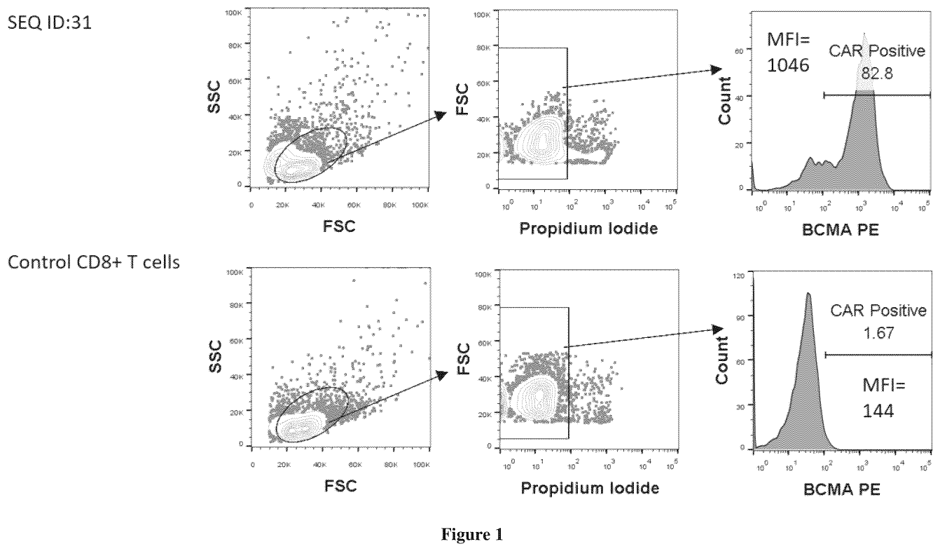

To test the capacity of the CAR T cells to kill BCMA+ myeloma cells, the CAR T cells were co-incubated with BCMA+ myeloma cell line expressing green fluorescent protein (MM.1S-GFP). Aliquots of 50,000 MM.1S-GFP tumor cells were placed in wells of a 96-well plate. Between about 2500 and 50,000 CAR T cells were added to each well to obtain various effector:target ratios (i.e., ratios of CAR T cells to BCMA+ myeloma cells) that were between about 1:1 and 1:20. Following overnight incubation, propidium iodide was used to stain dead cells. Viable target cells were identified, and cell density was determined by flow cytometry. The degree of myeloma cell killing by the CAR T cells was calculated by comparison to the number of myeloma cells in wells concurrent control wells that did not contain CAR T cells. The results are reflected in FIG. 1 and Table 2, which show an assessment of cell viability, CAR expression, BCMA binding by CAR T cells generated with the CAR of SEQ ID: 31 or a control (no mRNA) at 24 hours post transfection, as well as the percentage of killing of BCMA+ myeloma (tumor) cells.

TABLE 2
Assessment of cell viability, CAR expression, BCMA binding
by CAR T cells generated with the CAR of SEQ ID: 31, and
the percentage of killing of BCMA+ myeloma (tumor) cells.

| | | CAR+ | BCMA | |
|---|---|---|---|---|
| | | Cells | binding | % |
| | Viability * | (%) * | (MFI) * | Killing ** |
| CAR T cells | 83.9 | 86.6 | 1333 | 94.4 |
| Control CD8+ T cells | 90.9 | 1.8 | 20 | 0 |
| (electroporated without | | | | |
| mRNA) | | | | |

* Measured at 4 hours post-transfection.
** 1:10 Effector to Target Ratio Used in Killing Assay (killing assay was incubated for 4 days).

In conclusion, an mRNA construct was used to produce CAR T cells. The mRNA construct encoded an inventive CAR protein. The CAR T cells demonstrated high viability, CAR protein expression, BCMA binding, and killing of BCMA+ myeloma (tumor) cells. CD8+ T cells electroporated without the mRNA construct showed essentially no CAR expression, essentially no BCMA binding, and no killing of tumor cells.

### Example 2: Amino Acid Substitutions in CAR Complementarity-Determining Regions

Various amino acid substitutions were introduced into the complementarity-determining regions (CDRs) of anti-BCMA CARs in order to generate CARs with improved properties. Fortuitously, these modifications substantially improved BCMA binding and tumor killing by CAR T cells compared to the CAR T cells and the respective CAR used in the clinical trial of Ali et al. (2016), i.e., of SEQ ID: 19.

Numerous CAR-encoding mRNA constructs were prepared using SEQ ID: 29 as a reference sequence. The new constructs varied with respect to one or more amino acid residues in one or more CDRs.

Below, Table 3 provides mRNA constructs described in this example. Table 3 indicates the respective parent sequence and sequence fragments included in each of the three heavy-chain and three light-chain CDRs: CDRH1, CDRH2, CDRH3, CDRL1, CDRL2, and CDRL3. Portions of the CARs that correspond or substantially correspond to the scFv regions of the CARs are referred to as “New scFv Sequences” in Table 3.

TABLE 3
Listing of sequence identification numbers (SEQ ID NOs) for “new scFv” sequences,
including their parent sequence and corresponding CDR sequences. An asterisk (*)
indicates an amino acid substitution with respect to the parent sequence and is not part
of the sequence ID.

| | Parent | | | | | | |
|---|---|---|---|---|---|---|---|
| | sequence | | | | | | |
| New scFv | of scFv | HEAVY | | | LIGHT | | |
| Sequence | framework | CDRH 1 | CDRH 2 | CDRH 3 | CDRL 1 | CDRL 2 | CDRL 3 |
| 48 | 48 | 1 | 4 | 7 | 10 | 12 | 16 |
| 57 | 57 | 1 | 4 | 7 | 10 | 12 | 16 |
| 60 | 60 | 1 | 4 | 7 | 10 | 12 | 16 |
| 62 | 57 | 3* | 4 | 7 | 10 | 12 | 16 |
| 63 | 57 | 1 | 4 | 8* | 10 | 12 | 16 |
| 64 | 57 | 1 | 4 | 7 | 11* | 14* | 17* |
| 65 | 57 | 1 | 4 | 7 | 11* | 14* | 18* |
| 66 | 57 | 2* | 6* | 9* | 10 | 12 | 16 |
| 67 | 57 | 3* | 5* | 8* | 10 | 12 | 16 |
| 68 | 57 | 3* | 4 | 9* | 10 | 12 | 18* |
| 69 | 57 | 2* | 6* | 9* | 11* | 13* | 17* |
| 70 | 57 | 1 | 4 | 7 | 10 | 15* | 16 |

For each of the mRNA constructs, CAR T cells were prepared by transfection into human CD8+ cells, as described in Example 1. The CAR T cells made from each mRNA construct were then tested 3 days after electroporation for BCMA binding and tumor killing, as described in Example 1. Results are summarized in Table 4, shown below.

TABLE 4
BCMA binding results and % of human myeloma cells killed using
CAR T cells expressing the corresponding scFv portions.

| New scFv | BCMA Binding | Killing* |
|---|---|---|
| Sequence | (arbitrary units) | (% killed) |
| SEQ ID: 48 | 100 (standard | 46% |
| | of reference) | |
| SEQ ID: 57 | 343 | 85% |
| SEQ ID: 60 | 381 | 83% |
| SEQ ID: 62 | 189 | 3% |
| SEQ ID: 63 | 439 | 78% |
| SEQ ID: 64 | 496 | 26% |
| SEQ ID: 65 | 173 | 86% |
| SEQ ID: 66 | 269 | 81% |
| SEQ ID: 67 | 310 | 0% |
| SEQ ID: 68 | 446 | 7% |
| SEQ ID: 69 | 230 | 77% |
| SEQ ID: 70 | 404 | 83% |

*Effector:target ratio of 1:2.

Thus, several attempts to modify the CDRs were unsuccessful, as they reduced BCMA binding. However, certain CARs, e.g. those comprising sequences of SEQ ID: 57, SEQ ID: 60, SEQ ID: 65, SEQ ID: 66, SEQ ID: 69, and SEQ ID: 70, were not only substantially modified in their CDR residues compared to the initial CAR of SEQ ID: 19, but also provided dramatically better CAR expression and killing of human myeloma cells.

### Example 3. Humanization by Amino Acid Substitutions in CAR scFv Framework Regions

Various amino acid substitutions were introduced in the scFv framework regions of a CAR in order to humanize the CAR. Fortuitously, these modifications substantially improved CAR T cell BCMA binding and tumor killing compared to the CAR T cells of the non-humanized CAR used in the clinical trial of Ali et al (2016), i.e., of SEQ ID: 19.

Numerous CAR-encoding mRNA constructs were prepared using the mRNA construct of SEQ ID: 29 as a starting point (which encodes a CAR of sequence SEQ ID: 19 and comprises the scFv of sequence SEQ ID: 48). These constructs encoded the same or substantially the same amino acid residues in the scFv complementary-determining regions (CDRs) but varied with respect to the amino acid residues in the scFv framework regions. In making these variations, the experimenter's intent was to humanize the scFv framework regions without reducing BCMA-binding and tumor-killing properties of CAR T cells that express the CAR. For each of the mRNA constructs, CAR T cells were prepared by transfection into human CD8+ cells, substantially as described in Example 1. The CAR T cells made from each mRNA construct were then tested 3 days after electroporation for BCMA binding and/or tumor killing, as substantially described in Example 1.

In a first iteration of experiments, various modifications were made to scFv framework residues of the CAR, with results shown in Table 5:

TABLE 5
BCMA binding results using CAR T cells expressing the corresponding
scFv sequences having modifications to framework residues.

| scFv | BCMA Binding |
|---|---|
| Sequence | (relative to SEQ ID: 48) |
| SEQ ID: 48 | 100 (standard of reference) |
| SEQ ID: 54 | 141 |
| SEQ ID: 49 | 76 |
| SEQ ID: 50 | 51 |
| SEQ ID: 51 | 54 |
| SEQ ID: 52 | 82 |
| SEQ ID: 53 | 103 |
| SEQ ID: 55 | 12 |

Thus, several attempts to humanize the scFv framework were unsuccessful, as they reduced BCMA binding. However, a CAR comprising the scFv of SEQ ID: 54 provided a 41% improvement in CAR T cell BCMA binding over an otherwise comparable CAR comprising the reference scFv of SEQ ID: 48. Therefore, the scFv of SEQ ID: 54 was selected as a lead for further optimization. In a second iteration of experiments, further and various modifications were made to the scFv framework residues of the CAR, with the following results for BCMA binding, shown in Table 6:

TABLE 6
BCMA binding results using CAR T cells expressing
the corresponding scFv sequences having further
modifications to framework residues.

| scFv | BCMA Binding |
|---|---|
| Sequence | (relative to SEQ ID: 54) |
| SEQ ID: 54 | 100 (standard |
| | of reference) |
| SEQ ID: 56 | 58 |
| SEQ ID: 57 | 124 |
| SEQ ID: 58 | 40 |
| SEQ ID: 59 | 79 |

Several attempts to humanize the scFv framework were unsuccessful, as they reduced BCMA binding. However, the scFv of SEQ ID: 57 provided a further 24% improvement in CAR T cell BCMA binding over the scFv of SEQ ID: 54. Therefore, the CAR comprising the scFv of SEQ ID: 57 was selected as a lead for further optimization.

In a third iteration of experiments, the CAR comprising scFv of SEQ ID: 57 was compared: (1) to CARs comprising the initial scFv of SEQ ID: 48; (2) to an scFv of SEQ ID: 60, which was further modified from that of SEQ ID: 57; and (3) to an scFv of SEQ ID: 61, which was developed by an independent method from the scFv of SEQ ID: 48 (Antibody Humanization by a Single Cycle of CDR-Grafting, Hu et al. in Ricin Toxin; Bentham Science Publishers Eds. J W Cherwonwogrodzky). The following results, shown in table 7, were seen for BCMA binding and tumor killing:

TABLE 7
BCMA binding results and % of human myeloma cells killed using
CAR T cells expressing the corresponding scFv portions.

| scFv Sequence | BCMA Binding* | Killing** (% killed) |
|---|---|---|
| SEQ ID: 48 | 100 (standard | 46% |
| | of reference) | |
| SEQ ID: 57 | 460 | 85% |
| SEQ ID: 60 | 382 | 83% |
| SEQ ID: 61 | 34 | Not tested. |

**Effector:target ratio of 1:10.

Lastly, to measure the amount of humanization achieved, the amino acids in the framework regions of the scFv of each of the above sequences were compared to those of that sequence's closest germline neighbor as identified from sequences in the Immunogenetics Database (IMGT) (IMGT®, the international ImMunoGeneTics information System® 25 years on. Lefranc M-P et al., Nucleic Acids Res. 2015 January; 43:D413-22. The mean amino acid identity to the closest germline neighbor was 78% for SEQ ID: 48, 84% for SEQ ID: 57, and 88% for SEQ ID: 60. Thus, at the conclusion of this series of sequence modifications and assays, the CARs containing scFv of SEQ ID: 57 and SEQ ID: 60 were not only substantially humanized in their scFv framework residues compared to the initial CAR of SEQ ID: 19, but also provided CAR T cells with dramatically better BCMA binding and tumor killing.

### Example 4. CARs Combining Humanized Framework and CDR Regions

Experiments were carried out to test various combinations of the humanized scFv framework and CDR sequences described in Examples 2 and 3. The purpose was to determine which of various combinations would provide CAR T cells with superior BCMA binding and tumor killing. Provided below is a reference table for the sequences of this example. For each combination shown in the table, CAR T cells were prepared and tested substantially as described in Example 1. For comparison, CAR T cells were also prepared with a CAR protein corresponding to that of Ali et al (2016), i.e., of SEQ ID: 19.

Table 8 indicates the respective parent sequence and sequence fragments included in each of the three heavy-chain and three light-chain CDRs: CDRH1, CDRH2, CDRH3, CDRL1, CDRL2, and CDRL3.

TABLE 8
Listing of sequence identification numbers (SEQ ID NOs) for “Full Length Car”
sequences, including their parent sequence of scFv framework and corresponding CDR
sequences. An asterisk (*) indicates an amino acid substitution with respect to the
parent sequence and is not part of the sequence ID. CAR Sequences SEQ ID NOs:
21, 23, and 25 are versions of SEQ ID NOs: 20, 22, and 24, respectively, that further
comprise a 41BB intracellular costimulatory domain.

| | Parent | | | | | | |
|---|---|---|---|---|---|---|---|
| Full Length | sequence of | HEAVY | | | LIGHT | | |
| CAR Sequence | scFv framework | CDRH1 | CDRH2 | CDRH3 | CDRL1 | CDRL2 | CDRL3 |
| 19 | 48 | 1 | 4 | 7 | 10 | 12 | 16 |
| 28 | 60 | 1 | 4 | 7 | 10 | 12 | 16 |
| 20 | 60 | 2* | 6* | 9* | 11* | 14* | 17* |
| 21 | 60 | 2* | 6* | 9* | 11* | 14* | 17* |
| 22 | 60 | 2* | 6* | 9* | 11* | 14* | 18* |
| 23 | 60 | 2* | 6* | 9* | 11* | 14* | 18* |
| 24 | 60 | 1 | 4 | 8* | 10 | 15* | 17* |
| 25 | 60 | 1 | 4 | 8* | 10 | 15* | 17* |

CAR T cells made from each mRNA construct were tested about 3 days after electroporation for BCMA binding and tumor killing. The results of which are indicated in Table 9:

TABLE 9
BCMA binding results and % of human myeloma cells killed using
CAR T cells expressing the corresponding CAR portions.

| CAR Sequence | BCMA Binding* | Killing** (% killed) |
|---|---|---|
| SEQ ID: 19 | 100 (standard | 46% (prior |
| | of reference) | experiment) |
| SEQ ID: 28 | 377 | 83% |
| SEQ ID: 20 | 189 | 84% |
| SEQ ID: 21 | 439 | 69% |
| SEQ ID: 22 | 496 | 73% |
| SEQ ID: 23 | 173 | 67% |
| SEQ ID: 24 | 269 | 74% |
| SEQ ID: 25 | 310 | 60% |

Thus, the humanized constructs of SEQ ID: 28, SEQ ID: 20, SEQ ID: 21, SEQ ID: 22, SEQ ID: 23, SEQ ID: 24, and SEQ ID: 25 provided CAR T cells with superior BCMA binding and killing of human myeloma cells, as compared to SEQ ID: 19.

The degree of humanization of the combined framework and CDR regions in the scFv of these constructs was measured using the method of Lazar (A molecular immunology approach to antibody humanization and functional optimization, Lazar GA. et al. Molecular Immunology (2007) 44:1986) to determine Human String Content and Perfect 9mer scores. The results are summarized in Table 10:

TABLE 10
Degree of humanization for each indicated CAR amino acid sequence,
including the human string content and perfect 9mer score.

| Sequence | Human String Content | Perfect 9mer Score |
|---|---|---|
| SEQ ID: 19 | 72% | 13 |
| SEQ ID: 28 | 81% | 69 |
| SEQ ID: 20 | 86% | 92 |
| SEQ ID: 21 | 86% | 92 |
| SEQ ID: 22 | 87% | 96 |
| SEQ ID: 23 | 87% | 96 |
| SEQ ID: 24 | 81% | 69 |
| SEQ ID: 25 | 81% | 69 |

The Human String Content and Perfect 9mer Scores showed that successful humanization of the parent sequence had been achieved. In conclusion, after an extensive series of iterative, empirical residue modifications to the scFv regions of a CAR, several CARs were obtained that were not only humanized in their scFv regions with respect to the CAR of SEQ ID: 19, but also provided CAR T cells with dramatically better BCMA binding and killing of myeloma cells.

### Example 5: Amino Acid Substitutions in CAR Costimulatory Domains

A controlled experiment was conducted to test whether inclusion or modification of certain intracellular costimulatory domains in the CAR protein improved expression of the CAR protein and improved BCMA binding and tumor killing by the corresponding CAR T cells.

Multiple mRNA constructs were prepared, wherein the CAR proteins they encoded comprised the same signal peptide, scFv, stalk, and transmembrane domains but differed by one or more amino acids in their costimulatory domains. For each of the mRNA constructs, CAR T cells were prepared by transfection into human CD8+ cells, substantially as described in Example 1. The CAR T cells made from each mRNA construct were then tested around 24 hours after electroporation for CAR expression and antitumor cytotoxicity, substantially as described in Example 1. The mRNA construct used in the clinical trial of Ali et al (2016) was tested as a control (SEQ ID: 29). The mRNA constructs and their expression results are shown below, in Table 11. For each costimulatory domain, the SEQ ID number and natural source of the sequence are shown.

TABLE 11
Relative expression and % of human myeloma cells killed using CAR
T cells expressing the corresponding CAR costimulatory domains.

| | Gene Source for | | |
|---|---|---|---|
| | Costimulatory | Relative | Killing* |
| Sequence | Domain | expression | (% killed) |
| SEQ ID: 29 | CD28-CD3z | 100 (standard | 50% |
| | | of reference) | |
| SEQ ID: 103 | CD28-CD3z | 136 | 52% |
| SEQ ID: 107 | CD3z | 206 | 64% |
| SEQ ID: 104 | 41BB-CD3z | 197 | 58% |
| SEQ ID: 105 | OX40-CD3z | 214 | 46% |
| SEQ ID: 106 | 41BB-OX40-CD3z | 217 | 43% |

*Effector:target ratio of 1:10.

Compared to constructs that comprised a CD28-CD3z domain, those that comprised CD3z only, 41BB-CD3z, OX40-CD3z, or 41BB-OX40-CD3z showed superior expression. This was unexpected, because the substitution of one naturally occurring costimulatory domain for another would not normally be expected to affect protein expression.

Constructs that comprised a costimulatory domain that was CD3z only or 41BB-CD3z also provided better killing of BCMA+ tumor cells. Despite their excellent expression, constructs that comprised OX40-CD3z or 41BB-OX40-CD3z showed inferior tumor killing compared to constructs that comprised a CD28-CD3z domain.

Thus, in a controlled experiment that tested variations in costimulatory regions, the constructs of SEQ ID: 107 and SEQ ID: 104 provided a combination of superior expression and tumor killing. Improved expression did not necessarily coincide with improved killing.

### Example 6: Substitution of Untranslated Regions in a CAR-Encoding mRNA Construct

An experiment was conducted to test how variation in the 5′ and/or 3′ untranslated regions of a CAR-encoding mRNA construct would affect CAR expression in human T cells.

Numerous mRNA constructs were prepared that encoded the same CAR protein (i.e., of SEQ ID: 59) but differed in the nucleotide sequences of their 5′ and/or 3′ untranslated regions (UTRs). These constructs were otherwise identical, e.g., with respect to the 5′ cap and polyadenine tail. The intention was to compare several constructs that incorporated different 5′ and 3′ UTR sequences that naturally occurred in one or more human genes, e.g., the CD3D and CD8B2 genes.

For one construct, a 218-nucleotide portion that occurs in the ORF of the GATA3 gene (i.e., SEQ ID NO: 92), not its 3′ UTR, was used as the 3′UTR and functioned particularly well.

For each of the mRNA constructs, CAR T cells were prepared substantially as described in Example 1. About 24 hours after transfection, CAR T cells made from each mRNA construct were tested for level of CAR expression substantially by the methods described in Example 1. The mRNA constructs and their expression results are shown below in Table 12. For each construct, the respective SEQ ID numbers and natural source of the sequence are shown.

TABLE 12
Relative expression of the CAR of SEQ ID NO: 59 (Y34) using
the indicated combination of 5′ and 3′ UTR sequences.
The sequence of SEQ ID NO: 92, which is the open reading frame
(ORF) of GATA3, worked particularly well to improve expression.

| | 5′ UTR | | 3′UTR | |
|---|---|---|---|---|
| | Sequence | | Sequence | |
| | (Natural | CAR ORF | (Natural | Relative |
| Construct | source) | Sequence | source) | expression* |
| A | SEQ ID: 77 | SEQ ID: 59 | SEQ ID: 87 | 100 |
| | (CD3D) | | (CD3D) | |
| B | SEQ ID: 75 | SEQ ID: 59 | SEQ ID: 87 | 146 |
| | (CD8B2) | | (CD3D) | |
| C | SEQ ID: 75 | SEQ ID: 59 | SEQ ID: 89 | 63 |
| | (CD8B2) | | (MYH8) | |
| D | SEQ ID: 74 | SEQ ID: 59 | SEQ ID: 86 | 103 |
| | (IgG) | | (AFP) | |
| E | SEQ ID: 74 | SEQ ID: 59 | SEQ ID: 87 | 165 |
| | (IgG) | | (CD3D) | |
| F | SEQ ID: 74 | SEQ ID: 59 | SEQ ID: 92 | 264 |
| | (IgG) | | (GATA3 ORF) | |
| G | SEQ ID: 74 | SEQ ID: 59 | SEQ ID: 84 | 136 |
| | (IgG) | | (IgG) | |
| H | SEQ ID: 74 | SEQ ID: 59 | SEQ ID: 89 | 70 |
| | (IgG) | | (MYH8) | |

*Mean fluorescence intensity, normalized to Construct A = 100.
IgG = IgG heavy chain, chromosome 14.

For the 5′ UTR, SEQ ID: 74 conferred best expression (compare Constructs A, B and E). For the 3′ UTR, SEQ ID: 92 conferred the best expression (compare Constructs E and F).

In conclusion, an experiment tested how variation in the 5′ and/or 3′ untranslated regions of a CAR-encoding mRNA construct would affect CAR expression in human T cells. Among several alternatives that appeared equally reasonable a priori, Construct F showed the best expression, more than 2.6-fold better than the mean expression of the other seven constructs. The superior performance of Construct F was due to the inclusion of a 5′ UTR comprising nucleotides of SEQ ID: 74 and a 3′ UTR comprising nucleotides of SEQ ID: 92.

This result was surprising, in part because SEQ ID: 92 is not a naturally-occurring UTR. SEQ ID: 92, which was included in Construct F, was expected to reduce, rather than increase, CAR expression.

### Example 7: Improved Polyadenine Tail for a CAR-Encoding mRNA Construct

A controlled experiment was conducted to test the addition of certain 3′ polyadenine tails to a CAR-encoding mRNA construct.

Numerous mRNA constructs were prepared that comprised the same 5′UTR, open reading frame and 3′UTR encoding an anti-BCMA CAR protein but included or did not include a 3′ polyadenine tail of 78 or more nucleotides. Each construct comprised exactly one mRNA sequence selected from: SEQ ID:46, SEQ ID:41, SEQ ID:42, SEQ ID:43, SEQ ID:44 or SEQ ID:45. In each construct the polyadenine tail was located 3′ with respect to the 3′ UTR, as such 3′ UTR is described in Example 2. Additional polyadenylation was performed enzymatically on an mRNA construct that comprised the nucleotide sequence of SEQ ID: 36 to obtain an otherwise identical mRNA construct wherein the polyadenine tail comprised over 150 nucleotides. For each of the mRNA constructs, CAR T cells were prepared by transfection into human CD8+ cells, substantially as described in Example 1. About 24 hours after transfection, the CAR T cells were tested for level of CAR expression substantially as described in Example 1. Results from the two experiments are shown below in Tables 13 and 14.

TABLE 13
Relative expression of full-length CARs having a 78-mer PolyA
tail as compared to CARs having no PolyA tail. Fold improvement
of expression is provided for each CAR construct.

| | Relative Expression* | | |
|---|---|---|---|
| Construct | Without PolyA | 78-mer PolyA | Fold Improvement |
| SEQ ID: 46 | 100 | 578 | 5.8 |
| SEQ ID: 41 | 99 | 310 | 3.1 |
| SEQ ID: 42 | 50 | 210 | 4.2 |
| SEQ ID: 43 | 105 | 222 | 2.1 |
| SEQ ID: 44 | 106 | 335 | 3.2 |
| SEQ ID: 45 | 57 | 425 | 7.5 |
| Mean | 86 | 347 | 4.3 (p < 0.001) |

*Mean fluorescence intensity, normalized to SEQ ID: 46 without PolyA = 100.

TABLE 14
Fold improvement in expression of the CAR of SEQ ID NO
36 having a 78-mer PolyA tail or a PolyA tail that has
more than 150 A residues. Fold improvement of expression
is provided for the CAR construct of SEQ ID NO: 36.

| | Relative Expression* | | |
|---|---|---|---|
| Construct | 78-mer PolyA | >150-mer PolyA | Fold Improvement |
| SEQ ID: 36 | 100 | 243 | 2.4 |

*Mean fluorescence intensity, normalized to SEQ ID: 46 without PolyA = 100.
*Mean fluorescence intensity, normalized to SEQ ID: 36 with 78-mer PolyA = 100.

Thus, direct experimental comparison showed that inclusion of a 78-mer polyadenine tail in the mRNA construct improved expression of inventive CAR proteins by a factor of 4.3 (p<0.001). Compared the 78-mer, inclusion of a >150-mer polyadenine tail further improved CAR expression by a factor of 2.4.

### Example 8: Synonymous Nucleotide Substitutions in a CAR-Encoding mRNA Construct

A controlled experiment was conducted to test whether certain synonymous nucleotide substitutions to the ORF of CAR-encoding mRNA construct the function of anti-BCMA CAR T cells.

Multiple mRNA constructs were prepared wherein ORFs encoded the same CAR protein of SEQ ID: 26, but wherein the nucleotide sequences of those ORFs differed, i.e., by the introduction of synonymous nucleotide substitutions. A construct comprising the ORF nucleotide sequence of SEQ ID: 116 served as the control. For each of the mRNA constructs, CAR T cells were prepared by transfection of mRNA into human CD8+ T cells, as described in Example 1. About 24 hours after transfection, the CAR T cells were tested for BCMA binding, as described in Example 1.

As compared to the construct of SEQ ID: 116, the constructs of SEQ ID: 122 (II new codon optimization) and SEQ ID: 123 (II dU) yielded CAR T cells for which BCMA binding was 1.65- and 3.43-fold higher, respectively. This was surprising, because the synonymous nucleotide substitutions did not alter the CAR protein sequences. In conclusion, certain synonymous nucleotide substitutions in the ORF of a CAR-encoding mRNA construct improved the BCMA binding properties of CAR T cells.

### Example 9: Test of CAR Specificity and Cross-Reactivity

CAR T cells produced from inventive mRNA constructs comprising the nucleotide sequences of SEQ ID: 31 were tested for CAR interactions with a cell microarray library that included 5528 human plasma membrane proteins and cell-surface-tethered human secreted proteins (Retrogenix, High Peak, UK). Otherwise comparable CAR T cells, wherein the mRNA constructs were of SEQ ID: 35 or SEQ ID: 40, were tested on a smaller subset of protein for confirmatory purposes. Otherwise comparable T cells that lacked the CAR were used to test for non-CAR-specific interactions. Each of the CARs of SEQ ID: 31, SEQ ID: 35, and SEQ ID: 40 specifically interacted with human BCMA but no interaction between the CAR and any other protein was observed. In conclusion, three inventive CARs specifically bound to BCMA but did not cross-react with another antigen.

### Example 10: In Vivo Assessment of CAR T Cell Antitumor Activity

CAR T cells produced from inventive mRNA constructs comprising the nucleotide sequences of SEQ ID: 30, SEQ ID: 31, and SEQ ID: 35 were tested in an animal model that measures the growth of human myeloma tumor cells in the animal. These mRNA constructs encoded CAR proteins that comprised, respectively, the sequences of SEQ ID: 20, SEQ ID: 21, and SEQ ID: 25.

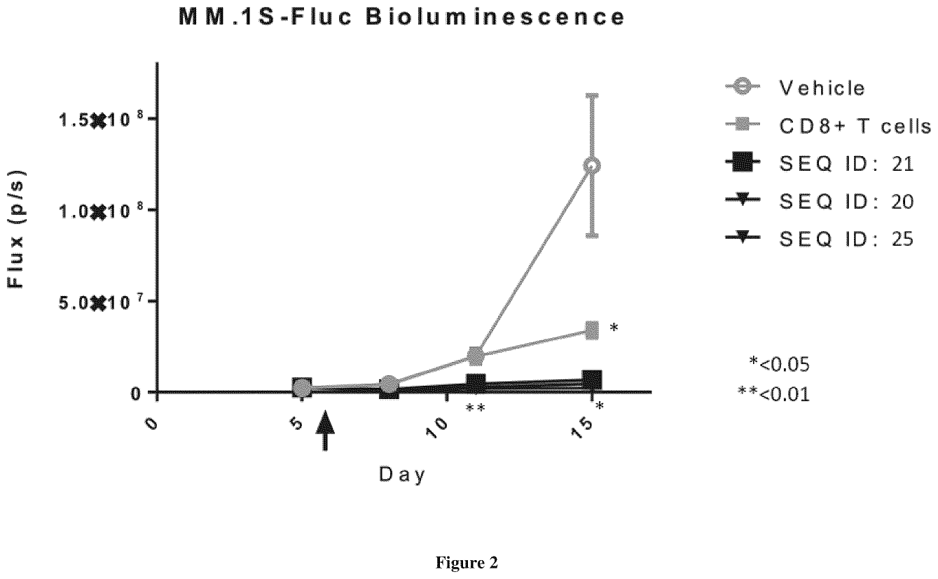

CAR T cells were prepared by transfection of the mRNA CAR construct into human CD8+ cells, as described in Example 1. Negative controls used in this study included (1) unmodified CD8+ cells and (2) vehicle only. NOD-scid-gamma (NSG) mice were inoculated with 2 million MM.1S-fluc human myeloma tumor cells. Tumor growth was monitored by serial bioluminescence imaging. On Day 6 mice were randomized to receive the vehicle, unmodified CD8+ T Cells, or CAR T Cells transfected to express a CAR of SEQ ID: 20, SEQ ID: 21, or SEQ ID: 25. FIG. 2 shows the results of tumor measurements. By Day 11, tumors grew extensively in mice treated with vehicle or modified CD8+ cells. In contrast, CAR T Cells transfected to express a CAR of SEQ ID: 20 (p=0.006), SEQ ID: 21 (p=0.01), or SEQ ID: 25 (p=0.004) significantly inhibited tumor growth. In conclusion, CAR T Cells prepared from three examples of the inventive, anti-BCMA CAR-encoding mRNA constructs significantly inhibited growth of human myeloma tumors in an animal model.

### Example 11: Effect of mRNA on CAR T Cell Survival

A controlled experiment was conducted to assess the effect of CAR-encoding mRNA on the survival of CAR T cells produced from cells donated by two myeloma patients. Two lots of CAR T cells, each lot produced from cells from two myeloma patients and by use of an inventive mRNA construct comprising the nucleotide sequences of SEQ ID: 31, were compared to two corresponding lots of CAR T cells, each lot produced from cells from the same two myeloma patients but by use of the reference mRNA construct comprising the nucleotide sequences of SEQ ID: 29. Except as noted here, each lot of CAR T cells was produced substantially by the methods described in Example 1. Cells were cultured for 21 hours after mRNA electroporation, whereupon cells were harvested, and viability was assessed. Results were as follows:

| | | Viability (%) |
|---|---|---|
| SEQ ID: 31 | Patient 1 | 90 |
| | Patient 2 | 81 |
| SEQ ID: 29 | Patient 1 | 83 |
| | Patient 2 | 67 |

The viability of CAR T cells of SEQ ID:29 was substantially lower than that of CAR T cells of SEQ ID:31. In conclusion, an inventive mRNA construct of SEQ ID:31 conferred better viability upon CAR T cells than a comparator mRNA construct of SEQ ID:29.

### Example 12: Use of Sleeping Beauty Transposon System to Make Permanently Modified CAR T Cells Expressing an Inventive Chimeric Antigen Receptor

The Sleeping Beauty transposon system was used to make permanently modified CAR T Cells that expressed an inventive Chimeric Antigen Receptor.

A first plasmid was constructed comprising the elements of an EF1a promoter, an IgG 5′UTR, an open reading frame encoding the amino acid sequence of SEQ ID:21, and a polyA tail, wherein the foregoing elements were collectively flanked by the Inverted Terminal Repeats of the Sleeping Beauty transposon (the “Seq-21 Transposon Plasmid”). A second plasmid was constructed comprising an EF1a promoter, an IgG 5′UTR, a Kozak consensus sequence, an open reading frame encoding SB11, and a poly A tail (the “SB11 plasmid”)

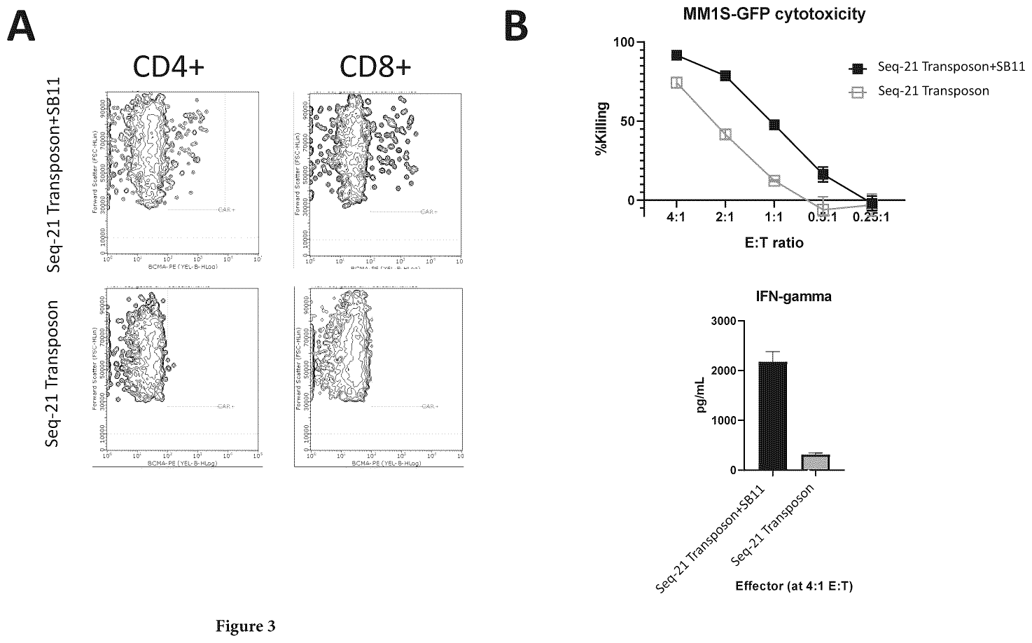

Peripheral Blood Mononuclear cells from a normal human donor were washed and resuspended in P3 buffer (Lonza) in the presence of Seq-21 Transposon plasmid alone or in the presence of both the Seq-21 Transposon plasmid and the SB11 plasmid. Cells were electroporated (4D Nucleofector®, Lonza) to introduce the plasmid(s) into cells. The cells were then transferred to culture for 5 hours. Cells were transferred to a stimulation culture comprising CD3/CD28 Dynabeads® (ThermoFisher). During expansion CD4+ and CD8+ T cells were analyzed for expression of the CAR by staining with BCMA-PE reagent and flow cytometry as described in Example 1. By day 14, T cells had expanded by 30-fold. Cells modified with both the SB11 and Seq-21 Transposon plasmids showed 5-10% of cells reactive for BCMA-PE (FIG. 3A). Cells expanded after transfection with the Seq-21 Transposon Plasmid alone showed no reactivity. Functional CAR T cell activity was evaluated using cytotoxicity assays with the multiple myeloma target cell line MM1S-GFP as described in Example 1 using multiple Effector:Target ratios using cell numbers based on total viable cells. Cytokine (interferon-γ) production was also evaluated on coculture supernatants from the 4:1 Effector:Target ratio by ELISA. FIG. 3B shows data from Day 9 cells illustrating specific cytotoxicity and interferon-γ production conferred by permanent modification with the anti-BCMA CAR delivered with the Seq-21 Transposon Plasmid and SB11 Plasmid, but not conferred by the Seq-21 Transposon Plasmid alone.

Thus, the Sleeping Beauty transposon system was successfully used to produce CAR T Cells that expressed an inventive anti-BCMA CAR. These cells demonstrated anti-BCMA cytotoxicity and cytokine secretion. They are expected to express the CAR permanently.

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which two or more members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

1-133. (canceled)

134. A nucleic acid encoding a protein comprising an antigen-binding portion, wherein the antigen binding portion is an anti-B-Cell Maturation Antigen (BCMA) binding domain comprising:
a heavy chain complementarity determining region 1 (CDRH1) comprising any one of SEQ ID NOs: 1-3;
a heavy chain complementarity determining region 2 (CDRH2) comprising any one of SEQ ID NOs: 4-6;
a heavy chain complementarity determining region 3 (CDRH3) comprising any one of SEQ ID NOs: 7-9;
a light chain complementarity determining region 1 (CDRL1) comprising any one of SEQ ID NOs: 10-11;
a light chain complementarity determining region 2 (CDRL2) comprising any one of SEQ ID NOs: 12-15; and
a light chain complementarity determining region 3 (CDRL3) comprising any one of SEQ ID NOs: 16-18;
provided that the anti-BCMA binding domain does not comprise all six of the following:
a CDRH1 comprising SEQ ID NO: 1;
a CDRH2 comprising SEQ ID NO: 4;
a CDRH3 comprising SEQ ID NO: 7;
a CDRL1 comprising SEQ ID NO: 10;
a CDRL2 comprising SEQ ID NO: 12; and
a CDRL3 comprising SEQ ID NO: 16.

135. The nucleic acid of claim 134, wherein the nucleic acid is mRNA.

136. The mRNA of claim 135, wherein the CDRH1 comprises SEQ ID NO: 1.

137. The mRNA of claim 135, wherein the CDRH1 comprises SEQ ID NO: 2.

138. The mRNA of claim 135, wherein the CDRH1 comprises SEQ ID NO: 3.

139. The mRNA of claim 135, wherein the CDRH2 comprises SEQ ID NO: 4.

140. The mRNA of claim 135, wherein the CDRH2 comprises SEQ ID NO: 5.

141. The mRNA of claim 135, wherein the CDRH2 comprises SEQ ID NO: 6.

142. The mRNA of claim 135, wherein the CDRH3 comprises SEQ ID NO: 7.

143. The mRNA of claim 135, wherein the CDRH3 comprises SEQ ID NO: 8.

144. The mRNA of claim 135, wherein the CDRH3 comprises SEQ ID NO: 9.

145. The mRNA of claim 135, wherein the CDRL1 comprises SEQ ID NO: 10.

146. The mRNA of claim 135, wherein the CDRL1 comprises SEQ ID NO: 11.

147. The mRNA of claim 135, wherein the CDRL2 comprises SEQ ID NO: 12.

148. The mRNA of claim 135, wherein the CDRL2 comprises SEQ ID NO: 13.

149. The mRNA of claim 135, wherein the CDRL2 comprises SEQ ID NO: 14.

150. The mRNA of claim 135, wherein the CDRL2 comprises SEQ ID NO: 15.

151. The mRNA of claim 135, wherein the CDRL2 comprises SEQ ID NO: 16.

152. The mRNA of claim 135, wherein the CDRL2 comprises SEQ ID NO: 17.

153. The mRNA of claim 135, wherein the CDRL2 comprises SEQ ID NO: 18.

154. The mRNA of claim 135, wherein the CDRH1 comprises SEQ ID NO: 1; the CDRH2 comprises SEQ ID NO: 4; the CDRH3 comprises SEQ ID NO: 8; the CDRL1 comprises SEQ ID NO: 10; the CDRL2 comprises SEQ ID NO: 15; and the CDRL3 comprises SEQ ID NO: 17.

155. The mRNA of claim 135, wherein the CDRH1 comprises SEQ ID NO: 2; the CDRH2 comprises SEQ ID NO: 6; the CDRH3 comprises SEQ ID NO: 9; the CDRL1 comprises SEQ ID NO: 11; the CDRL2 comprises SEQ ID NO: 14; and the CDRL3 comprises SEQ ID NO: 17.

156. The mRNA of claim 135, wherein the CDRH1 comprises SEQ ID NO: 2; the CDRH2 comprises SEQ ID NO: 6; the CDRH3 comprises SEQ ID NO: 9; the CDRL1 comprises SEQ ID NO: 11; the CDRL2 comprises SEQ ID NO: 15; and the CDRL3 comprises SEQ ID NO: 17.


SEQUENCE LISTING (133 sequences)

SEQ ID NO: 1 - protein, 5 aa, synthetic construct
  REGION 1..5: note = Synthetic Polypeptide
DYSIN

SEQ ID NO: 2 - protein, 5 aa, synthetic construct
  REGION 1..5: note = Synthetic Polypeptide
SYSIN

SEQ ID NO: 3 - protein, 5 aa, synthetic construct
  REGION 1..5: note = Synthetic Polypeptide
SYDIN

SEQ ID NO: 4 - protein, 17 aa, synthetic construct
  REGION 1..17: note = Synthetic Polypeptide
WINTETREPAYAYDFRG

SEQ ID NO: 5 - protein, 17 aa, synthetic construct
  REGION 1..17: note = Synthetic Polypeptide
WINTNTGNPTYAQGFTG

SEQ ID NO: 6 - protein, 17 aa, synthetic construct
  REGION 1..17: note = Synthetic Polypeptide
WINTETREPAYAQGFTG

SEQ ID NO: 7 - protein, 8 aa, synthetic construct
  REGION 1..8: note = Synthetic Polypeptide
DYSYAMDY

SEQ ID NO: 8 - protein, 8 aa, synthetic construct
  REGION 1..8: note = Synthetic Polypeptide
DYTYGMDY

SEQ ID NO: 9 - protein, 8 aa, synthetic construct
  REGION 1..8: note = Synthetic Polypeptide
DYLYSLDF

SEQ ID NO: 10 - protein, 15 aa, synthetic construct
  REGION 1..15: note = Synthetic Polypeptide
RASESVTILGSHLIH

SEQ ID NO: 11 - protein, 15 aa, synthetic construct
  REGION 1..15: note = Synthetic Polypeptide
RASESVSFLGINLIH

SEQ ID NO: 12 - protein, 8 aa, synthetic construct
  REGION 1..8: note = Synthetic Polypeptide
QLASNVQT

SEQ ID NO: 13 - protein, 8 aa, synthetic construct
  REGION 1..8: note = Synthetic Polypeptide
YLASNLET

SEQ ID NO: 14 - protein, 8 aa, synthetic construct
  REGION 1..8: note = Synthetic Polypeptide
YSASNLQS

SEQ ID NO: 15 - protein, 8 aa, synthetic construct
  REGION 1..8: note = Synthetic Polypeptide
NLASNVNT

SEQ ID NO: 16 - protein, 9 aa, synthetic construct
  REGION 1..9: note = Synthetic Polypeptide
LQSRTIPRT

SEQ ID NO: 17 - protein, 9 aa, synthetic construct
  REGION 1..9: note = Synthetic Polypeptide
LQSRTLPRT

SEQ ID NO: 18 - protein, 9 aa, synthetic construct
  REGION 1..9: note = Synthetic Polypeptide
LQSKNFPRT

SEQ ID NO: 19 - protein, 503 aa, synthetic construct
  REGION 1..503: note = Synthetic Polypeptide
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 462 aa, synthetic construct
  REGION 1..462: note = Synthetic Polypeptide
MALPVTALLLPLALLLHAARPDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 504 aa, synthetic construct
  REGION 1..504: note = Synthetic Polypeptide
MALPVTALLLPLALLLHAARPDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 462 aa, synthetic construct
  REGION 1..462: note = Synthetic Polypeptide
MALPVTALLLPLALLLHAARPDIVLTQSPASLAVSPGQRATITCRASESVSFLGINLIHWYQQKPGQPPKLLIYSASNLQSGVPARFSGSGSGTDFTLTISSVEPEDTANYYCLQSKNFPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 504 aa, synthetic construct
  REGION 1..504: note = Synthetic Polypeptide
MALPVTALLLPLALLLHAARPDIVLTQSPASLAVSPGQRATITCRASESVSFLGINLIHWYQQKPGQPPKLLIYSASNLQSGVPARFSGSGSGTDFTLTISSVEPEDTANYYCLQSKNFPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 462 aa, synthetic construct
  REGION 1..462: note = Synthetic Polypeptide
MALPVTALLLPLALLLHAARPDIVLTQSPASLAVSPGQRATITCRASESVTILGSHLIHWYQQKPGQPPKLLINLASNVNTGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTDYSINWVRQAPGKGLEWVGWINTETREPAYAYDFTGRFTFSADTSKSMAYLQINSLRAEDTAVYYCALDYTYGMDYWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 504 aa, synthetic construct
  REGION 1..504: note = Synthetic Polypeptide
MALPVTALLLPLALLLHAARPDIVLTQSPASLAVSPGQRATITCRASESVTILGSHLIHWYQQKPGQPPKLLINLASNVNTGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTDYSINWVRQAPGKGLEWVGWINTETREPAYAYDFTGRFTFSADTSKSMAYLQINSLRAEDTAVYYCALDYTYGMDYWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 462 aa, synthetic construct
  REGION 1..462: note = Synthetic Polypeptide
MALPVTALLLPLALLLHAARPDIVLTQSPSSLSASVGDRATISCRASESVTILGSHLIHWYQQKPGQAPKLLIQLASNVQTGVPARFSGSGSGTDFTLTISSVEPEDVAVYYCLQSRTIPRTFGQGTKVEIKGSTSGSGKPGSGEGSTKGQIQLVQSGPELKKPGGSVKISCAASGYTFTDYSINWVRQAPGKGLEWVGWINTETREPAYAYDFRGRFTFSADTSKSTAYLQMNSLRAEDTAVYYCALDYSYAMDYWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 462 aa, synthetic construct; identical to SEQ ID NO: 26
  REGION 1..462: note = Synthetic Polypeptide

SEQ ID NO: 28 - protein, 462 aa, synthetic construct
  REGION 1..462: note = Synthetic Polypeptide
MALPVTALLLPLALLLHAARPDIVLTQSPASLAVSPGQRATITCRASESVTILGSHLIHWYQQKPGQPPKLLIQLASNVQTGVPARFSGSGSGTDFTLTISSVEPEDTANYYCLQSRTIPRTFGQGTKVEIKGSTSGSGKPGSGEGSTKGQIQLVQSGPELKKPGGSVKISCKASGYTFTDYSINWVRQAPGKGLEWVGWINTETREPAYAYDFTGRFTFSADTSKSMAYLQINSLRAEDTAVYYCALDYSYAMDYWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - DNA, 1753 bp, synthetic construct
  misc_feature 1..1753: note = Synthetic Polynucleotide
  source 1..1753: mol_type = other DNA
aggaaataagagagaaaagaagagtaagaagaaatataagagccaccatggccctgcccgtgacagctctgctgctccctctggctctgctgctgcacgctgccagacccgacatcgtgctgacacaaagcccccctagcctggccatgagcctgggcaagagggccacaatctcctgcagggccagcgagtccgtgaccatcctgggcagccacctgatccactggtaccagcagaagcctggccagccccctaccctgctgatccagctggcctccaacgtgcagacaggagtgcccgctaggttctccggctccggcagcagaaccgactttaccctgaccatcgaccccgtcgaggaggacgacgtggctgtctactattgcctgcagagcaggaccatccccaggaccttcggaggcggcacaaagctggagatcaagggcagcaccagcggctccggaaagcctggcagcggagaaggctccaccaagggacagattcagctggtgcagagcggacccgagctgaagaagcccggcgaaacagtcaagatcagctgcaaagcttccggctacaccttcacagactacagcatcaactgggtgaaaagggcccccggcaagggcctgaaatggatgggctggatcaacaccgagaccagggagcccgcctacgcctacgactttaggggcaggttcgccttcagcctggagacatccgctagcaccgcttacctgcaaattaataacctgaagtacgaggacaccgccacctatttttgtgccctggactacagctacgcaatggactattggggacagggaacctccgtgaccgtgagcagctttgtgcccgtgttcctgcccgccaaacccaccaccacacctgctcctaggccccctacacctgcccctaccatcgcttcccagcccctgagcctcagacctgaagcctgcagacctgctgctggaggcgccgtccacacaaggggactggacttcgcctgtgacatctacatctgggctcctctcgccggcacctgtggagtgctgctgctgtccctggtcatcaccctgtactgcaaccacaggaacaggagcaagaggtccaggctgctgcatagcgactacatgaacatgacccccagaaggcccggacccaccaggaagcactaccagccctacgccccccctagggattttgctgcctacaggtccagagtgaagttcagcagatccgctgatgcccctgcctatcagcagggccagaaccaactctacaacgagctgaacctgggaaggagggaggagtacgacgtgctcgacaagagaaggggaagggatcctgaaatgggcggcaagcccaggagaaagaaccctcaggagggcctgtacaacgaactgcagaaggataagatggccgaggcctacagcgaaatcggcatgaaaggcgagagaaggagaggcaagggccacgacggactgtaccagggcctgtccaccgccaccaaagacacctacgatgccctgcacatgcaggccctgccccccaggtgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 30 - DNA, 1660 bp, synthetic construct
  misc_feature 1..1660: note = Synthetic Polynucleotide
  source 1..1660: mol_type = other DNA
agacccaagctggctagctctaaagaagcccctgggagcacagctcatcaccatggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgtccttcctgggcatcaacctgatccactggtaccagcagaagcccggacagccacccaaactgctgatctactccgccagcaacctgcagtcaggcgtgccagccagattcagcggcagcggcagcggaaccgacttcaccctgacaatcagctcagtggaaccagaagacaccgccaactactactgcctgcagagcagaaccctgcccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcacctcctacagcatcaactgggtgcgccaggccccaggcaagggcctggaatgggtcggctggatcaacaccgagacaagagagcccgcctacgcccagggcttcacaggcagattcaccttcagcgcagacacaagcaagagcatggcctacctgcagatcaactccctgagagcagaggacaccgccgtctactactgcgccctggactacctctactccctggacttctggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacagagtgaagttcagcagatccgccgacgcaccagcctaccagcagggacagaaccagctgtacaacgagctgaacctggggagaagagaagagtacgacgtgctggataagcggagaggcagagaccctgagatgggcggcaagcccagacggaagaacccacaagagggcctgtacaacgagctgcagaaagacaagatggccgaggcctacagcgagatcggaatgaagggcgagcgcagaagaggcaagggacacgacggactgtaccagggcctgagcacagccaccaaggatacctacgatgccctgcacatgcaggccctgccaccaagatgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagca

SEQ ID NO: 31 - DNA, 1786 bp, synthetic construct
  misc_feature 1..1786: note = Synthetic Polynucleotide
  source 1..1786: mol_type = other DNA
agacccaagctggctagctctaaagaagcccctgggagcacagctcatcaccatggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgtccttcctgggcatcaacctgatccactggtaccagcagaagcccggacagccacccaaactgctgatctactccgccagcaacctgcagtcaggcgtgccagccagattcagcggcagcggcagcggaaccgacttcaccctgacaatcagctcagtggaaccagaagacaccgccaactactactgcctgcagagcagaaccctgcccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcacctcctacagcatcaactgggtgcgccaggccccaggcaagggcctggaatgggtcggctggatcaacaccgagacaagagagcccgcctacgcccagggcttcacaggcagattcaccttcagcgcagacacaagcaagagcatggcctacctgcagatcaactccctgagagcagaggacaccgccgtctactactgcgccctggactacctctactccctggacttctggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacaaacgcgggagaaaaaagctcctctacatcttcaagcaaccattcatgcgcccagtccaaaccacacaggaggaagacggatgcagctgcaggttccccgaagaggaggaaggcggatgcgagctcagagtgaagttcagcagatccgccgacgcaccagcctaccagcagggacagaaccagctgtacaacgagctgaacctggggagaagagaagagtacgacgtgctggataagcggagaggcagagaccctgagatgggcggcaagcccagacggaagaacccacaagagggcctgtacaacgagctgcagaaagacaagatggccgaggcctacagcgagatcggaatgaagggcgagcgcagaagaggcaagggacacgacggactgtaccagggcctgagcacagccaccaaggatacctacgatgccctgcacatgcaggccctgccaccaagatgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagca

SEQ ID NO: 32 - DNA, 1660 bp, synthetic construct
  misc_feature 1..1660: note = Synthetic Polynucleotide
  source 1..1660: mol_type = other DNA
agacccaagctggctagctctaaagaagcccctgggagcacagctcatcaccatggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgtccttcctgggcatcaacctgatccactggtaccagcagaagcccggacagccacccaaactgctgatctactccgccagcaacctgcagtcaggcgtgccagccagattcagcggcagcggcagcggaaccgacttcaccctgacaatcagctcagtggaaccagaagacaccgccaactactactgcctgcagagcaagaacttccccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcacctcctacagcatcaactgggtgcgccaggccccaggcaagggcctggaatgggtcggctggatcaacaccgagacaagagagcccgcctacgcccagggcttcacaggcagattcaccttcagcgcagacacaagcaagagcatggcctacctgcagatcaactccctgagagcagaggacaccgccgtctactactgcgccctggactacctctactccctggacttctggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacagagtgaagttcagcagatccgccgacgcaccagcctaccagcagggacagaaccagctgtacaacgagctgaacctggggagaagagaagagtacgacgtgctggataagcggagaggcagagaccctgagatgggcggcaagcccagacggaagaacccacaagagggcctgtacaacgagctgcagaaagacaagatggccgaggcctacagcgagatcggaatgaagggcgagcgcagaagaggcaagggacacgacggactgtaccagggcctgagcacagccaccaaggatacctacgatgccctgcacatgcaggccctgccaccaagatgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagca

SEQ ID NO: 33 - DNA, 1786 bp, synthetic construct
  misc_feature 1..1786: note = Synthetic Polynucleotide
  source 1..1786: mol_type = other DNA
agacccaagctggctagctctaaagaagcccctgggagcacagctcatcaccatggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgtccttcctgggcatcaacctgatccactggtaccagcagaagcccggacagccacccaaactgctgatctactccgccagcaacctgcagtcaggcgtgccagccagattcagcggcagcggcagcggaaccgacttcaccctgacaatcagctcagtggaaccagaagacaccgccaactactactgcctgcagagcaagaacttccccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcacctcctacagcatcaactgggtgcgccaggccccaggcaagggcctggaatgggtcggctggatcaacaccgagacaagagagcccgcctacgcccagggcttcacaggcagattcaccttcagcgcagacacaagcaagagcatggcctacctgcagatcaactccctgagagcagaggacaccgccgtctactactgcgccctggactacctctactccctggacttctggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacaaacgcgggagaaaaaagctcctctacatcttcaagcaaccattcatgcgcccagtccaaaccacacaggaggaagacggatgcagctgcaggttccccgaagaggaggaaggcggatgcgagctcagagtgaagttcagcagatccgccgacgcaccagcctaccagcagggacagaaccagctgtacaacgagctgaacctggggagaagagaagagtacgacgtgctggataagcggagaggcagagaccctgagatgggcggcaagcccagacggaagaacccacaagagggcctgtacaacgagctgcagaaagacaagatggccgaggcctacagcgagatcggaatgaagggcgagcgcagaagaggcaagggacacgacggactgtaccagggcctgagcacagccaccaaggatacctacgatgccctgcacatgcaggccctgccaccaagatgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagca

SEQ ID NO: 34 - DNA, 1660 bp, synthetic construct
  misc_feature 1..1660: note = Synthetic Polynucleotide
  source 1..1660: mol_type = other DNA
agacccaagctggctagctctaaagaagcccctgggagcacagctcatcaccatggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgacaatcctgggctcccacctgatccactggtaccagcagaagcccggacagccacccaaactgctgatcaacctggccagcaacgtgaacacaggcgtgccagccagattcagcggcagcggcagcggaaccgacttcaccctgacaatcagctcagtggaaccagaagacaccgccaactactactgcctgcagagcagaacactgcccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcaccgactacagcatcaactgggtgcgccaggccccaggcaagggcctggaatgggtcggctggatcaacaccgagacaagagagcccgcctacgcctacgacttcacaggcagattcaccttcagcgcagacacaagcaagagcatggcctacctgcagatcaactccctgagagcagaggacaccgccgtctactactgcgccctggactacacctacggcatggactactggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacagagtgaagttcagcagatccgccgacgcaccagcctaccagcagggacagaaccagctgtacaacgagctgaacctggggagaagagaagagtacgacgtgctggataagcggagaggcagagaccctgagatgggcggcaagcccagacggaagaacccacaagagggcctgtacaacgagctgcagaaagacaagatggccgaggcctacagcgagatcggaatgaagggcgagcgcagaagaggcaagggacacgacggactgtaccagggcctgagcacagccaccaaggatacctacgatgccctgcacatgcaggccctgccaccaagatgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagca

SEQ ID NO: 35 - DNA, 1786 bp, synthetic construct
  misc_feature 1..1786: note = Synthetic Polynucleotide
  source 1..1786: mol_type = other DNA
agacccaagctggctagctctaaagaagcccctgggagcacagctcatcaccatggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgacaatcctgggctcccacctgatccactggtaccagcagaagcccggacagccacccaaactgctgatcaacctggccagcaacgtgaacacaggcgtgccagccagattcagcggcagcggcagcggaaccgacttcaccctgacaatcagctcagtggaaccagaagacaccgccaactactactgcctgcagagcagaacactgcccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcaccgactacagcatcaactgggtgcgccaggccccaggcaagggcctggaatgggtcggctggatcaacaccgagacaagagagcccgcctacgcctacgacttcacaggcagattcaccttcagcgcagacacaagcaagagcatggcctacctgcagatcaactccctgagagcagaggacaccgccgtctactactgcgccctggactacacctacggcatggactactggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacaaacgcgggagaaaaaagctcctctacatcttcaagcaaccattcatgcgcccagtccaaaccacacaggaggaagacggatgcagctgcaggttccccgaagaggaggaaggcggatgcgagctcagagtgaagttcagcagatccgccgacgcaccagcctaccagcagggacagaaccagctgtacaacgagctgaacctggggagaagagaagagtacgacgtgctggataagcggagaggcagagaccctgagatgggcggcaagcccagacggaagaacccacaagagggcctgtacaacgagctgcagaaagacaagatggccgaggcctacagcgagatcggaatgaagggcgagcgcagaagaggcaagggacacgacggactgtaccagggcctgagcacagccaccaaggatacctacgatgccctgcacatgcaggccctgccaccaagatgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagca

SEQ ID NO: 36 - DNA, 1739 bp, synthetic construct
  misc_feature 1..1739: note = Synthetic Polynucleotide
  source 1..1739: mol_type = other DNA
agacccaagctggctagctctaaagaagcccctgggagcacagctcatcaccatggctctgccggtaacagcgcttcttctcccgcttgccctcctcttgcacgctgctcggccagatattgtcctcacacagagcccgtcctcactttcagcgagcgtcggggaccgcgctaccatcagttgtcgagccagtgaaagcgtcactattctggggagccaccttatacattggtatcaacaaaagccaggccaggcccccaaactgctgattcagctcgcatctaatgttcagactggagtgcctgcaaggtttagcggtagtgggtcagggacagatttcaccttgaccattagctcagttgagcctgaagatgtggccgtttattactgcctccaaagccgcactatacctcggaccttcggccagggaacaaaggtggagattaagggtagtacctcaggtagtggcaagccagggagcggcgaggggtctacaaagggtcaaattcaattggtgcagtccggaccagaactgaaaaaaccgggcgggagcgtcaagatttcctgtgcagcatcaggctatacatttactgattacagtattaactgggtgagacaagcgcctggaaagggccttgagtgggtgggttggattaacacggagactagagaacccgcgtacgcctacgattttagaggccggttcactttctccgccgatacaagtaaatccacagcatatctccaaatgaatagccttagagcggaggataccgccgtgtattattgcgcgctcgactattcttacgcaatggactactgggggcagggtactctcgtcaccgtatcctcatttgttcccgtatttttgcccgccaagccgaccactactccggcccctcgacctccgacccctgctccgactattgcgagtcaaccattgagccttcggcctgaggcatgtagaccggcagctggtggcgcagtccatacacgagggcttgattttgcgtgcgacatctatatatgggcgccattggctgggacgtgcggcgtccttctcttgtcactcgtgataactctgtactgcaatcacagaaaccgcgtgaagttttccagaagcgcggacgcgccggcctatcaacaagggcagaatcagctgtacaacgaactcaatttggggcgaagggaggagtatgatgttttggacaaaaggcgaggccgcgaccctgaaatgggcggtaagccacgcagaaaaaacccccaagagggattgtacaatgaactgcaaaaagacaagatggctgaggcatacagtgagataggaatgaaaggtgaacggcgccggggaaaaggacatgacgggttgtaccagggtcttagcactgcaacgaaagatacctatgacgccctgcacatgcaagcattgcccccacggtgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 37 - DNA, 1529 bp, synthetic construct
  misc_feature 1..1529: note = Synthetic Polynucleotide
  source 1..1529: mol_type = other DNA
agacccaagctggctagctctaaagaagcccctgggagcacagctcatcaccatggcactgccggtaacagcgctcctcctcccgctcgccctcctcctccacgcagcacggccagacatcgtcctcacacagagcccgagcagcctcagcgcgagcgtcggggaccgcgcaaccatcagctgccgagccagcgaaagcgtcacaatcctcgggagccacctgatacactggtaccaacaaaagccaggccaggcccccaaactgctgatccagctcgcaagcaacgtccagacaggagtgccagcaaggttcagcggaagcgggagcggaacagacttcaccctcaccatcagcagcgtcgagccagaagatgtggccgtctactactgcctccaaagccgcacaataccacggaccttcggccagggaacaaaggtggagatcaagggaagcaccagcggaagcggcaagccagggagcggcgaggggagcacaaagggacaaatccaactcgtgcagagcggaccagaactgaaaaagccgggcggaagcgtcaagatcagctgcgcagcaagcggctacacattcacagactacagcatcaactgggtgagacaagcgccaggaaagggcctcgaatgggtgggatggatcaacacggagacaagagaacccgcatacgcctacgacttcagaggccggttcacattcagcgccgacacaagcaagagcacagcatacctccaaatgaacagcctcagagcggaggacaccgccgtgtactactgcgcgctcgactacagctacgcaatggactactggggacagggaacactcgtcaccgtaagcagcttcgtacccgtattcctccccgccaagccgaccacaacaccggccccacgaccaccaacaccagccccgacaatcgcgagccaaccactcagcctccggccagaggcgtgcagaccggcagcaggaggcgcagtccacacacgaggactcgacttcgcgtgcgacatatacatctgggcaccactcgcaggaacgtgcggcgtcctcctcctcagcctcgtgataacactgtactgcaaccacagaaaccgcgtgaagttcagcagaagcgcggacgcgccggcctaccaacaaggacagaaccagctgtacaacgaactcaacctcggacgaagggaggagtacgacgtcctcgacaaaaggcgaggccgcgacccagaaatgggcggaaagccacgcagaaaaaacccccaagagggactctacaacgaactgcaaaaggacaagatggcagaggcatacagcgagataggaatgaaaggagaacggcgccggggaaaaggacacgacgggctctaccagggactcagcacagcaacgaaagacacctacgacgccctgcacatgcaagcactccccccacggtgaacctgagactggtggcttctagaagcagccattaccaactgtaccttcccttcttgctcagccaataaatatatcctctttcactcag

SEQ ID NO: 38 - DNA, 1724 bp, synthetic construct
  misc_feature 1..1724: note = Synthetic Polynucleotide
  source 1..1724: mol_type = other DNA
gggagacccaagctggctagcctcgcagttcggcggtcccgcgggtctgtctgttgcttcaacagtgtttggacggaacagatccggggactctcttccagccatggcactgcctgttacagctctgctgctgcctctggcactgctgcttcatgccgccagacctgacattgttctgacacagtccccttctagtttgagcgcgagtgttggcgaccgagctacgataagctgtcgagctagcgaaagcgtgacgatcctgggttcccaccttattcattggtatcaacagaaaccagggcaggctcctaaacttctcatacagttggccagcaatgttcaaaccggtgtgcccgcacgcttcagcggatcagggtcaggaaccgatttcacgctcacaattagcagtgtggagcctgatgacgtagccgtgtactactgtctgcaaagccgcaccatcccgagaacgtttgggggcggcacgaaattggaaattaaaggaagcacttccgggagtgggaagcctgggagtggcgaagggtcaacaaaggggcagatacagttggtccagagcgggcccgagttgaaaaagccaggtggatcagttaaaatttcttgtgctgcgtccggatatacttttactgactattctatcaactgggtcaaacaagctcccggtaaaggattggaatgggtcggatggataaacactgaaacccgcgagcctgcttatgcttacgattttcgaggtagattcaccttctcacttgatacatccaaaagtacggcttacctgcaaatgaactcactgagagcagaagataccgccgtttactattgtgccctcgactatagttacgccatggattattggggacaagggactctcgtgaccgtaagtagctttgtgcctgtgttcctgcctgccaagcctaccacaacaccagctcctagacctccaactcctgctcctacaatcgcctctcagccactgtctctgaggcctgaagcttgtagacctgctgctggcggagccgtgcataccagaggactggatttcgcctgcgacatctacatctgggctcctctggctggaacatgtggcgtgctgctgctgagcctggtcatcaccctgtactgcaaccaccggaacagagtgaagttcagcagatccgccgatgctcctgcctaccagcagggacagaaccagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcacagccaccaaggatacctatgatgccctgcacatgcaggccctgcctccaagatgagggcccgtttaaacccgctggctcgctttcttgctgtccaatttctattaaaggttcctttgttccgtaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 39 - DNA, 1660 bp, synthetic construct
  misc_feature 1..1660: note = Synthetic Polynucleotide
  source 1..1660: mol_type = other DNA
agacccaagctggctagctctaaagaagcccctgggagcacagctcatcaccatggctctgccggtaacagcgcttcttctcccgcttgccctcctcttgcacgctgctcggccagacatagtattgacgcaatcccccgcctctctcgcagtctctcccggacaacgggccactataacttgcagggcaagtgagagtgtgacaatcctgggatcacacctgatacattggtatcagcaaaagccgggtcaaccccctaagttgctgatacaactggcatctaatgtacaaacgggagtccccgcacggttctctggttctggttctggcactgacttcacattgacaatctcaagtgttgaacctgaggacacagccaattattattgcttgcaatctcgaacaattcctcgcactttcggtcagggcacgaaggttgaaattaaaggtagtacctcaggtagtggcaagccagggagcggcgaggggtctacaaagggtcaaatccagcttgtgcaatctggcccggagctgaaaaaacccggtggatctgtgaaaattagctgcaaagcgtcaggatatacattcacagactatagcatcaactgggtccggcaggcacccggtaagggtctggaatgggttggttggattaacactgagacccgtgagcccgcctatgcatacgattttacgggacggtttactttcagcgccgacacgagcaagagcatggcgtacctccagattaacagtctgagggcggaggacacagccgtctactattgcgcgttggattatagctacgcgatggactactgggggcaaggtacacttgtcacggtgtcatcttttgttcccgtatttttgcccgccaagccgaccactactccggcccctcgacctccgacccctgctccgactattgcgagtcaaccattgagccttcggcctgaggcatgtagaccggcagctggtggcgcagtccatacacgagggcttgattttgcgtgcgacatctatatatgggcgccattggctgggacgtgcggcgtccttctcttgtcactcgtgataactctgtactgcaatcacagaaaccgcgtgaagttttccagaagcgcggacgcgccggcctatcaacaagggcagaatcagctgtacaacgaactcaatttggggcgaagggaggagtatgatgttttggacaaaaggcgaggccgcgaccctgaaatgggcggtaagccacgcagaaaaaacccccaagagggattgtacaatgaactgcaaaaagacaagatggctgaggcatacagtgagataggaatgaaaggtgaacggcgccggggaaaaggacatgacgggttgtaccagggtcttagcactgcaacgaaagatacctatgacgccctgcacatgcaagcattgcccccacggtgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagca

SEQ ID NO: 40 - DNA, 1660 bp, synthetic construct
  misc_feature 1..1660: note = Synthetic Polynucleotide
  source 1..1660: mol_type = other DNA
agacccaagctggctagctctaaagaagcccctgggagcacagctcatcaccatggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgacaatcctgggctcccacctgatccactggtaccagcagaagcccggacagccacccaaactgctgatccagctggccagcaacgtgcagacaggcgtgccagccagattcagcggcagcggcagcggaaccgacttcaccctgacaatcagctcagtggaaccagaagacaccgccaactactactgcctgcagagcagaacaatccccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcaccgactacagcatcaactgggtgcgccaggccccaggcaagggcctggaatgggtcggctggatcaacaccgagacaagagagcccgcctacgcctacgacttcacaggcagattcaccttcagcgcagacacaagcaagagcatggcctacctgcagatcaactccctgagagcagaggacaccgccgtctactactgcgccctggactacagctacgccatggactactggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacagagtgaagttcagcagatccgccgacgcaccagcctaccagcagggacagaaccagctgtacaacgagctgaacctggggagaagagaagagtacgacgtgctggataagcggagaggcagagaccctgagatgggcggcaagcccagacggaagaacccacaagagggcctgtacaacgagctgcagaaagacaagatggccgaggcctacagcgagatcggaatgaagggcgagcgcagaagaggcaagggacacgacggactgtaccagggcctgagcacagccaccaaggatacctacgatgccctgcacatgcaggccctgccaccaagatgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagca

SEQ ID NO: 41 - DNA, 1644 bp, synthetic construct
  misc_feature 1..1644: note = Synthetic Polynucleotide
  source 1..1644: mol_type = other DNA
gggagacccaagctggctagcctcgcagttcggcggtcccgcgggtctgtctgttgcttcaacagtgtttggacggaacagatccggggactctcttccagccatggctctgcccgtaaccgccctgctcctgccactcgcactcctcctgcatgcggctaggccggatattgtaatgacgcaatcccctgactcactgagtgtgtccttgggagagagagccacgataaattgtcgggcctccgagtcagtcactatactcggttctcacctgatccactggtaccaacaaaagcctgggcagccaccgaaacttttgatacagttggcctccaatgtccaaactggggttcccgaccgctttagcggaagcgggagcggtaccgattttaccctcaccatatcatcactccaagctgaggacgtggccgtttactactgtctccagtcaaggacaataccgcgaacattcggtggaggtactaaagttgaaataaaaggttccacaagtggctcagggaagccagggagtggtgagggaagcacaaaaggccaaatacaactggtgcagtctggagcagaagtcaagaagccaggtgcctccgttaagatttcttgtaaagcttccggctatacctttaccgattacagcattaattgggtccgccaggcccctgggcagggccttgagtggatgggctggataaatacagagactagagaacccgcgtatgcatacgattttcgaggccgggtgactatgacgcgcgacacctccgcgtctactgcatacctgcagatcagtagcctgaaagccgaagataccgccgtttacttctgcgccctcgactattcttacgcaatggactactggggacaaggttctctcgttactgtatctagctttgttccagtgttcctcccggcgaaacccaccactaccccagcccctcgcccaccgactccggcgccgacgattgccagtcagccgttgagcctccgaccggaggcgtgtcgccccgcggcaggaggggccgtgcatactaggggacttgattttgcctgtgacatatatatatgggcccctttggccggtacctgtggtgtcctgctgcttagtttggtgataacactctattgtaaccatcgcaatcgagttaagttcagtcggtcagctgatgctccggcttaccagcaaggccagaatcagctgtacaatgagctcaacctgggtcggcgagaagagtatgatgtactggacaaacggagaggtcgggatcccgaaatggggggtaaaccacgcaggaagaacccacaagaggggttgtacaatgaactgcagaaagataagatggccgaggcttacagtgaaatcgggatgaaaggagaacggagacgaggtaaggggcacgacggactctatcaaggactcagcaccgctacaaaggatacctatgatgcactccatatgcaagcccttcccccaaggtaggggcccgtttaaacccgctggctcgctttcttgctgtccaatttctattaaaggttcctttgttccgtaagtccaactactaaactgggggatattatgaagggccttgagcatctggattctgcctaataaaaaacatttattttcattgc

SEQ ID NO: 42 - DNA, 1644 bp, synthetic construct
  misc_feature 1..1644: note = Synthetic Polynucleotide
  source 1..1644: mol_type = other DNA
gggagacccaagctggctagcctcgcagttcggcggtcccgcgggtctgtctgttgcttcaacagtgtttggacggaacagatccggggactctcttccagccatggcgctcccagttacagccttgttgttgcccttggccctccttctccacgctgcacgcccggacatagttttgacccagagccctgcaagtttggctgtcagtcttggagagcgagctacaattagttgcagagcgtctgaaagcgtctccatattgggcagtcaccttttgcactggtaccaacagaagccaggacagccaccaaagctgcttatttacctcgcctccaatttgcaaactggagtaccggctcgattctccggatctggttctggaacagacttcacccttacgatatcaagtctggaggctgaggatgtcgcggtttactactgcctccaatcacgaacaataccgagaacgttcggccaggggactaaacttgaaatcaagggtagtacgagtggaagtgggaaacctgggtctggtgaggggtctacgaaagggcaaatacagctggttcaatcaggcgccgaagtaaaaaaaccgggggcatctgtgaaaatatcctgcaaagcgtctggatacacatttacagactattccatcaactgggtccgccaggcccccggccaaggtctggaatggatgggctggatcaacacagaaacccgggaacctgcatacgcttacgattttcgcggccgggtcactatgacccgcgatacaagtgcctccaccgcatatctccaaatctcatctttgaaggcagaagatacagccgtctacttttgtgcactggactatagctatgcgatggattactggggtcaaggcagccttgttactgtgtccagctttgtgcccgtatttctcccggccaagccaactactacccctgccccgcgaccgccaacgccagcgcctaccattgcgagtcaaccactctcactgcggccagaagcctgccgacctgccgccgggggggcagtacacactcgcggtcttgattttgcttgcgacatatatatctgggccccactggctgggacgtgtggcgttttgctgcttagtttggtcataaccctctattgcaaccacagaaatcgcgttaaatttagcagaagtgctgacgcacctgcataccagcaaggtcagaaccaactgtataatgaactgaatttgggacgcagggaagagtatgacgtcttggataaaagaagaggacgcgatccggagatgggtgggaaacctcgccgaaagaacccccaagaaggtttgtataatgaactccagaaggacaaaatggcggaggcgtatagcgagattggaatgaagggggaaagacgacgcggcaaaggtcatgacggattgtatcagggcctctctacggctactaaagatacttatgatgctctccatatgcaggcgttgccaccaaggtgagggcccgtttaaacccgctggctcgctttcttgctgtccaatttctattaaaggttcctttgttccgtaagtccaactactaaactgggggatattatgaagggccttgagcatctggattctgcctaataaaaaacatttattttcattgc

SEQ ID NO: 43 - DNA, 1644 bp, synthetic construct
  misc_feature 1..1644: note = Synthetic Polynucleotide
  source 1..1644: mol_type = other DNA
gggagacccaagctggctagcctcgcagttcggcggtcccgcgggtctgtctgttgcttcaacagtgtttggacggaacagatccggggactctcttccagccatggctctgcccgtaaccgcactgcttctgccactcgctctgttgctccacgccgctagacctgacatcgtgctgacgcaaagcccggcgtcactcgccgttagtttgggaaaacgggcgacaatatcttgtagagcttctgaaagcgtcactatcctgggttcacatctgatacactggtaccagcagaagccaggacagccacccaaactgctgatatatcttgcttctaatgtacaaacaggcgtaccagcccgcttcagcggttccgggtctggtacagattttactttgaccatatccagtttggaggctgaggacgttgccgtctactactgtctccaaagtcggactataccgaggacctttggtcaggggacaaagcttgagataaaagggtccacatccgggtcaggaaaacctggaagtggggagggatctacaaaagggcaggtacaactggtccagtccggggcggaagtaaaaaaacccggggcctctgtaaaaatttcctgtaaggcctctggttacacgtttaccgactacagtatcaattgggtgcggcaagccccgggccaagggttggaatggatgggttggataaatactgagacacgggaaccagcatacgcctacgattttcggggtcgagtcacaatgacgagagatacttctgccagtactgcttatctccagatcagttccctcaaagcggaagacaccgcagtatacttctgtgccctcgactattcatacgcaatggattattggggtcaaggctcacttgtgacggtgagttccttcgtccccgtcttcttgccggcgaagcccactactacgccggcaccacgcccccccactcctgcgccaactatagcttcacaacccttgagcctgaggcctgaggcgtgtaggccagcagcaggtggggcagtgcacactaggggcctcgattttgcctgcgatatctatatttgggcaccgctcgcgggtacgtgcggcgttcttttgttgtccttggtcatcacactgtactgtaaccatcggaatcgcgtaaaattcagccggagtgccgatgcaccggcgtaccagcaaggtcaaaaccagctctacaatgagctgaatctgggaagacgcgaagagtatgacgttcttgataagaggagaggccgagaccccgaaatgggggggaagcccaggcgaaagaatccccaggagggtttgtacaatgagcttcaaaaagacaagatggcagaggcctactccgaaatagggatgaaaggggaaagacggagaggcaagggtcatgacggactctatcagggtcttagcaccgccacgaaggacacatacgatgcccttcatatgcaagcactcccccctagatgagggcccgtttaaacccgctggctcgctttcttgctgtccaatttctattaaaggttcctttgttccgtaagtccaactactaaactgggggatattatgaagggccttgagcatctggattctgcctaataaaaaacatttattttcattgc

SEQ ID NO: 44 - DNA, 1644 bp, synthetic construct
  misc_feature 1..1644: note = Synthetic Polynucleotide
  source 1..1644: mol_type = other DNA
gggagacccaagctggctagcctcgcagttcggcggtcccgcgggtctgtctgttgcttcaacagtgtttggacggaacagatccggggactctcttccagccatggcgcttccagtcaccgctctccttttgccgctcgcactgcttctgcatgcggcccgccccgatatcgttatgacccagtctcctgacagtctgagtgtcagtctcggagagcgggcaacgattaattgtcgcgcctccgaaagcgttaccatactcgggagccatcttatccattggtaccaacaaaagccaggtcagcctcctaagttgttgattcaacttgctagcaacgtgcaaacgggcgttccagacaggtttagcggatccggaagcggaacagacttcactctgacgatatcaagtttgcaagcggaggacgtagcggtttactactgccttcagagtaggacaatccctagaacattcggcggcgggacaaaggtggaaataaagggatccaccagtggaagcgggaagccgggtagcggtgaaggcagcactaagggacaaatccaactcgtacagagtggggcagaggtgaagaaacctggggagagtgtaaaaatttcttgcaaagcctcaggctacacattcacggattatagcattaattgggtgcgacaggcgcccggtcaaggacttaagtggatgggctggatcaacacggaaacgcgcgaaccggcgtatgcctatgactttcgaggaaggttcgcattcagccttgacacgtccgcatcaaccgcctaccttcagatcagttcactcaaagctgaagatacggctgtgtatttttgcgcacttgattatagctatgccatggactactggggtcagggaaccttggttactgtatcttctttcgtaccagtttttttgccggctaaaccgactaccacgccggcaccacggcccccgacacccgctccaacaatagcgtcccaacccctttcacttaggccggaggcctgcagacctgctgctgggggagctgtccatactcggggacttgacttcgcgtgcgacatatacatctgggcgcctcttgccggcacctgtggtgtattgttgttgtctcttgttatcactctgtactgtaaccaccgcaacagggttaagttcagccgctccgcagatgctcctgcctaccagcagggacaaaatcaactttataacgaattgaaccttgggcgaagggaggaatatgatgttcttgacaaaaggcgcggcagggatccggagatgggaggtaagccacgcaggaagaatccgcaggagggcttgtataacgaactccagaaagataaaatggcagaagcctattccgagataggaatgaagggggagcgaagacggggaaaaggtcacgatgggctgtatcagggcctctctaccgccactaaggacacttatgacgcattgcacatgcaggcgttgccaccacggtgagggcccgtttaaacccgctggctcgctttcttgctgtccaatttctattaaaggttcctttgttccgtaagtccaactactaaactgggggatattatgaagggccttgagcatctggattctgcctaataaaaaacatttattttcattgc

SEQ ID NO: 45 - DNA, 1644 bp, synthetic construct
  misc_feature 1..1644: note = Synthetic Polynucleotide
  source 1..1644: mol_type = other DNA
gggagacccaagctggctagcctcgcagttcggcggtcccgcgggtctgtctgttgcttcaacagtgtttggacggaacagatccggggactctcttccagccatggcactcccagttacggctttgttgctcccgctcgcgttgttgctgcatgctgctcgcccagacatcgtcctcacgcaaagtcccgcaagtctcgccgtctcattgggcgaaagagcgacaatatcctgtcgagcgtcagagtctgttagtatccttggcagccactatctggcttggtatcagcagaaaccagggcagcctcccaagctgctgatttatctcgcgagcaatctccagacaggtgtcccagcacgcttcagtggatctggcagcggaacagattttacactcacgatttcttccctggaggctgaagatgtggcagtgtactattgtcaacagagcagaactatcccgagaacatttgggcagggcactaaacttgaaatcaaagggagtacgagtggtagtggaaaaccgggatccggcgaaggctccacaaaggggcaaatacagctggtccagtctggagctgaggtgaaaaagcccggtgagtctgtaaagatttcctgcaaggcaagtggttacacttttacggattactcaatcaattgggttcggcaagcacctggacagggtttgaagtggatgggatggataaatacagaaacaagggaacctgcctatgcctatgactttcggggtagattcgccttctcacttgacacaagtgccagtacagcctatcttcagatttcaagcctgaaggcggaagatacggccgtctatttttgtgcgcttgactatagttatgcgatggattattggggccaaggcacactggtcacagttagctcttttgtccctgttttcctcccagcaaaaccaacaactacacccgcgccgaggccgcccacccctgcccctaccatcgcctcacagccattgagcctccgccccgaggcatgcagaccggcagctggaggtgccgtacacactcgagggctcgacttcgcatgtgatatttatatctgggcgccattggcgggtacttgcggtgtgctgctccttagccttgtcataaccctctactgcaatcatcgcaatagggtcaaattttcaaggtcagcagatgctccggcgtatcagcaggggcaaaaccaactttacaatgagctcaacctgggtagacgcgaggagtacgacgtgctcgacaaaaggaggggtcgggatccggaaatggggggcaaaccccgcaggaagaacccacaggaaggactgtataatgagctccagaaagacaaaatggcagaggcatattctgagataggaatgaaaggcgagcggagaagaggtaaagggcacgacggactttatcagggtctttcaacagcgactaaggatacatatgacgcattgcacatgcaagctctcccacctagatgagggcccgtttaaacccgctggctcgctttcttgctgtccaatttctattaaaggttcctttgttccgtaagtccaactactaaactgggggatattatgaagggccttgagcatctggattctgcctaataaaaaacatttattttcattgc

SEQ ID NO: 46 - DNA, 1644 bp, synthetic construct
  misc_feature 1..1644: note = Synthetic Polynucleotide
  source 1..1644: mol_type = other DNA
gggagacccaagctggctagcctcgcagttcggcggtcccgcgggtctgtctgttgcttcaacagtgtttggacggaacagatccggggactctcttccagccatggcactgcctgttacagctctgctgctgcctctggcactgctgcttcatgccgccagacctgacattgttctgacacagtccccttctagtttgagcgcgagtgttggcgaccgagctacgataagctgtcgagctagcgaaagcgtgacgatcctgggttcccaccttattcattggtatcaacagaaaccagggcaggctcctaaacttctcatacagttggccagcaatgttcaaaccggtgtgcccgcacgcttcagcggatcagggtcaggaaccgatttcacgctcacaattagcagtgtggagcctgatgacgtagccgtgtactactgtctgcaaagccgcaccatcccgagaacgtttgggggcggcacgaaattggaaattaaaggaagcacttccgggagtgggaagcctgggagtggcgaagggtcaacaaaggggcagatacagttggtccagagcgggcccgagttgaaaaagccaggtggatcagttaaaatttcttgtgctgcgtccggatatacttttactgactattctatcaactgggtcaaacaagctcccggtaaaggattggaatgggtcggatggataaacactgaaacccgcgagcctgcttatgcttacgattttcgaggtagattcaccttctcacttgatacatccaaaagtacggcttacctgcaaatgaactcactgagagcagaagataccgccgtttactattgtgccctcgactatagttacgccatggattattggggacaagggactctcgtgaccgtaagtagctttgtgcctgtgttcctgcctgccaagcctaccacaacaccagctcctagacctccaactcctgctcctacaatcgcctctcagccactgtctctgaggcctgaagcttgtagacctgctgctggcggagccgtgcataccagaggactggatttcgcctgcgacatctacatctgggctcctctggctggaacatgtggcgtgctgctgctgagcctggtcatcaccctgtactgcaaccaccggaacagagtgaagttcagcagatccgccgatgctcctgcctaccagcagggacagaaccagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcacagccaccaaggatacctatgatgccctgcacatgcaggccctgcctccaagatgagggcccgtttaaacccgctggctcgctttcttgctgtccaatttctattaaaggttcctttgttccgtaagtccaactactaaactgggggatattatgaagggccttgagcatctggattctgcctaataaaaaacatttattttcattgc

SEQ ID NO: 47 - DNA, 1644 bp, synthetic construct
  misc_feature 1..1644: note = Synthetic Polynucleotide
  source 1..1644: mol_type = other DNA
gggagacccaagctggctagcctcgcagttcggcggtcccgcgggtctgtctgttgcttcaacagtgtttggacggaacagatccggggactctcttccagccatggcactgcctgttacagctctgctgctgcctctggcactgctgcttcatgccgccagacctgatatacaactcacacaatcaccttcttctcttagcgcatcagtcggggatcgggttaccattacttgtcgcgcatcacagtccgtaacgatactcgggtcccatctgatccattggtatcagcaaaaaccaggcaaagcacccaagctcttgatccaattggcaagcaacgtgcaaactggagtcccttctcggttttccggaagtggctctggcactgactttacgttgaccattagtagcctccaacctgaggatgttgccacttattattgtcttcagtccagaaccattccgcgcacattcggacagggtactaagttggaaataaagggctcaacgtcaggctctggtaagccgggtagcggggagggatcaaccaaaggtcaagttcagctggtacaaagtggtggtggacttgtgcaacctggcaggtctgtgaaactctcctgtgccgctagcgggtacacttttactgactacagtataaactgggtgagacaagctccaggcaaaggactcgaatgggttgggtggataaatacagagactagagagccggcctacgcatacgacttcaggggaaggttcaccatcagtcgggacacttcaaagaatacggcgtaccttcaaatgaatagtttgagagcagaggacacagctgtgtattattgtgcgcgcgactactcctatgctatggactactgggggcaaggaaccttggtgactgtcagtagctttgtgcctgtgttcctgcctgccaagcctaccacaacaccagctcctagacctccaactcctgctcctacaatcgcctctcagccactgtctctgaggcctgaagcttgtagacctgctgctggcggagccgtgcataccagaggactggatttcgcctgcgacatctacatctgggctcctctggctggaacatgtggcgtgctgctgctgagcctggtcatcaccctgtactgcaaccaccggaacagagtgaagttcagcagatccgccgatgctcctgcctaccagcagggacagaaccagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcacagccaccaaggatacctatgatgccctgcacatgcaggccctgcctccaagatgagggcccgtttaaacccgctggctcgctttcttgctgtccaatttctattaaaggttcctttgttccgtaagtccaactactaaactgggggatattatgaagggccttgagcatctggattctgcctaataaaaaacatttattttcattgc

SEQ ID NO: 48 - protein, 246 aa, synthetic construct
  REGION 1..246: note = Synthetic Polypeptide
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 49 - protein, 246 aa, synthetic construct
  REGION 1..246: note = Synthetic Polypeptide
DIVMTQSPDSLSVSLGERATINCRASESVTILGSHLIHWYQQKPGQPPKLLIQLASNVQTGVPDRFSGSGSGTDFTLTISSLQAEDVAVYYCLQSRTIPRTFGGGTKVEIKGSTSGSGKPGSGEGSTKGQIQLVQSGAEVKKPGASVKISCKASGYTFTDYSINWVRQAPGQGLEWMGWINTETREPAYAYDFRGRVTMTRDTSASTAYLQISSLKAEDTAVYFCALDYSYAMDYWGQGSLVTVSS

SEQ ID NO: 50 - protein, 246 aa, synthetic construct
  REGION 1..246: note = Synthetic Polypeptide
DIVLTQSPASLAVSLGERATISCRASESVSILGSHLLHWYQQKPGQPPKLLIYLASNLQTGVPARFSGSGSGTDFTLTISSLEAEDVAVYYCLQSRTIPRTFGQGTKLEIKGSTSGSGKPGSGEGSTKGQIQLVQSGAEVKKPGASVKISCKASGYTFTDYSINWVRQAPGQGLEWMGWINTETREPAYAYDFRGRVTMTRDTSASTAYLQISSLKAEDTAVYFCALDYSYAMDYWGQGSLVTVSS

SEQ ID NO: 51 - protein, 246 aa, synthetic construct
  REGION 1..246: note = Synthetic Polypeptide
DIVLTQSPASLAVSLGKRATISCRASESVTILGSHLIHWYQQKPGQPPKLLIYLASNVQTGVPARFSGSGSGTDFTLTISSLEAEDVAVYYCLQSRTIPRTFGQGTKLEIKGSTSGSGKPGSGEGSTKGQVQLVQSGAEVKKPGASVKISCKASGYTFTDYSINWVRQAPGQGLEWMGWINTETREPAYAYDFRGRVTMTRDTSASTAYLQISSLKAEDTAVYFCALDYSYAMDYWGQGSLVTVSS

SEQ ID NO: 52 - protein, 246 aa, synthetic construct
  REGION 1..246: note = Synthetic Polypeptide
DIVMTQSPDSLSVSLGERATINCRASESVTILGSHLIHWYQQKPGQPPKLLIQLASNVQTGVPDRFSGSGSGTDFTLTISSLQAEDVAVYYCLQSRTIPRTFGGGTKVEIKGSTSGSGKPGSGEGSTKGQIQLVQSGAEVKKPGESVKISCKASGYTFTDYSINWVRQAPGQGLKWMGWINTETREPAYAYDFRGRFAFSLDTSASTAYLQISSLKAEDTAVYFCALDYSYAMDYWGQGTLVTVSS

SEQ ID NO: 53 - protein, 246 aa, synthetic construct
  REGION 1..246: note = Synthetic Polypeptide
DIVLTQSPASLAVSLGERATISCRASESVSILGSHYLAWYQQKPGQPPKLLIYLASNLQTGVPARFSGSGSGTDFTLTISSLEAEDVAVYYCQQSRTIPRTFGQGTKLEIKGSTSGSGKPGSGEGSTKGQIQLVQSGAEVKKPGESVKISCKASGYTFTDYSINWVRQAPGQGLKWMGWINTETREPAYAYDFRGRFAFSLDTSASTAYLQISSLKAEDTAVYFCALDYSYAMDYWGQGTLVTVSS

SEQ ID NO: 54 - protein, 246 aa, synthetic construct
  REGION 1..246: note = Synthetic Polypeptide
DIVLTQSPSSLSASVGDRATISCRASESVTILGSHLIHWYQQKPGQAPKLLIQLASNVQTGVPARFSGSGSGTDFTLTISSVEPDDVAVYYCLQSRTIPRTFGGGTKLEIKGSTSGSGKPGSGEGSTKGQIQLVQSGPELKKPGGSVKISCAASGYTFTDYSINWVKQAPGKGLEWVGWINTETREPAYAYDFRGRFTFSLDTSKSTAYLQMNSLRAEDTAVYYCALDYSYAMDYWGQGTLVTVSS

SEQ ID NO: 55 - protein, 246 aa, synthetic construct
  REGION 1..246: note = Synthetic Polypeptide
DIQLTQSPSSLSASVGDRVTITCRASQSVTILGSHLIHWYQQKPGKAPKLLIQLASNVQTGVPSRFSGSGSGTDFTLTISSLQPEDVATYYCLQSRTIPRTFGQGTKLEIKGSTSGSGKPGSGEGSTKGQVQLVQSGGGLVQPGRSVKLSCAASGYTFTDYSINWVRQAPGKGLEWVGWINTETREPAYAYDFRGRFTISRDTSKNTAYLQMNSLRAEDTAVYYCARDYSYAMDYWGQGTLVTVSS

SEQ ID NO: 56 - protein, 246 aa, synthetic construct
  REGION 1..246: note = Synthetic Polypeptide
DIVLTQSPSSLSASVGDRATISCRASESVTILGSHLIHWYQQKPGQAPKLLIQLASNVQTGVPARFSGSGSGTDFTLTISSVEPEDVAVYYCLQSRTIPRTFGQGTKVEIKGSTSGSGKPGSGEGSTKGQIQLVQSGPELKKPGGSVKISCAASGYTFTDYSINWVKQAPGKGLEWVGWINTETREPAYAYDFRGRFTFSADTSKSTAYLQMNSLRAEDTAVYYCALDYSYAMDYWGQGTLVTVSS

SEQ ID NO: 57 - protein, 246 aa, synthetic construct
  REGION 1..246: note = Synthetic Polypeptide
DIVLTQSPSSLSASVGDRATISCRASESVTILGSHLIHWYQQKPGQAPKLLIQLASNVQTGVPARFSGSGSGTDFTLTISSVEPEDVAVYYCLQSRTIPRTFGQGTKVEIKGSTSGSGKPGSGEGSTKGQIQLVQSGPELKKPGGSVKISCAASGYTFTDYSINWVRQAPGKGLEWVGWINTETREPAYAYDFRGRFTFSADTSKSTAYLQMNSLRAEDTAVYYCALDYSYAMDYWGQGTLVTVSS

SEQ ID NO: 58 - protein, 246 aa, synthetic construct
  REGION 1..246: note = Synthetic Polypeptide
DIVLTQSPSSLSASVGDRATISCRASESVTILGSHLIHWYQQKPGQAPKLLIQLASNVQTGVPARFSGSGSGTDFTLTISSVEPEDVAVYYCLQSRTIPRTFGQGTKVEIKGSTSGSGKPGSGEGSTKGQIQLVESGAEVKKPGGSVKISCAASGYTFTDYSINWVKQAPGKGLEWVGWINTETREPAYAYDFRGRFTFSLDTSKSTAYLQMNSLRAEDTAVYYCALDYSYAMDYWGQGTLVTVSS

SEQ ID NO: 59 - protein, 246 aa, synthetic construct
  REGION 1..246: note = Synthetic Polypeptide
DIVLTQSPSSLSASVGDRATISCRASESVTILGSHLIHWYQQKPGQAPKLLIQLASNVQTGVPARFSGSGSGTDFTLTISSVEPEDVAVYYCLQSRTIPRTFGQGTKVEIKGSTSGSGKPGSGEGSTKGQIQLVESGAEVKKPGGSVKVSCKASGYTFTDYSINWVRQAPGKGLEWVGWINTETREPAYAYDFRGRFAISAETSKSTAYLQMNSLRAEDTAVYYCALDYSYAMDYWGQGTLVTVSS

SEQ ID NO: 60 - protein, 246 aa, synthetic construct
  REGION 1..246: note = Synthetic Polypeptide
DIVLTQSPASLAVSPGQRATITCRASESVTILGSHLIHWYQQKPGQPPKLLIQLASNVQTGVPARFSGSGSGTDFTLTISSVEPEDTANYYCLQSRTIPRTFGQGTKVEIKGSTSGSGKPGSGEGSTKGQIQLVQSGPELKKPGGSVKISCKASGYTFTDYSINWVRQAPGKGLEWVGWINTETREPAYAYDFTGRFTFSADTSKSMAYLQINSLRAEDTAVYYCALDYSYAMDYWGQGTLVTVSS

SEQ ID NO: 61 - protein, 246 aa, synthetic construct
  REGION 1..246: note = Synthetic Polypeptide
DIVLTQSPASLAVSPGQRATISCRASESVTILGSHLIHWYQQKPGQPPKLLIQLASNVQTGVPARFSGSGSRTDFTLTISSLEPEDVAVYYCLQSRTIPRTFGQGTKVEIKGSTSGSGKPGSGEGSTKGQITLKESGPTLVKPTQTLTLSCKASGYTFTDYSINWVRRAPGKGLEWMGWINTETREPAYAYDFRGRFVFSLDTSVSMAYLQISSLKAEDTAVYYCALDYSYAMDYWGQGTLVTVSS

SEQ ID NO: 62 - protein, 246 aa, synthetic construct
  REGION 1..246: note = Synthetic Polypeptide
DIVLTQSPSSLSASVGDRATISCRASESVTILGSHLIHWYQQKPGQAPKLLIQLASNVQTGVPARFSGSGSGTDFTLTISSVEPEDVAVYYCLQSRTIPRTFGQGTKVEIKGSTSGSGKPGSGEGSTKGQIQLVQSGPELKKPGGSVKISCAASGYTFTSYDINWVRQAPGKGLEWVGWINTETREPAYAYDFRGRFTFSADTSKSTAYLQMNSLRAEDTAVYYCALDYSYAMDYWGQGTLVTVSS

SEQ ID NO: 63 - protein, 246 aa, synthetic construct
  REGION 1..246: note = Synthetic Polypeptide
DIVLTQSPSSLSASVGDRATISCRASESVTILGSHLIHWYQQKPGQAPKLLIQLASNVQTGVPARFSGSGSGTDFTLTISSVEPEDVAVYYCLQSRTIPRTFGQGTKVEIKGSTSGSGKPGSGEGSTKGQIQLVQSGPELKKPGGSVKISCAASGYTFTDYSINWVRQAPGKGLEWVGWINTETREPAYAYDFRGRFTFSADTSKSTAYLQMNSLRAEDTAVYYCALDYTYGMDYWGQGTLVTVSS

SEQ ID NO: 64 - protein, 246 aa, synthetic construct
  REGION 1..246: note = Synthetic Polypeptide
DIVLTQSPSSLSASVGDRATISCRASESVSFLGINLIHWYQQKPGQAPKLLIYSASNLQSGVPARFSGSGSGTDFTLTISSVEPEDVAVYYCLQSRTLPRTFGQGTKVEIKGSTSGSGKPGSGEGSTKGQIQLVQSGPELKKPGGSVKISCAASGYTFTDYSINWVRQAPGKGLEWVGWINTETREPAYAYDFRGRFTFSADTSKSTAYLQMNSLRAEDTAVYYCALDYSYAMDYWGQGTLVTVSS

SEQ ID NO: 65 - protein, 246 aa, synthetic construct
  REGION 1..246: note = Synthetic Polypeptide
DIVLTQSPSSLSASVGDRATISCRASESVSFLGINLIHWYQQKPGQAPKLLIYSASNLQSGVPARFSGSGSGTDFTLTISSVEPEDVAVYYCLQSKNFPRTFGQGTKVEIKGSTSGSGKPGSGEGSTKGQIQLVQSGPELKKPGGSVKISCAASGYTFTDYSINWVRQAPGKGLEWVGWINTETREPAYAYDFRGRFTFSADTSKSTAYLQMNSLRAEDTAVYYCALDYSYAMDYWGQGTLVTVSS

SEQ ID NO: 66 - protein, 246 aa, synthetic construct
  REGION 1..246: note = Synthetic Polypeptide
DIVLTQSPSSLSASVGDRATISCRASESVTILGSHLIHWYQQKPGQAPKLLIQLASNVQTGVPARFSGSGSGTDFTLTISSVEPEDVAVYYCLQSRTIPRTFGQGTKVEIKGSTSGSGKPGSGEGSTKGQIQLVQSGPELKKPGGSVKISCAASGYTFTSYSINWVRQAPGKGLEWVGWINTETREPAYAQGFTGRFTFSADTSKSTAYLQMNSLRAEDTAVYYCALDYLYSLDFWGQGTLVTVSS

SEQ ID NO: 67 - protein, 246 aa, synthetic construct
  REGION 1..246: note = Synthetic Polypeptide
DIVLTQSPSSLSASVGDRATISCRASESVTILGSHLIHWYQQKPGQAPKLLIQLASNVQTGVPARFSGSGSGTDFTLTISSVEPEDVAVYYCLQSRTIPRTFGQGTKVEIKGSTSGSGKPGSGEGSTKGQIQLVQSGPELKKPGGSVKISCAASGYTFTSYDINWVRQAPGKGLEWVGWINTNTGNPTYAQGFTGRFTFSADTSKSTAYLQMNSLRAEDTAVYYCALDYTYGMDYWGQGTLVTVSS

SEQ ID NO: 68 - protein, 246 aa, synthetic construct
  REGION 1..246: note = Synthetic Polypeptide
DIVLTQSPSSLSASVGDRATISCRASESVTILGSHLIHWYQQKPGQAPKLLIQLASNVQTGVPARFSGSGSGTDFTLTISSVEPEDVAVYYCLQSKNFPRTFGQGTKVEIKGSTSGSGKPGSGEGSTKGQIQLVQSGPELKKPGGSVKISCAASGYTFTSYDINWVRQAPGKGLEWVGWINTETREPAYAYDFRGRFTFSADTSKSTAYLQMNSLRAEDTAVYYCALDYLYSLDFWGQGTLVTVSS

SEQ ID NO: 69 - protein, 246 aa, synthetic construct
  REGION 1..246: note = Synthetic Polypeptide
DIVLTQSPSSLSASVGDRATISCRASESVSFLGINLIHWYQQKPGQAPKLLIYLASNLETGVPARFSGSGSGTDFTLTISSVEPEDVAVYYCLQSRTLPRTFGQGTKVEIKGSTSGSGKPGSGEGSTKGQIQLVQSGPELKKPGGSVKISCAASGYTFTSYSINWVRQAPGKGLEWVGWINTETREPAYAQGFTGRFTFSADTSKSTAYLQMNSLRAEDTAVYYCALDYLYSLDFWGQGTLVTVSS

SEQ ID NO: 70 - protein, 246 aa, synthetic construct
  REGION 1..246: note = Synthetic Polypeptide
DIVLTQSPSSLSASVGDRATISCRASESVTILGSHLIHWYQQKPGQAPKLLINLASNVNTGVPARFSGSGSGTDFTLTISSVEPEDVAVYYCLQSRTIPRTFGQGTKVEIKGSTSGSGKPGSGEGSTKGQIQLVQSGPELKKPGGSVKISCAASGYTFTDYSINWVRQAPGKGLEWVGWINTETREPAYAYDFRGRFTFSADTSKSTAYLQMNSLRAEDTAVYYCALDYSYAMDYWGQGTLVTVSS

SEQ ID NO: 71 - protein, 246 aa, synthetic construct
  REGION 1..246: note = Synthetic Polypeptide
DIVLTQSPSSLSASVGDRATISCRASESVTILGSHLIHWYQQKPGQAPKLLIQLASNVQTGVPARFSGSGSGTDFTLTISSVEPEDVAVYYCLQSRTLPRTFGQGTKVEIKGSTSGSGKPGSGEGSTKGQIQLVQSGPELKKPGGSVKISCAASGYTFTDYSINWVRQAPGKGLEWVGWINTETREPAYAYDFRGRFTFSADTSKSTAYLQMNSLRAEDTAVYYCALDYSYAMDYWGQGTLVTVSS

SEQ ID NO: 72 - protein, 246 aa, synthetic construct; identical to SEQ ID NO: 71
  REGION 1..246: note = Synthetic Polypeptide

SEQ ID NO: 73 - DNA, 103 bp, synthetic construct
  misc_feature 1..103: note = Synthetic Polynucleotide
  source 1..103: mol_type = other DNA
gggagacccaagctggctagcctcgcagttcggcggtcccgcgggtctgtctgttgcttcaacagtgtttggacggaacagatccggggactctcttccagcc

SEQ ID NO: 74 - DNA, 52 bp, synthetic construct
  misc_feature 1..52: note = Synthetic Polynucleotide
  source 1..52: mol_type = other DNA
agacccaagctggctagctctaaagaagcccctgggagcacagctcatcacc

SEQ ID NO: 75 - DNA, 89 bp, synthetic construct
  misc_feature 1..89: note = Synthetic Polynucleotide
  source 1..89: mol_type = other DNA
agacccaagctggctagcagctcctcacccaccccagccgcgactgtctccgccgagcccccggggccaggtgtcccgggcgcgccccg

SEQ ID NO: 76 - DNA, 73 bp, synthetic construct
  misc_feature 1..73: note = Synthetic Polynucleotide
  source 1..73: mol_type = other DNA
gtggaacacttctgaacctgcatttttatctggaactccagaagcagaatcctttgctaaataaatcgcagcc

SEQ ID NO: 77 - DNA, 137 bp, synthetic construct
  misc_feature 1..137: note = Synthetic Polynucleotide
  source 1..137: mol_type = other DNA
agagaagcagacatcttctagttcctcccccactctcctctttccggtacctgtgagtcagctaggggagggcagctctcacccaggctgatagttcggtgacctggctttatctactggatgagttccgctgggag

SEQ ID NO: 78 - DNA, 62 bp, synthetic construct
  misc_feature 1..62: note = Synthetic Polynucleotide
  source 1..62: mol_type = other DNA
agacccaagctggctagcatattgtgcttccaccactgccaataacaaaataactagcaacc

SEQ ID NO: 79 - DNA, 44 bp, synthetic construct
  misc_feature 1..44: note = Synthetic Polynucleotide
  source 1..44: mol_type = other DNA
aaataagagagaaaagaagagtaagaagaaatataagagccacc

SEQ ID NO: 80 - DNA, 73 bp, synthetic construct
  misc_feature 1..73: note = Synthetic Polynucleotide
  source 1..73: mol_type = other DNA
cgcgtccgccccgcgagcacagagcctcgcctttgccgatccgccgcccgtccacacccgccgccagctcacc

SEQ ID NO: 81 - DNA, 126 bp, synthetic construct
  misc_feature 1..126: note = Synthetic Polynucleotide
  source 1..126: mol_type = other DNA
cacattgttctgatcatctgaagatcagctattagaagagaaagatcagttaagtcctttggacctgatcagcttgatacaagaactactgatttcaacttctttggcttaattctctcggaaacg

SEQ ID NO: 82 - DNA, 104 bp, synthetic construct
  misc_feature 1..104: note = Synthetic Polynucleotide
  source 1..104: mol_type = other DNA
gtgtctgaaagcatttctggagtgttttaggcctgttcactttctcttactcactgtctattcacttgtcctgttcactcgtctggaagatctcagccagcacc

SEQ ID NO: 83 - DNA, 152 bp, synthetic construct
  misc_feature 1..152: note = Synthetic Polynucleotide
  source 1..152: mol_type = other DNA
gggcccgtttaaacccgctggctcgctttcttgctgtccaatttctattaaaggttcctttgttccgtaagtccaactactaaactgggggatattatgaagggccttgagcatctggattctgcctaataaaaaacatttattttcattgc

SEQ ID NO: 84 - DNA, 137 bp, synthetic construct
  misc_feature 1..137: note = Synthetic Polynucleotide
  source 1..137: mol_type = other DNA
tgagtgcgacggccggcaagcccccgctccccgggctctcgcggtcgcacgaggatgcttggcacgtaccccgtgtacatacttcccgggcgcccagcatggaaataaagcacccagcgctgccctgggcccctgcg

SEQ ID NO: 85 - DNA, 591 bp, synthetic construct
  misc_feature 1..591: note = Synthetic Polynucleotide
  source 1..591: mol_type = other DNA
gcggactatgacttagttgcgttacaccctttcttgacaaaacctaacttgcgcagaaaacaagatgagattggcatggctttatttgttttttttgttttgttttggtttttttttttttttggcttgactcaggatttaaaaactggaacggtgaaggtgacagcagtcggttggagcgagcatcccccaaagttctcaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacacctt

SEQ ID NO: 86 - DNA, 165 bp, synthetic construct
  misc_feature 1..165: note = Synthetic Polynucleotide
  source 1..165: mol_type = other DNA
attacttcaggggaagagatgacaaaacgagtctttcattcggtgtgaacttttctctttaattttaactgatttaacactttttgtgaattaatgaaatgataaagacttttatgtgagatttccttatcacagaaataaaatatctccaaatgtttccttttc

SEQ ID NO: 87 - DNA, 88 bp, synthetic construct
  misc_feature 1..88: note = Synthetic Polynucleotide
  source 1..88: mol_type = other DNA
acctgagactggtggcttctagaagcagccattaccaactgtaccttcccttcttgctcagccaataaatatatcctctttcactcag

SEQ ID NO: 88 - DNA, 583 bp, synthetic construct
  misc_feature 1..583: note = Synthetic Polynucleotide
  source 1..583: mol_type = other DNA
tggttgtcctgcctgcaatatttgaattttaaatctaaatctatttattaatatttaacattatttatatggggaatatatttttagactcatcaatcaaataagtatttataatagcaacttttgtgtaatgaaaatgaatatctattaatatatgtattatttataattcctatatcctgtgactgtctcacttaatcctttgttttctgactaattaggcaaggctatgtgattacaaggctttatctcaggggccaactaggcagccaacctaagcaagatcccatgggttgtgtgtttatttcacttgatgatacaatgaacacttataagtgaagtgatactatccagttactgccggtttgaaaatatgcctgcaatctgagccagtgctttaatggcatgtcagacagaacttgaatgtgtcaggtgaccctgatgaaaacatagcatctcaggagatttcatgcctggtgcttccaaatattgttgacaactgtgactgtacccaaatggaaagtaactcatttgttaaaattatcaatatctaatatatatgaataaagtgtaagttcacaac

SEQ ID NO: 89 - DNA, 123 bp, synthetic construct
  misc_feature 1..123: note = Synthetic Polynucleotide
  source 1..123: mol_type = other DNA
acacacctgcctgatgctatcaagaggctgaagaaagcgccaaatgtgctattttttggtcacttgctttatgacgtttattttcctgttaaagctgaataaataaaaactacagtaaatgta

SEQ ID NO: 90 - DNA, 897 bp, synthetic construct
  misc_feature 1..897: note = Synthetic Polynucleotide
  source 1..897: mol_type = other DNA
cctcaagatcaaggaaaggaggatggacgaacaggggccaaactggtgggaggcagaggtggtgggggcagggatgataggccctggatgtgcccacagggaccaagaagtgaggtttccactgtcttgcctgccagggcccctgttcccccgctggcagccaccccctcccccatcatatcctttgccccaaggctgctcagaggggccccggtcctggccccagcccccacctccgccccagacacaccccccagtcgagccctgcagccaaacagagccttcacaaccagccacacagagcctgcctcagctgctcgcacagattacttcagggctggaaaagtcacacagacacacaaaatgtcacaatcctgtccctcactcaacacaaaccccaaaacacagagagcctgcctcagtacactcaaacaacctcaaagctgcatcatcacacaatcacacacaagcacagccctgacaacccacacaccccaaggcacgcacccacagccagcctcagggcccacaggggcactgtcaacacaggggtgtgcccagaggcctacacagaagcagcgtcagtaccctcaggatctgaggtcccaacacgtgctcgctcacacacacggcctgttagaattcacctgtgtatctcacgcatatgcacacgcacagccccccagtgggtctcttgagtcccgtgcagacacacacagccacacacactgccttgccaaaaataccccgtgtctcccctgccactcacctcactcccattccctgagccctgatccatgcctcagcttagactgcagaggaactactcatttatttgggatccaaggcccccaacccacagtaccgtccccaataaactgcagccgagctccccac

SEQ ID NO: 91 - DNA, 97 bp, synthetic construct
  misc_feature 1..97: note = Synthetic Polynucleotide
  source 1..97: mol_type = other DNA
gaagtctgttcctgtcctccctgtgcagggtatcctgtagggtgacctggaattcgaattctgtttcccttgtaaaatatttgtctgtctctttttt

SEQ ID NO: 92 - DNA, 218 bp, synthetic construct
  misc_feature 1..218: note = Synthetic Polynucleotide
  source 1..218: mol_type = other DNA
ctatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagc

SEQ ID NO: 93 - DNA, 887 bp, synthetic construct
  misc_feature 1..887: note = Synthetic Polynucleotide
  source 1..887: mol_type = other DNA
agggatgctttaaccaaccctcttcctccccgtcattgtactgtaactgggacagaagaaataatggggatatgtggaatttttaacaacagttaaatggaaaagcatagacaattactgtagacatgataaaagaaacatttgtatgttcttagactcgaagtttgataaaagtaccttttcatgtggtgacagttgtgtgttgattggctaggtttctcccgtgtgttttatacaaaaatggaattgataaaccattttttacaaaattaatttgtctcaaaactgttctgttcatgatgtattagaaatattttactcagactttaaatattttaaatctcagattggttattcagagtaaccttagaacagaaattgggaatatatctttacaatgattgataccatggtatattgactcttagatgctattgatctgtagcaccattttttacaaacgactaaggaaaaaacctgccaattaaatcatgatatgccatcaattatgagacatcccaatttgagagatgttagattatagaaaagtatgcatttatgactgaaatggtagtggaattatttgaattctacaccaagcacttaccatgtgccaggccctttgcagagtgctctactgaccaagaaagttgttgctgccacattatagatgtggagcctaagggtcacagaaattgtgtgctatgccaaaaaacattgaactggtagatagaaaatgacagagctaggattcaaacctagatctggctgactccagagcctagttttacctggaattgatgttcagtttatcaaaggtttctccttttggtttaaaatcccaatttttggcctggcattgtggtttacgcctgtaatcccaacac

SEQ ID NO: 94 - DNA, 578 bp, synthetic construct
  misc_feature 1..578: note = Synthetic Polynucleotide
  source 1..578: mol_type = other DNA
cctggaagagggactccttgcctctccctatggcctgctggcccacctccctggaccccgttccaccctcacccttttcctttcccatgaaccctggagggtggtccccaccagctctttggaagtgagcagatgctgcggctggctttctgtcagcaggccggcctggcagtgggacaatcgccagagggtggggctggcagaacaccatctccagcctcagctttgacctgtctcatttcccatattccttcacacccagcttctggaaggcatggggtggctgggatttaaggacttctgggggaccaagacatcctcaagaaaacaggggcatccagggctccctggatgaatagaatgcaattcattcagaagctcagaagctaagaataagcctttgaaatacctcattgcatttccctttgggcttcggcttggggagatggatcaagctcagagactggcagtgagagcccagaaggacctgtataaaatgaatctggagctttacattttctgcctctgccttcctcccagctcagcaaggaagtatttgggcaccctaccctttacct

SEQ ID NO: 95 - DNA, 254 bp, synthetic construct
  misc_feature 1..254: note = Synthetic Polynucleotide
  source 1..254: mol_type = other DNA
accacgatcgttatgctgatcataccctaatgatcccagcaagataatgtcctgtcctctaagatgtgcatcaagcctggtacatactgaaaaccctataaggtcctggataatttttgtttgattattcattgaagaaacatttattttccaattgtgtgaagtttttgactgttaataaaagaatctgtcaaccatcaaagaggtctgcattatgcttgcatgtcaaaaactttaaaaatcctataatcttc

SEQ ID NO: 96 - DNA, 114 bp, synthetic construct
  misc_feature 1..114: note = Synthetic Polynucleotide
  source 1..114: mol_type = other DNA
gcggccgcttaattaagctgccttctgcggggcttgccttctggccatgcccttcttctctcccttgcacctgtacctcttggtctttgaataaagcctgagtaggaagtctag

SEQ ID NO: 97 - DNA, 1177 bp, synthetic construct
  misc_feature 1..1177: note = Synthetic Polynucleotide
  source 1..1177: mol_type = other DNA
gccctgctcgatgctcacagggcccccagcgagagtccctgcagtccctttcgacttgcatttttgcaggagcagtatcatgaagcctaaacgcgatggatatatgtttttgaaggcagaaagcaaaattatgtttgccactttgcaaaggagctcactgtggtgtctgtgttccaaccactgaatctggaccccatctgtgaataagccattctgactcatatcccctatttaacagggtctctagtgctgtgaaaaaaaaaatgctgaacattgcatataacttatattgtaagaaatactgtacaatgactttattgcatctgggtagctgtaaggcatgaaggatgccaagaagtttaaggaatatgggagaaatagtgtggaaattaagaagaaactaggtctgatattcaaatggacaaactgccagttttgtttcctttcactggccacagttgtttgatgcattaaaagaaaataaaaaaaagaaaaaagagaaaagaaaaaaaaagaaaaaagttgtaggcgaatcatttgttcaaagctgttggcctctgcaaaggaaataccagttctgggcaatcagtgttaccgttcaccagttgccgttgagggtttcagagagcctttttctaggcctacatgctttgtgaacaagtccctgtaattgttgtttgtatgtataattcaaagcaccaaaataagaaaagatgtagatttatttcatcatattatacagaccgaactgttgtataaatttatttactgctagtcttaagaactgctttctttcgtttgtttgtttcaatattttccttctctctcaatttttggttgaataaactagattacattcagttggcctaaggtggttgtgctcggagggtttcttgtttcttttccattttgtttttggatgatatttattaaatagcttctaagagtccggcggcatctgtcttgtccctattcctgcagcctgtgctgagggtagcagtgtatgagctaccagcgtgcatgtcagcgaccctggcccgacaggccacgtcctgcaatcggcccggctgcctcttcgccctgtcgtgttctgtgttagtgatcactgcctttaatacagtctgttggaataatattataagcataataataaagtgaaaatattttaaaactacaa

SEQ ID NO: 98 - DNA, 521 bp, synthetic construct
  misc_feature 1..521: note = Synthetic Polynucleotide
  source 1..521: mol_type = other DNA
gccctgctcgatgctcacagggcccccagcgagagtccctgcagtccctttcgacttgcatttttgcaggagcagtatcatgaagcctaaacgcgatggatatatgtttttgaaggcagaaagcaaaattatgtttgccactttgcaaaggagctcactgtggtgtctgtgttccaaccactgaatctggaccccatctgtgaataagccattctgactcatatcccctatttaacagggtctctagtgctgtgaaaaaaaaaatgctgaacattgcatataacttatattgtaagaaatactgtacaatgactttattgcatctgggtagctgtaaggcatgaaggatgccaagaagtttaaggaatatgggagaaatagtgtggaaattaagaagaaactaggtctgatattcaaatggacaaactgccagttttgtttcctttcactggccacagttgtttgatgcattaaaagaaaataaaaaaaagaaaaaagagaaaagaaaaaaaaagaaaaaa

SEQ ID NO: 99 - DNA, 656 bp, synthetic construct
  misc_feature 1..656: note = Synthetic Polynucleotide
  source 1..656: mol_type = other DNA
gttgtaggcgaatcatttgttcaaagctgttggcctctgcaaaggaaataccagttctgggcaatcagtgttaccgttcaccagttgccgttgagggtttcagagagcctttttctaggcctacatgctttgtgaacaagtccctgtaattgttgtttgtatgtataattcaaagcaccaaaataagaaaagatgtagatttatttcatcatattatacagaccgaactgttgtataaatttatttactgctagtcttaagaactgctttctttcgtttgtttgtttcaatattttccttctctctcaatttttggttgaataaactagattacattcagttggcctaaggtggttgtgctcggagggtttcttgtttcttttccattttgtttttggatgatatttattaaatagcttctaagagtccggcggcatctgtcttgtccctattcctgcagcctgtgctgagggtagcagtgtatgagctaccagcgtgcatgtcagcgaccctggcccgacaggccacgtcctgcaatcggcccggctgcctcttcgccctgtcgtgttctgtgttagtgatcactgcctttaatacagtctgttggaataatattataagcataataataaagtgaaaatattttaaaactacaa

SEQ ID NO: 100 - protein, 41 aa, synthetic construct
  REGION 1..41: note = Synthetic Polypeptide
RSKRSRLLHSDYMNMTPRRPGPTRKHYQPYAPPRDFAAYRS

SEQ ID NO: 101 - protein, 42 aa, synthetic construct
  REGION 1..42: note = Synthetic Polypeptide
KRGRKKLLYIFKQPFMRPVQTTQEEDGCSCRFPEEEEGGCEL

SEQ ID NO: 102 - protein, 37 aa, synthetic construct
  REGION 1..37: note = Synthetic Polypeptide
RRDQRLPPDAHKPPGGGSFRTPIQEEQADAHSTLAKI

SEQ ID NO: 103 - protein, 153 aa, synthetic construct
  REGION 1..153: note = Synthetic Polypeptide
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - protein, 154 aa, synthetic construct
  REGION 1..154: note = Synthetic Polypeptide
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 105 - protein, 149 aa, synthetic construct
  REGION 1..149: note = Synthetic Polypeptide
RRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 191 aa, synthetic construct
  REGION 1..191: note = Synthetic Polypeptide
KRGRKKLLYIFKQPFMRPVQTTQEEDGCSCRFPEEEEGGCELRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - protein, 112 aa, synthetic construct
  REGION 1..112: note = Synthetic Polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - protein, 18 aa, synthetic construct
  REGION 1..18: note = Synthetic Polypeptide
GSTSGSGKPGSGEGSTKG

SEQ ID NO: 109 - protein, 26 aa, synthetic construct
  REGION 1..26: note = Synthetic Polypeptide
GSTSGSGKPGSGEGSTKGSGGGSGGG

SEQ ID NO: 110 - protein, 21 aa, synthetic construct
  REGION 1..21: note = Synthetic Polypeptide
MALPVTALLLPLALLLHAARP

SEQ ID NO: 111 - protein, 83 aa, synthetic construct
  REGION 1..83: note = Synthetic Polypeptide
FVPVFLPAKPTTTPAPRPPTPAPTIASQPLSLRPEACRPAAGGAVHTRGLDFACDIYIWAPLAGTCGVLLLSLVITLYCNHRN

SEQ ID NO: 112 - DNA, 1515 bp, synthetic construct
  misc_feature 1..1515: note = Synthetic Polynucleotide
  source 1..1515: mol_type = other DNA
atggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgtccttcctgggcatcaacctgatccactggtaccagcagaagcccggacagccacccaaactgctgatctactccgccagcaacctgcagtcaggcgtgccagccagattcagcggcagcggcagcggaaccgacttcaccctgacaatcagctcagtggaaccagaagacaccgccaactactactgcctgcagagcagaaccctgcccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcacctcctacagcatcaactgggtgcgccaggccccaggcaagggcctggaatgggtcggctggatcaacaccgagacaagagagcccgcctacgcccagggcttcacaggcagattcaccttcagcgcagacacaagcaagagcatggcctacctgcagatcaactccctgagagcagaggacaccgccgtctactactgcgccctggactacctctactccctggacttctggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacaaacgcgggagaaaaaagctcctctacatcttcaagcaaccattcatgcgcccagtccaaaccacacaggaggaagacggatgcagctgcaggttccccgaagaggaggaaggcggatgcgagctcagagtgaagttcagcagatccgccgacgcaccagcctaccagcagggacagaaccagctgtacaacgagctgaacctggggagaagagaagagtacgacgtgctggataagcggagaggcagagaccctgagatgggcggcaagcccagacggaagaacccacaagagggcctgtacaacgagctgcagaaagacaagatggccgaggcctacagcgagatcggaatgaagggcgagcgcagaagaggcaagggacacgacggactgtaccagggcctgagcacagccaccaaggatacctacgatgccctgcacatgcaggccctgccaccaagatga

SEQ ID NO: 113 - DNA, 1515 bp, synthetic construct
  misc_feature 1..1515: note = Synthetic Polynucleotide
  source 1..1515: mol_type = other DNA
atggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgacaatcctgggctcccacctgatccactggtaccagcagaagcccggacagccacccaaactgctgatcaacctggccagcaacgtgaacacaggcgtgccagccagattcagcggcagcggcagcggaaccgacttcaccctgacaatcagctcagtggaaccagaagacaccgccaactactactgcctgcagagcagaacactgcccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcaccgactacagcatcaactgggtgcgccaggccccaggcaagggcctggaatgggtcggctggatcaacaccgagacaagagagcccgcctacgcctacgacttcacaggcagattcaccttcagcgcagacacaagcaagagcatggcctacctgcagatcaactccctgagagcagaggacaccgccgtctactactgcgccctggactacacctacggcatggactactggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacaaacgcgggagaaaaaagctcctctacatcttcaagcaaccattcatgcgcccagtccaaaccacacaggaggaagacggatgcagctgcaggttccccgaagaggaggaaggcggatgcgagctcagagtgaagttcagcagatccgccgacgcaccagcctaccagcagggacagaaccagctgtacaacgagctgaacctggggagaagagaagagtacgacgtgctggataagcggagaggcagagaccctgagatgggcggcaagcccagacggaagaacccacaagagggcctgtacaacgagctgcagaaagacaagatggccgaggcctacagcgagatcggaatgaagggcgagcgcagaagaggcaagggacacgacggactgtaccagggcctgagcacagccaccaaggatacctacgatgccctgcacatgcaggccctgccaccaagatga

SEQ ID NO: 114 - protein, 462 aa, synthetic construct
  REGION 1..462: note = Synthetic Polypeptide
MALPVTALLLPLALLLHAARPDIVLTQSPSSLSASVGDRATISCRASESVTILGSHLIHWYQQKPGQAPKLLIQLASNVQTGVPARFSGSGSGTDFTLTISSVEPEDVAVYYCLQSRTIPRTFGQGTKVEIKGSTSGSGKPGSGEGSTKGQIQLVESGAEVKKPGGSVKVSCKASGYTFTDYSINWVRQAPGKGLEWVGWINTETREPAYAYDFRGRFAISAETSKSTAYLQMNSLRAEDTAVYYCALDYSYAMDYWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 115 - DNA, 1389 bp, synthetic construct
  misc_feature 1..1389: note = Synthetic Polynucleotide
  source 1..1389: mol_type = other DNA
atggcactgcctgttaccgcgctgctcctcccccttgctctcctgcttcacgcagcgaggcctgacatcgtactcacccagtctccgagtagcctgtctgcaagtgtcggcgacagggcgacgataagttgcagagccagcgaaagcgtcacaattttgggttctcacttgattcactggtatcagcagaaacccggccaggcgccaaaacttttgattcagctcgcttcaaatgttcagaccggggtcccagctcggttctcaggctccggttcaggaacggacttcactcttaccatctcatccgtggagcctgaggatgttgctgtgtattattgtttgcagtccagaactatcccacgcacttttggccaaggcaccaaagtggagataaaaggatcaacatcaggaagcggcaagcctggttccggcgaaggctcaactaaaggtcagatccaattggtcgagtcaggtgcagaggttaaaaagcccggcggttctgtgaaggttagttgtaaggcatccgggtacacttttacggactatagcatcaattgggtgcgacaggcccccggaaaaggtcttgaatgggtgggttggattaataccgaaacgcgggaaccggcctatgcctatgactttagaggtcgcttcgctatttcagcagaaacctcaaagtccactgcgtatctgcagatgaacagtctccgggccgaggacaccgccgtttactactgcgccttggattatagctacgcaatggactattggggtcagggaaccctggtgactgttagcagcttcgttccagtgtttctgcccgctaagcccacaaccactcctgcaccgcgaccacccacacctgcgcctacaattgcgtcacagcccctgagccttcgaccggaagcctgtcggccagccgcaggcggggcggtccatactaggggactggactttgcttgtgatatttacatctgggctccccttgccggaacgtgtggcgttttgctcctttctctggtaataaccctgtattgcaatcaccggaatcgagtgaagttctccagaagcgccgatgcacccgcgtatcaacagggtcaaaatcaattgtataacgaacttaaccttgggaggcgcgaggagtacgatgtactcgacaaaagacgcggaagggaccccgaaatgggcggcaagccgcggaggaaaaacccgcaagagggactctataacgaacttcagaaggataagatggcagaagcctactcagagattggaatgaagggtgagcggcgcagggggaagggtcacgatggtctgtatcaaggactgtctacggcaaccaaagacacgtatgacgcattgcacatgcaagccttgcctccccggtga

SEQ ID NO: 116 - DNA, 1389 bp, synthetic construct
  misc_feature 1..1389: note = Synthetic Polynucleotide
  source 1..1389: mol_type = other DNA
atggctctgccggtaacagcgcttcttctcccgcttgccctcctcttgcacgctgctcggccagatattgtcctcacacagagcccgtcctcactttcagcgagcgtcggggaccgcgctaccatcagttgtcgagccagtgaaagcgtcactattctggggagccaccttatacattggtatcaacaaaagccaggccaggcccccaaactgctgattcagctcgcatctaatgttcagactggagtgcctgcaaggtttagcggtagtgggtcagggacagatttcaccttgaccattagctcagttgagcctgaagatgtggccgtttattactgcctccaaagccgcactatacctcggaccttcggccagggaacaaaggtggagattaagggtagtacctcaggtagtggcaagccagggagcggcgaggggtctacaaagggtcaaattcaattggtgcagtccggaccagaactgaaaaaaccgggcgggagcgtcaagatttcctgtgcagcatcaggctatacatttactgattacagtattaactgggtgagacaagcgcctggaaagggccttgagtgggtgggttggattaacacggagactagagaacccgcgtacgcctacgattttagaggccggttcactttctccgccgatacaagtaaatccacagcatatctccaaatgaatagccttagagcggaggataccgccgtgtattattgcgcgctcgactattcttacgcaatggactactgggggcagggtactctcgtcaccgtatcctcatttgttcccgtatttttgcccgccaagccgaccactactccggcccctcgacctccgacccctgctccgactattgcgagtcaaccattgagccttcggcctgaggcatgtagaccggcagctggtggcgcagtccatacacgagggcttgattttgcgtgcgacatctatatatgggcgccattggctgggacgtgcggcgtccttctcttgtcactcgtgataactctgtactgcaatcacagaaaccgcgtgaagttttccagaagcgcggacgcgccggcctatcaacaagggcagaatcagctgtacaacgaactcaatttggggcgaagggaggagtatgatgttttggacaaaaggcgaggccgcgaccctgaaatgggcggtaagccacgcagaaaaaacccccaagagggattgtacaatgaactgcaaaaagacaagatggctgaggcatacagtgagataggaatgaaaggtgaacggcgccggggaaaaggacatgacgggttgtaccagggtcttagcactgcaacgaaagatacctatgacgccctgcacatgcaagcattgcccccacggtga

SEQ ID NO: 117 - protein, 55 aa, synthetic construct
  REGION 1..55: note = Synthetic Polypeptide
FVPVFLPAKPTTTPAPRPPTPAPTIASQPLSLRPEACRPAAGGAVHTRGLDFACD

SEQ ID NO: 118 - protein, 21 aa, synthetic construct
  REGION 1..21: note = Synthetic Polypeptide
IYIWAPLAGTCGVLLLSLVIT

SEQ ID NO: 119 - protein, 7 aa, synthetic construct
  REGION 1..7: note = Synthetic Polypeptide
LYCNHRN

SEQ ID NO: 120 - protein, 28 aa, synthetic construct
  REGION 1..28: note = Synthetic Polypeptide
IYIWAPLAGTCGVLLLSLVITLYCNHRN

SEQ ID NO: 121 - protein, 184 aa, synthetic construct
  REGION 1..184: note = Synthetic Polypeptide
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 122 - DNA, 1389 bp, synthetic construct
  misc_feature 1..1389: note = Synthetic Polynucleotide
  source 1..1389: mol_type = other DNA
atggcactgcctgttacagctctgctgctgcctctggcactgctgcttcatgccgccagacctgacatcgtgctgacacagtctccttccagcctgagcgcatctgtcggcgacagagccacaatcagctgcagagccagcgagtctgtgacaatcctgggctctcacctgatccactggtatcagcagaagcccggacaggcacctaaactgctgatccagctggcctctaatgtgcagacaggcgtgccagccagattttctggcagcggcagcggaaccgacttcaccctgacaatcagctcagtggaaccagaagatgtggccgtgtactactgcctgcagagcagaacaatccccagaacctttggccaaggcaccaaggtcgaaatcaagggctctacaagcggcagcggaaagcctggatctggcgagggatctaccaagggccagattcagctggtgcagtctggccccgaactgaagaaacctggcggaagcgtgaaaatctcctgcgcagccagcggctacaccttcaccgactacagcatcaactgggttcgccaggcccctggcaagggcctggaatgggtcggctggatcaacaccgagactagagagcccgcctacgcctacgacttcagaggcagattcaccttcagcgcagacacaagcaagagcacagcctacctgcagatgaactccctgagagcagaggacaccgccgtctactattgcgccctggattacagctacgccatggattattggggccagggcaccctcgtgaccgtgtcatcttttgtgcctgtgttcctgcctgccaagcctaccacaacaccagctcctagacctccaactcctgctcctacaatcgcctctcagccactgtctctgaggcctgaggcatgtagacctgctgctggcggagccgtgcataccagaggactggatttcgcctgcgacatctacatctgggctcctctggctggaacatgtggcgtgctgctgctgagcctggtcatcaccctgtactgcaaccaccggaacagagtgaagttcagcagatccgccgatgctcctgcctaccagcagggacagaaccagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcacagccaccaaggatacctatgatgccctgcacatgcaggccctgcctccaagatga

SEQ ID NO: 123 - DNA, 1389 bp, synthetic construct
  misc_feature 1..1389: note = Synthetic Polynucleotide
  source 1..1389: mol_type = other DNA
atggcactgccggtaacagcgctcctcctcccgctcgccctcctcctccacgcagcacggccagacatcgtcctcacacagagcccgagcagcctcagcgcgagcgtcggggaccgcgcaaccatcagctgccgagccagcgaaagcgtcacaatcctcgggagccacctgatacactggtaccaacaaaagccaggccaggcccccaaactgctgatccagctcgcaagcaacgtccagacaggagtgccagcaaggttcagcggaagcgggagcggaacagacttcaccctcaccatcagcagcgtcgagccagaagatgtggccgtctactactgcctccaaagccgcacaataccacggaccttcggccagggaacaaaggtggagatcaagggaagcaccagcggaagcggcaagccagggagcggcgaggggagcacaaagggacaaatccaactcgtgcagagcggaccagaactgaaaaagccgggcggaagcgtcaagatcagctgcgcagcaagcggctacacattcacagactacagcatcaactgggtgagacaagcgccaggaaagggcctcgaatgggtgggatggatcaacacggagacaagagaacccgcatacgcctacgacttcagaggccggttcacattcagcgccgacacaagcaagagcacagcatacctccaaatgaacagcctcagagcggaggacaccgccgtgtactactgcgcgctcgactacagctacgcaatggactactggggacagggaacactcgtcaccgtaagcagcttcgtacccgtattcctccccgccaagccgaccacaacaccggccccacgaccaccaacaccagccccgacaatcgcgagccaaccactcagcctccggccagaggcgtgcagaccggcagcaggaggcgcagtccacacacgaggactcgacttcgcgtgcgacatatacatctgggcaccactcgcaggaacgtgcggcgtcctcctcctcagcctcgtgataacactgtactgcaaccacagaaaccgcgtgaagttcagcagaagcgcggacgcgccggcctaccaacaaggacagaaccagctgtacaacgaactcaacctcggacgaagggaggagtacgacgtcctcgacaaaaggcgaggccgcgacccagaaatgggcggaaagccacgcagaaaaaacccccaagagggactctacaacgaactgcaaaaggacaagatggcagaggcatacagcgagataggaatgaaaggagaacggcgccggggaaaaggacacgacgggctctaccagggactcagcacagcaacgaaagacacctacgacgccctgcacatgcaagcactccccccacggtga

SEQ ID NO: 124 - protein, 5 aa, synthetic construct
  REGION 1..5: note = Synthetic Polypeptide
GGGGS

SEQ ID NO: 125 - protein, 10 aa, synthetic construct
  REGION 1..10: note = Synthetic Polypeptide
GGGGSGGGGS

SEQ ID NO: 126 - protein, 15 aa, synthetic construct
  REGION 1..15: note = Synthetic Polypeptide
GGGGSGGGGSGGGGS

SEQ ID NO: 127 - protein, 18 aa, synthetic construct
  REGION 1..18: note = Synthetic Polypeptide
GSTSGGGSGGGSGGGGSS

SEQ ID NO: 128 - protein, 18 aa, synthetic construct
  REGION 1..18: note = Synthetic Polypeptide
GSTSGSGKPGSSEGSTKG

SEQ ID NO: 129 - protein, 8 aa, synthetic construct
  REGION 1..8: note = Synthetic Polypeptide
GGGGSGGG

SEQ ID NO: 130 - protein, 4 aa, synthetic construct
  REGION 1..4: note = Synthetic Polypeptide
GGGS

SEQ ID NO: 131 - protein, 15 aa, synthetic construct
  REGION 1..15: note = Synthetic Polypeptide
GLYGLYGLYGLYSER

SEQ ID NO: 132 - protein, 60 aa, synthetic construct
  REGION 1..60: note = Synthetic Polypeptide
GLYGLYGLYGLYSERGLYGLYGLYGLYSERGLYGLYGLYGLYSERGLYGLYGLYGLYSER

SEQ ID NO: 133 - protein, 45 aa, synthetic construct
  REGION 1..45: note = Synthetic Polypeptide
GLYGLYGLYGLYSERGLYGLYGLYGLYSERGLYGLYGLYGLYSER